SEQUENCE LISTING (174 sequences)

SEQ ID NO: 1 - protein, 133 aa, Artificial Sequence
  synthetic sequence _clone 2_6B8 alpha chain variable domain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVPDDAGNMLTFGGGTRLMVKPH

SEQ ID NO: 2 - protein, 134 aa, Artificial Sequence
  synthetic sequence clone3_6C3 alpha chain variable domain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCSSGGGADGLTFGKGTHLIIQPY

SEQ ID NO: 3 - protein, 134 aa, Artificial Sequence
  synthetic sequence clone6_5G8 alpha chain variable domain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCASGGGADGLTFGKGTHLIIQPY

SEQ ID NO: 4 - protein, 137 aa, Artificial Sequence
  synthetic sequence clone15_3F10 alpha chain variable domain
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCASMYSGGGADGLTFGKGTHLIIQPY

SEQ ID NO: 5 - protein, 134 aa, Artificial Sequence
  synthetic sequence clone17_9B5 alpha chain variable domain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAGGGGADGLTFGKGTHLIIQPY

SEQ ID NO: 6 - protein, 139 aa, Artificial Sequence
  synthetic sequence clone19_5C2 alpha chain variable domain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSGSDGGSQGNLIFGKGTKLSVKPN

SEQ ID NO: 7 - protein, 128 aa, Artificial Sequence
  synthetic sequence clone21_7A10 alpha chain variable domain
MWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAVRDDYGQNFVFGPGTRLSVLPY

SEQ ID NO: 8 - protein, 138 aa, Artificial Sequence
  synthetic sequence clone 2_6B8 beta chain variable domain
MGTRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYFCASSELPAGGTNEQFFGPGTRLTVLDLKN

SEQ ID NO: 9 - protein, 135 aa, Artificial Sequence
  synthetic sequence clone3_6C3 beta chain variable domain
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASMDLAFKQYFGPGTRLTVTEDLKN

SEQ ID NO: 10 - protein, 135 aa, Artificial Sequence
  synthetic sequence clone6_5G8 beta chain variable domain
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSFLTDTQYFGPGTRLTVLEDLKN

SEQ ID NO: 11 - protein, 137 aa, Artificial Sequence
  synthetic sequence clone15_3F10 beta chain variable domain
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSFFSNTGELFFGEGSRLTVLEDLNK

SEQ ID NO: 12 - protein, 135 aa, Artificial Sequence
  synthetic sequence clone17_9B5 beta chain variable domain
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYMGPEAFFGQGTRLTVVEDLNK

SEQ ID NO: 13 - protein, 139 aa, Artificial Sequence
  synthetic sequence clone19_5C2 beta chain variable domain
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQVSGYEQYFGPGTRLTVTEDLKN

SEQ ID NO: 14 - protein, 137 aa, Artificial Sequence
  synthetic sequence clone21_7A10 beta chain variable domain
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPQGDNEQFFGPGTRLTVLEDLNK

SEQ ID NO: 15 - protein, 141 aa, Artificial Sequence
  synthetic sequence alpha chain constant domain
DIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 16 - protein, 176 aa, Artificial Sequence
  synthetic sequence beta chain constant domain Cbeta1Cys
DLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 17 - protein, 178 aa, Artificial Sequence
  synthetic sequence beta chain constant domain Cbeta2Cys
DLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 18 - DNA, 397 bp, Artificial Sequence
  synthetic sequence clone 2_6B8 alpha chain variable domain
atgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtcccggacgatgcaggcaacatgctcacctttggagggggaacaaggttaatggtcaaacccc

SEQ ID NO: 19 - DNA, 400 bp, Artificial Sequence
  synthetic sequence clone3_6C3 alpha chain variable domain
atggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgttcatcaggaggaggtgctgacggactcacctttggcaaagggactcatctaatcatccagccct

SEQ ID NO: 20 - DNA, 400 bp, Artificial Sequence
  synthetic sequence clone6_5G8 alpha chain variable domain
atggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcctctggaggaggtgctgacggactcacctttggcaaagggactcatctaatcatccagccct

SEQ ID NO: 21 - DNA, 409 bp, Artificial Sequence
  synthetic sequence clone15_3F10 alpha chain variable domain
atgacacgagttagcttgctgtgggcagtcgtggtctccacctgtcttgaatccggcatggcccagacagtcactcagtctcaaccagagatgtctgtgcaggaggcagagactgtgaccctgagttgcacatatgacaccagtgagaataattattatttgttctggtacaagcagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgcctcgatgtattcaggaggaggtgctgacggactcacctttggcaaagggactcatctaatcatccagccct

SEQ ID NO: 22 - DNA, 400 bp, Artificial Sequence
  synthetic sequence clone17_9B5 alpha chain variable domain
atggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgctggggggggaggtgctgacggactcacctttggcaaagggactcatctaatcatccagccct

SEQ ID NO: 23 - DNA, 415 bp, Artificial Sequence
  synthetic sequence clone19_5C2 alpha chain variable domain
atggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttataggagcggaagtgatggaggaagccaaggaaatctcatctttggaaaaggcactaaactctctgttaaaccaa

SEQ ID NO: 24 - DNA, 382 bp, Artificial Sequence
  synthetic sequence clone21_7A10 alpha chain variable domain
atgtggggagctttccttctctatgtttccatgaagatgggaggcactgcaggacaaagccttgagcagccctctgaagtgacagctgtggaaggagccattgtccagataaactgcacgtaccagacatctgggttttatgggctgtcctggtaccagcaacatgatggcggagcacccacatttctttcttacaatgctctggatggtttggaggagacaggtcgtttttcttcattccttagtcgctctgatagttatggttacctccttctacaggagctccagatgaaagactctgcctcttacttctgcgctgtgagagatgactatggtcagaattttgtctttggtcccggaaccagattgtccgtgctgccct

SEQ ID NO: 25 - DNA, 414 bp, Artificial Sequence
  synthetic sequence 2_6B8 beta chain variable domain
atgggcaccaggctcttcttctatgtggccctttgtctgctgtgggcaggacacagggatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtccccgatggctatgttgtctccagatccaagacagagaatttccccctcactctggagtcagctacccgctcccagacatctgtgtatttctgcgccagcagtgagctcccagcgggagggacgaatgagcagttcttcgggccagggacacggctcaccgtgctagacctgaaaaac

SEQ ID NO: 26 - DNA, 405 bp, Artificial Sequence
  synthetic sequence clone3_6C3 beta chain variable domain
atgagcctcgggctcctgtgctgtggggccttttctctcctgtgggcaggtccagtgaatgctggtgtcactcagaccccaaaattccgggtcctgaagacaggacagagcatgacactgctgtgtgcccaggatatgaaccatgaatacatgtactggtatcgacaagacccaggcatggggctgaggctgattcattactcagttggtgagggtacaactgccaaaggagaggtccctgatggctacaatgtctccagattaaaaaaacagaatttcctgctggggttggagtcggctgctccctcccaaacatctgtgtacttctgtgccagcatggatctagcgtttaagcagtacttcgggccgggcaccaggctcacggtcacagaggacctgaaaaac

SEQ ID NO: 27 - DNA, 405 bp, Artificial Sequence
  synthetic sequence clone6_5G8 beta chain variable domain
atgagcctcgggctcctgtgctgtggggccttttctctcctgtgggcaggtccagtgaatgctggtgtcactcagaccccaaaattccgggtcctgaagacaggacagagcatgacactgctgtgtgcccaggatatgaaccatgaatacatgtactggtatcgacaagacccaggcatggggctgaggctgattcattactcagttggtgagggtacaactgccaaaggagaggtccctgatggctacaatgtctccagattaaaaaaacagaatttcctgctggggttggagtcggctgctccctcccaaacatctgtgtacttctgtgccagcagtttccttacagatacgcagtattttgggccgggcaccaggctcacggtcacagaggacctgaaaaac

SEQ ID NO: 28 - DNA, 411 bp, Artificial Sequence
  synthetic sequence clone15_3F10 beta chain variable domain
atgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttcttctcgaacaccggggagctgttttttggagaaggctctaggctgaccgtactggaggacctgaacaag

SEQ ID NO: 29 - DNA, 405 bp, Artificial Sequence
  synthetic sequence clone17_9B5 beta chain variable domain
atgagcctcgggctcctgtgctgtggggccttttctctcctgtgggcaggtccagtgaatgctggtgtcactcagaccccaaaattccgggtcctgaagacaggacagagcatgacactgctgtgtgcccaggatatgaaccatgaatacatgtactggtatcgacaagacccaggcatggggctgaggctgattcattactcagttggtgagggtacaactgccaaaggagaggtccctgatggctacaatgtctccagattaaaaaaacagaatttcctgctggggttggagtcggctgctccctcccaaacatctgtgtacttctgtgccagcagttacatggggcctgaagctttctttggacaaggcaccagactcacagttgtagaggacctgaacaag

SEQ ID NO: 30 - DNA, 417 bp, Artificial Sequence
  synthetic sequence clone19_5C2 beta chain variable domain
atgggctgcaggctgctctgctgtgcggttctctgtctcctgggagcgggtgagttggtccccatggaaacgggagttacgcagacaccaagacacctggtcatgggaatgacaaataagaagtctttgaaatgtgaacaacatctgggtcataacgctatgtattggtacaagcaaagtgctaagaagccactggagctcatgtttgtctacagtcttgaagaacgggttgaaaacaacagtgtgccaagtcgcttctcacctgaatgccccaacagctctcacttattccttcacctacacaccctgcagccagaagactcggccctgtatctctgcgccagcagccaagtttcgggttacgagcagtacttcgggccgggcaccaggctcacggtcacagaggacctgaaaaac

SEQ ID NO: 31 - DNA, 411 bp, Artificial Sequence
  synthetic sequence clone21_7A10 beta chain variable domain
atggacaccagagtactctgctgtgcggtcatctgtcttctgggggcaggtctctcaaatgccggcgtcatgcagaacccaagacacctggtcaggaggaggggacaggaggcaagactgagatgcagcccaatgaaaggacacagtcatgtttactggtatcggcagctcccagaggaaggtctgaaattcatggtttatctccagaaagaaaatatcatagatgagtcaggaatgccaaaggaacgattttctgctgaatttcccaaagagggccccagcatcctgaggatccagcaggtagtgcgaggagattcggcagcttatttctgtgccagctcaccacagggtgacaatgagcagttcttcgggccagggacacggctcaccgtgctagaggacctgaacaag

SEQ ID NO: 32 - DNA, 397 bp, Artificial Sequence
  synthetic sequence 2_6B8 alpha chain variable domain
atgaagtccctgcgggtgctgctcgtgatcctgtggctccagctgagctgggtgtggtcccagcagaaagaggtggaacagaacagcggccctctgagcgtgccagaaggcgctatcgccagcctgaactgcacctacagcgacagaggcagccagagcttcttctggtacagacagtacagcggcaagagccccgagctgatcatgttcatctacagcaacggcgacaaagaggacggccggttcaccgcccagctgaacaaggccagccagtacgtgtccctgctgatcagagacagccagcccagcgacagcgccacctatctgtgtgccgtgcctgacgacgccggcaacatgctgacatttggcggcggaacccggctgatggtcaagcccc

SEQ ID NO: 33 - DNA, 400 bp, Artificial Sequence
  synthetic sequence clone3_6C3 alpha chain variable domain
atggcctgccccggatttctgtgggccctcgtgatcagcacctgtctggaattcagcatggcccagaccgtgacccagagccagcctgagatgagcgtgcaggaagccgagacagtgaccctgagctgcacctacgacaccagcgagagcgactactacctgttctggtacaagcagccccccagccggcagatgatcctcgtgattagacaggaagcctataagcagcagaacgccaccgagaacagattcagcgtgaacttccagaaggccgccaagagcttcagcctgaagatcagcgacagccagctgggcgacgccgccatgtacttttgttctagcggcggaggcgccgacggcctgacatttggaaagggcacccacctgatcatccagccct

SEQ ID NO: 34 - DNA, 400 bp, Artificial Sequence
  synthetic sequence clone6_5G8 alpha chain variable domain
atggcctgccccggatttctgtgggccctcgtgatcagcacctgtctggaattcagcatggcccagaccgtgacccagagccagcctgagatgagcgtgcaggaagccgagacagtgaccctgagctgcacctacgacaccagcgagagcgactactacctgttctggtacaagcagccccccagccggcagatgatcctcgtgattagacaggaagcctataagcagcagaacgccaccgagaacagattcagcgtgaacttccagaaggccgccaagagcttcagcctgaagatcagcgacagccagctgggcgacgccgccatgtacttttgtgctagcggaggcggagccgacggcctgacatttggcaagggcacccacctgatcatccagccct

SEQ ID NO: 35 - DNA, 409 bp, Artificial Sequence; identical to SEQ ID NO: 21
  synthetic sequence clone15_3F10 alpha chain variable domain

SEQ ID NO: 36 - DNA, 400 bp, Artificial Sequence
  synthetic sequence clone17_9B5 alpha chain variable domain
atggcctgccccggatttctgtgggccctcgtgatcagcacctgtctggaattcagcatggcccagaccgtgacccagagccagcctgagatgagcgtgcaggaagccgagacagtgaccctgagctgcacctacgacaccagcgagagcgactactacctgttctggtacaagcagccccccagccggcagatgatcctcgtgattagacaggaagcctataagcagcagaacgccaccgagaacagattcagcgtgaacttccagaaggccgccaagagcttcagcctgaagatcagcgacagccagctgggcgacgccgccatgtatttttgtgctggcggaggcggagccgacggcctgacatttggaaagggcacccacctgatcatccagccct

SEQ ID NO: 37 - DNA, 415 bp, Artificial Sequence
  synthetic sequence clone19_5C2 alpha chain variable domain
atggcctgccccggatttctgtgggccctcgtgatcagcacctgtctggaattcagcatggcccagaccgtgacccagagccagcctgagatgagcgtgcaggaagccgagacagtgaccctgagctgcacctacgacaccagcgagagcgactactacctgttctggtacaagcagccccccagccggcagatgatcctcgtgattagacaggaagcctataagcagcagaacgccaccgagaacagattcagcgtgaacttccagaaggccgccaagagcttcagcctgaagatcagcgacagccagctgggcgacgccgccatgtacttttgcgcctatagaagcggcagcgacggcggctctcagggcaatctgatcttcggcaagggcaccaagctgagcgtgaagccca

SEQ ID NO: 38 - DNA, 382 bp, Artificial Sequence
  synthetic sequence clone21_7A10 alpha chain variable domain
atgtggggcgcctttctgctgtacgtgtccatgaagatgggcggcacagccggccagagcctggaacagccttctgaagtgaccgccgtggaaggcgccatcgtgcagatcaactgcacctaccagaccagcggcttctacggcctgagctggtatcagcagcatgacggcggagcccccaccttcctgagctacaatgccctggacggcctggaagagacaggccggttcagcagcttcctgtccagaagcgacagctacggctacctgctgctccaggaactccagatgaaggacagcgccagctacttctgcgccgtgcgggatgactacggccagaacttcgtgttcggccctggcaccagactgagcgtgctgccct

SEQ ID NO: 39 - DNA, 414 bp, Artificial Sequence
  synthetic sequence 2_6B8 beta chain variable domain
atgggcaccagactgtttttctacgtggccctgtgcctgctgtgggccggacatagagatgccggaatcacccagagcccccggtacaagatcaccgagacaggcagacaagtgaccctgatgtgccaccagacctggtcccacagctacatgttctggtacagacaggacctgggccacggcctgcggctgatctactattctgccgccgctgacatcaccgacaagggcgaagtgcccgacggctacgtggtgtccagaagcaagaccgagaacttcccactgaccctggaaagcgccacccggtcccagaccagcgtgtacttttgtgccagcagcgagctgcccgctggcggcaccaatgagcagttttttggccctggcacccggctgaccgtgctggacctgaagaat

SEQ ID NO: 40 - DNA, 405 bp, Artificial Sequence
  synthetic sequence clone3_6C3 beta chain variable domain
atgtctctgggactgctgtgttgcggcgccttcagcctgctgtgggccggacctgtgaatgccggcgtgacccagacccccaagttccgggtgctgaaaaccggccagagcatgaccctgctgtgcgcccaggacatgaaccacgagtacatgtattggtacagacaggaccccggcatgggcctgcggctgatccactattctgtgggcgagggcaccaccgccaagggcgaagtgcctgatggctacaacgtgtcccggctgaagaagcagaacttcctgctgggcctggaaagcgccgctcctagccagaccagcgtgtacttctgcgccagcatggacctggccttcaagcagtacttcggccctggcaccagactgaccgtgaccgaggacctgaagaac

SEQ ID NO: 41 - DNA, 405 bp, Artificial Sequence
  synthetic sequence clone6_5G8 beta chain variable domain
atgtctctgggactgctgtgttgcggcgccttcagcctgctgtgggccggacctgtgaatgccggcgtgacccagacccccaagttccgggtgctgaaaaccggccagagcatgaccctgctgtgcgcccaggacatgaaccacgagtacatgtattggtacagacaggaccccggcatgggcctgcggctgatccactattctgtgggcgagggcaccaccgccaagggcgaagtgcctgatggctacaacgtgtcccggctgaagaagcagaacttcctgctgggcctggaaagcgccgctcctagccagaccagcgtgtacttctgcgccagcagcttcctgaccgacacccagtactttggccctggcaccagactgaccgtgctggaagatctgaagaac

SEQ ID NO: 42 - DNA, 411 bp, Artificial Sequence
  synthetic sequence clone15_3F10 beta chain variable domain
atgggacctcagctgctgggatacgtggtgctgtgtctgctgggagccggacctctggaagcccaagtgacccagaaccccagatacctgatcaccgtgaccggcaagaaactgaccgtgacctgctcccagaacatgaaccacgagtacatgagctggtacagacaggaccccggcctgggcctgcggcagatctactacagcatgaacgtggaagtgaccgacaagggcgacgtgcccgagggctacaaggtgtcccggaaagagaagcggaacttcccactgatcctggaaagccccagccccaaccagaccagcctgtacttctgcgccagcagcttcttcagcaacaccggcgagctgttcttcggcgagggcagcagactgacagtgctggaagatctgaacaag

SEQ ID NO: 43 - DNA, 405 bp, Artificial Sequence
  synthetic sequence clone17_9B5 beta chain variable domain
atgtctctgggactgctgtgttgcggcgccttcagcctgctgtgggccggacctgtgaatgccggcgtgacccagacccccaagttccgggtgctgaaaaccggccagagcatgaccctgctgtgcgcccaggacatgaaccacgagtacatgtattggtacagacaggaccccggcatgggcctgcggctgatccactattctgtgggcgagggcaccaccgccaagggcgaagtgcctgatggctacaacgtgtcccggctgaagaagcagaacttcctgctgggcctggaaagcgccgctcctagccagaccagcgtgtacttctgcgccagcagctatatgggccccgaggcattctttgggcagggaaccagactgaccgtggtggaagatctgaacaag

SEQ ID NO: 44 - DNA, 417 bp, Artificial Sequence
  synthetic sequence clone19_5C2 beta chain variable domain
atgggatgtagactgctgtgttgcgccgtgctgtgcctgctgggagctggcgaactggtgcctatggaaaccggcgtgacccagacccccagacacctcgtgatgggcatgaccaacaagaaaagcctgaagtgcgagcagcacctgggccacaacgccatgtactggtacaagcagagcgccaagaaacccctggaactgatgttcgtgtacagcctggaagagagggtggaaaacaacagcgtgcccagccggttcagccccgagtgccctaatagcagccacctgtttctgcatctgcacaccctccagcccgaggacagcgccctgtatctgtgtgccagctctcaggtgtccggctacgagcagtacttcggccctggcaccagactgaccgtgaccgaggacctgaagaac

SEQ ID NO: 45 - DNA, 411 bp, Artificial Sequence
  synthetic sequence clone21_7A10 beta chain variable domain
atggatacaagagtgctgtgctgcgccgtgatctgcctgctgggagccggactgtctaatgccggcgtgatgcagaaccccagacacctcgtgcggcggagaggacaggaagccagactgcgctgtagccccatgaagggccacagccacgtgtactggtacagacagctgcccgaagagggcctgaagttcatggtgtacctccagaaagagaacatcatcgacgagagcggcatgcccaaagagcggttcagcgccgagttccccaaagagggccccagcatcctgagaatccagcaggtcgtgcggggcgatagcgccgcctatttttgtgccagcagcccccagggcgacaacgagcagttttttggccctggcacccggctgaccgtgctggaagatctgaacaag

SEQ ID NO: 46 - DNA, 426 bp, Artificial Sequence
  synthetic sequence Universal codon optimized alpha chain constant domain
gatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 47 - DNA, 531 bp, Artificial Sequence
  synthetic sequence Universal codon optimized beta chain constant domain Cbeta1Cys
gacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 48 - DNA, 537 bp, Artificial Sequence
  synthetic sequence 2_6B8 beta chain constant domain Cbeta2Cys
gacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 49 - protein, 13 aa, Artificial Sequence
  synthetic sequence clone2_6B8 alpha chain CDR3
CAVPDDAGNMLTF

SEQ ID NO: 50 - protein, 12 aa, Artificial Sequence
  synthetic sequence clone3_6C3 alpha chain CDR3
CSSGGGADGLTF

SEQ ID NO: 51 - protein, 12 aa, Artificial Sequence
  Synthetic sequence clone6_5G8 alpha chain CDR3
CASGGGADGLTF

SEQ ID NO: 52 - protein, 15 aa, Artificial Sequence
  Synthetic sequence clone15_3F10 alpha chain CDR3
CASMYSGGGADGLTF

SEQ ID NO: 53 - protein, 12 aa, Artificial Sequence
  Synthetic sequence clone17_9B5 alpha chain CDR3
CAGGGGADGLTF

SEQ ID NO: 54 - protein, 17 aa, Artificial Sequence
  Synthetic sequence clone19_5C2 alpha chain CDR3
CAYRSGSDGGSQGNLIF

SEQ ID NO: 55 - protein, 13 aa, Artificial Sequence
  Synthetic sequence clone21_7A10 alpha chain CDR3
CAVRDDYGQNFVF

SEQ ID NO: 56 - protein, 16 aa, Artificial Sequence
  Synthetic sequence clone2_6B8 beta chain CDR3
CASSELPAGGTNEQFF

SEQ ID NO: 57 - protein, 12 aa, Artificial Sequence
  Synthetic sequence clone3_6C3 beta chain CDR3
CASMDLAFKQYF

SEQ ID NO: 58 - protein, 12 aa, Artificial Sequence
  Synthetic sequence clone6_5G8 beta chain CDR3
CASSFLTDTQYF

SEQ ID NO: 59 - protein, 14 aa, Artificial Sequence
  Synthetic sequence clone15_3F10 beta chain CDR3
CASSFFSNTGELFF

SEQ ID NO: 60 - protein, 12 aa, Artificial Sequence
  Synthetic sequence clone17_9B5 beta chain CDR3
CASSYMGPEAFF

SEQ ID NO: 61 - protein, 13 aa, Artificial Sequence
  Synthetic sequence clone19_5C2 beta chain CDR3
CASSQVSGYEQYF

SEQ ID NO: 62 - protein, 13 aa, Artificial Sequence
  Synthetic sequence clone21_7A10 beta chain CDR3
CASSPQGDNEQFF

SEQ ID NO: 63 - protein, 417 aa, Artificial Sequence
  Synthetic sequence Human 5T4
MPGGCSRGPAAGDGRLRLARLALVLLGWVSSSSPTSSASSFSSSAPFLASAVSAQPPLPDQCPALCECSEAARTVKCVNRNLTEVPTDLPAYVRNLFLTGNQLAVLPAGAFARRPPLAELAALNLSGSRLDEVRAGAFEHLPSLRQLDLSHNPLADLSPFFSGSNASVSAPSPLVELILNHIVPPEDERQNRSFEGMVVAALLAGRALQGLRRLELASNHFLYLPRDVLAQLPSLRHLDLSNNSLVSLTYVSFRNLTHLESLHLEDNALKVLHNGTLAEQLPHIRVFLDNNPWVCDCHMADMVTWLKETEVVQGKDRLTCAYPEKMRNRVLLELNSADLDCDPILPPSLQTSYVFLGIVLALIGAIFLLVLYLNRKGIKKWMHNIRDACRDHMEGYHYRYEINADPRLTNLSSNSDV

SEQ ID NO: 64 - protein, 9 aa, Artificial Sequence
  Synthetic sequence Human 5T4 17-25
RLARLALVL

SEQ ID NO: 65 - DNA, 8305 bp, Artificial Sequence
  Synthetic sequence pRRLSIN-ALS_17_9B5 beta chain2-P2A ALS_17_9B5 alpha chain-aWPRE-PST1
tcgagggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccggccaccatgtctctgggactgctgtgttgcggcgccttcagcctgctgtgggccggacctgtgaatgccggcgtgacccagacccccaagttccgggtgctgaaaaccggccagagcatgaccctgctgtgcgcccaggacatgaaccacgagtacatgtattggtacagacaggaccccggcatgggcctgcggctgatccactattctgtgggcgagggcaccaccgccaagggcgaagtgcctgatggctacaacgtgtcccggctgaagaagcagaacttcctgctgggcctggaaagcgccgctcctagccagaccagcgtgtacttctgcgccagcagctatatgggccccgaggcattctttgggcagggaaccagactgaccgtggtggaagatctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgaggttccggagccacgaacttctctctgttaaagcaagcaggagacgtggaagaaaaccccggtcccatggcctgccccggatttctgtgggccctcgtgatcagcacctgtctggaattcagcatggcccagaccgtgacccagagccagcctgagatgagcgtgcaggaagccgagacagtgaccctgagctgcacctacgacaccagcgagagcgactactacctgttctggtacaagcagccccccagccggcagatgatcctcgtgattagacaggaagcctataagcagcagaacgccaccgagaacagattcagcgtgaacttccagaaggccgccaagagcttcagcctgaagatcagcgacagccagctgggcgacgccgccatgtatttttgtgctggcggaggcggagccgacggcctgacatttggaaagggcacccacctgatcatccagccctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagccg

SEQ ID NO: 66 - DNA, 66 bp, Artificial Sequence
  Synthetic sequence Porcine teschovirus-1 2A (P2A) peptide
ggttccggagccacgaacttctctctgttaaagcaagcaggagacgtggaagaaaaccccggtccc

SEQ ID NO: 67 - DNA, 66 bp, Artificial Sequence
  Synthetic sequence Porcine teschovirus-1 2A (P2A) peptide
ggaagcggagctactaacttcagcctgctgaagcaggctggagacgtggaggagaaccctggacct

SEQ ID NO: 68 - DNA, 63 bp, Artificial Sequence
  Synthetic sequence Thoseaasigna virus 2A (T2A) peptide
ggaagcggagagggcagaggaagtctgctaacatgcggtgacgtcgaggagaatcctggacct

SEQ ID NO: 69 - DNA, 69 bp, Artificial Sequence
  Synthetic sequence Equine rhinitis A virus (ERAV) 2A (E2A) peptide
ggaagcggacagtgtactaattatgctctcttgaaattggctggagatgttgagagcaaccctggacct

SEQ ID NO: 70 - DNA, 75 bp, Artificial Sequence
  Synthetic sequence Foot-and-Mouth disease virus 2A (F2A) peptide
ggaagcggagtgaaacagactttgaattttgaccttctcaagttggcgggagacgtggagtccaaccctggacct

SEQ ID NO: 71 - protein, 22 aa, Artificial Sequence
  Synthetic sequence Porcine teschovirus-1 2A (P2A) peptide
GSGATNFSLLKQAGDVEENPGP

SEQ ID NO: 72 - protein, 24 aa, Artificial Sequence
  Thoseaasigna virus 2A (T2A) peptide
LEGGGEGRGSLLTCGDVEENPGPR

SEQ ID NO: 73 - protein, 20 aa, Artificial Sequence
  Synthetic sequence Equine rhinitis A virus (ERAV) 2A (E2A) peptide
QCTNYALLKLAGDVESNPGP

SEQ ID NO: 74 - protein, 25 aa, Artificial Sequence
  Synthetic sequence Foot-and-Mouth disease virus 2A (F2A) peptide
GSGVKQTLNFDLLKLAGDVESNPGP

SEQ ID NO: 75 - protein, 15 aa, Artificial Sequence
  Synthetic sequence Glycine-Serine linker
GGGGSGGGGSGGGGS

SEQ ID NO: 76 - protein, 18 aa, Artificial Sequence
  Synthetic sequence Glycine-Serine linker
GSTSGGGSGGGSGGGGSS

SEQ ID NO: 77 - protein, 274 aa, Artificial Sequence
  Synthetic sequence 2_6B8 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVPDDAGNMLTFGGGTRLMVKPHDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 78 - protein, 275 aa, Artificial Sequence
  Synthetic sequence clone3_6C3 alpha chain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCSSGGGADGLTFGKGTHLIIQPYDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 79 - protein, 275 aa, Artificial Sequence
  Synthetic sequence clone6_5G8 alpha chain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCASGGGADGLTFGKGTHLIIQPYDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 80 - protein, 278 aa, Artificial Sequence
  Synthetic sequence clone15_3F10 alpha chain
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCASMYSGGGADGLTFGKGTHLIIQPYDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 81 - protein, 275 aa, Artificial Sequence
  Synthetic sequence clone17_9B5 alpha chain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAGGGGADGLTFGKGTHLIIQPYDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 82 - protein, 280 aa, Artificial Sequence
  Synthetic sequence clone19_5C2 alpha chain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSGSDGGSQGNLIFGKGTKLSVKPNDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 83 - protein, 269 aa, Artificial Sequence
  Synthetic sequence clone21_7A10 alpha chain
MWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAVRDDYGQNFVFGPGTRLSVLPYDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 84 - protein, 310 aa, Artificial Sequence
  Synthetic sequence 2_6B8 beta chain1
MGTRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYFCASSELPAGGTNEQFFGPGTRLTVLDLKNVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 85 - protein, 307 aa, Artificial Sequence
  Synthetic sequence clone3_6C3 beta chain1
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASMDLAFKQYFGPGTRLTVTEDLKNVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 86 - protein, 307 aa, Artificial Sequence
  Synthetic sequence clone6_5G8 beta chain1
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSFLTDTQYFGPGTRLTVLEDLKNVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 87 - protein, 309 aa, Artificial Sequence
  Synthetic sequence clone15_3F10 beta chain1
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSFFSNTGELFFGEGSRLTVLEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 88 - protein, 307 aa, Artificial Sequence
  Synthetic sequence clone17_9B5 beta chain1
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYMGPEAFFGQGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 89 - protein, 311 aa, Artificial Sequence
  Synthetic sequence clone19_5C2 beta chain1
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQVSGYEQYFGPGTRLTVTEDLKNVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 90 - protein, 309 aa, Artificial Sequence
  Synthetic sequence clone21_7A10 beta chain1
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPQGDNEQFFGPGTRLTVLEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 91 - protein, 312 aa, Artificial Sequence
  Synthetic sequence 2_6B8 beta chain2
MGTRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYFCASSELPAGGTNEQFFGPGTRLTVLDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 92 - protein, 309 aa, Artificial Sequence
  Synthetic sequence clone3_6C3 beta chain2
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASMDLAFKQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 93 - protein, 309 aa, Artificial Sequence
  Synthetic sequence clone6_5G8 beta chain2
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSFLTDTQYFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 94 - protein, 311 aa, Artificial Sequence
  Synthetic sequence clone15_3F10 beta chain2
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSFFSNTGELFFGEGSRLTVLEDLNKVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 95 - protein, 309 aa, Artificial Sequence
  Synthetic sequence clone17_9B5 beta chain2
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYMGPEAFFGQGTRLTVVEDLNKVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 96 - protein, 313 aa, Artificial Sequence
  Synthetic sequence clone19_5C2 beta chain2
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQVSGYEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 97 - protein, 311 aa, Artificial Sequence
  Synthetic sequence clone21_7A10 beta chain2
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPQGDNEQFFGPGTRLTVLEDLNKVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 98 - protein, 606 aa, Artificial Sequence
  Synthetic sequence 2_6B8 beta chain1 P2A 1307_2_6B8 alpha chain
MGTRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYFCASSELPAGGTNEQFFGPGTRLTVLDLKNVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVPDDAGNMLTFGGGTRLMVKPHDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 99 - protein, 604 aa, Artificial Sequence
  Synthetic sequence 3_6C3 beta chain1 P2A ALS_3_6C3 alpha chain
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASMDLAFKQYFGPGTRLTVTEDLKNVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCSSGGGADGLTFGKGTHLIIQPYDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 100 - protein, 604 aa, Artificial Sequence
  Synthetic sequence 6_5G8 beta chain1 P2A DLG_6_5G8 alpha chain
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSFLTDTQYFGPGTRLTVLEDLKNVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCASGGGADGLTFGKGTHLIIQPYDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 101 - protein, 609 aa, Artificial Sequence
  Synthetic sequence 15_3F10 beta chain1 P2A 1307_15_3F10 alpha chain
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSFFSNTGELFFGEGSRLTVLEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCASMYSGGGADGLTFGKGTHLIIQPYDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 102 - protein, 604 aa, Artificial Sequence
  Synthetic sequence 17_9B5 beta chain1 P2A ALS_17_9B5 alpha chain
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYMGPEAFFGQGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAGGGGADGLTFGKGTHLIIQPYDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 103 - protein, 613 aa, Artificial Sequence
  Synthetic sequence 19_5C2 beta chain1 P2A CAD_19_5C2 alpha chain
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQVSGYEQYFGPGTRLTVTEDLKNVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSGSDGGSQGNLIFGKGTKLSVKPNDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 104 - protein, 600 aa, Artificial Sequence
  Synthetic sequence 21_7A10 beta chain1 P2A CAD_21_7A10 alpha chain
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPQGDNEQFFGPGTRLTVLEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAVRDDYGQNFVFGPGTRLSVLPYDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 105 - protein, 608 aa, Artificial Sequence
  Synthetic sequence 2_6B8 beta chain2 P2A 1307_2_6B8 alpha chain
MGTRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYFCASSELPAGGTNEQFFGPGTRLTVLDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVPDDAGNMLTFGGGTRLMVKPHDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 106 - protein, 606 aa, Artificial Sequence
  Synthetic sequence 3_6C3 beta chain2 P2A ALS_3_6C3 alpha chain
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASMDLAFKQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCSSGGGADGLTFGKGTHLIIQPYDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 107 - protein, 606 aa, Artificial Sequence
  Synthetic sequence 6_5G8 beta chain2 P2A DLG_6_5G8 alpha chain
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSFLTDTQYFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCASGGGADGLTFGKGTHLIIQPYDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 108 - protein, 611 aa, Artificial Sequence
  Synthetic sequence 15_3F10 beta chain2 P2A 1307_15_3F10 alpha chain
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSFFSNTGELFFGEGSRLTVLEDLNKVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCASMYSGGGADGLTFGKGTHLIIQPYDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 109 - protein, 606 aa, Artificial Sequence
  Synthetic sequence 17_9B5 beta chain2 P2A ALS_17_9B5 alpha chain
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYMGPEAFFGQGTRLTVVEDLNKVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAGGGGADGLTFGKGTHLIIQPYDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 110 - protein, 615 aa, Artificial Sequence
  Synthetic sequence 19_5C2 beta chain2 P2A CAD_19_5C2 alpha chain
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQVSGYEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSGSDGGSQGNLIFGKGTKLSVKPNDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 111 - protein, 602 aa, Artificial Sequence
  Synthetic sequence 21_7A10 beta chain2 P2A CAD_21_7A10 alpha chain
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPQGDNEQFFGPGTRLTVLEDLNKVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAVRDDYGQNFVFGPGTRLSVLPYDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 112 - DNA, 823 bp, Artificial Sequence
  Synthetic sequence 2_6B8 alpha chainn
atgaagtccctgcgggtgctgctcgtgatcctgtggctccagctgagctgggtgtggtcccagcagaaagaggtggaacagaacagcggccctctgagcgtgccagaaggcgctatcgccagcctgaactgcacctacagcgacagaggcagccagagcttcttctggtacagacagtacagcggcaagagccccgagctgatcatgttcatctacagcaacggcgacaaagaggacggccggttcaccgcccagctgaacaaggccagccagtacgtgtccctgctgatcagagacagccagcccagcgacagcgccacctatctgtgtgccgtgcctgacgacgccggcaacatgctgacatttggcggcggaacccggctgatggtcaagccccgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 113 - DNA, 826 bp, Artificial Sequence
  Synthetic sequence clone3_6C3 alpha chain
atggcctgccccggatttctgtgggccctcgtgatcagcacctgtctggaattcagcatggcccagaccgtgacccagagccagcctgagatgagcgtgcaggaagccgagacagtgaccctgagctgcacctacgacaccagcgagagcgactactacctgttctggtacaagcagccccccagccggcagatgatcctcgtgattagacaggaagcctataagcagcagaacgccaccgagaacagattcagcgtgaacttccagaaggccgccaagagcttcagcctgaagatcagcgacagccagctgggcgacgccgccatgtacttttgttctagcggcggaggcgccgacggcctgacatttggaaagggcacccacctgatcatccagccctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 114 - DNA, 826 bp, Artificial Sequence
  Synthetic sequence clone6_5G8 alpha chain
atggcctgccccggatttctgtgggccctcgtgatcagcacctgtctggaattcagcatggcccagaccgtgacccagagccagcctgagatgagcgtgcaggaagccgagacagtgaccctgagctgcacctacgacaccagcgagagcgactactacctgttctggtacaagcagccccccagccggcagatgatcctcgtgattagacaggaagcctataagcagcagaacgccaccgagaacagattcagcgtgaacttccagaaggccgccaagagcttcagcctgaagatcagcgacagccagctgggcgacgccgccatgtacttttgtgctagcggaggcggagccgacggcctgacatttggcaagggcacccacctgatcatccagccctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 115 - DNA, 835 bp, Artificial Sequence
  Synthetic sequence clone15_3F10 alpha chain
atgacacgagttagcttgctgtgggcagtcgtggtctccacctgtcttgaatccggcatggcccagacagtcactcagtctcaaccagagatgtctgtgcaggaggcagagactgtgaccctgagttgcacatatgacaccagtgagaataattattatttgttctggtacaagcagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgcctcgatgtattcaggaggaggtgctgacggactcacctttggcaaagggactcatctaatcatccagccctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 116 - DNA, 826 bp, Artificial Sequence
  Synthetic sequence clone17_9B5 alpha chain
atggcctgccccggatttctgtgggccctcgtgatcagcacctgtctggaattcagcatggcccagaccgtgacccagagccagcctgagatgagcgtgcaggaagccgagacagtgaccctgagctgcacctacgacaccagcgagagcgactactacctgttctggtacaagcagccccccagccggcagatgatcctcgtgattagacaggaagcctataagcagcagaacgccaccgagaacagattcagcgtgaacttccagaaggccgccaagagcttcagcctgaagatcagcgacagccagctgggcgacgccgccatgtatttttgtgctggcggaggcggagccgacggcctgacatttggaaagggcacccacctgatcatccagccctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 117 - DNA, 841 bp, Artificial Sequence
  Synthetic sequence clone19_5C2 alpha chain
atggcctgccccggatttctgtgggccctcgtgatcagcacctgtctggaattcagcatggcccagaccgtgacccagagccagcctgagatgagcgtgcaggaagccgagacagtgaccctgagctgcacctacgacaccagcgagagcgactactacctgttctggtacaagcagccccccagccggcagatgatcctcgtgattagacaggaagcctataagcagcagaacgccaccgagaacagattcagcgtgaacttccagaaggccgccaagagcttcagcctgaagatcagcgacagccagctgggcgacgccgccatgtacttttgcgcctatagaagcggcagcgacggcggctctcagggcaatctgatcttcggcaagggcaccaagctgagcgtgaagcccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 118 - DNA, 808 bp, Artificial Sequence
  Synthetic sequence clone21_7A10 alpha chain
atgtggggcgcctttctgctgtacgtgtccatgaagatgggcggcacagccggccagagcctggaacagccttctgaagtgaccgccgtggaaggcgccatcgtgcagatcaactgcacctaccagaccagcggcttctacggcctgagctggtatcagcagcatgacggcggagcccccaccttcctgagctacaatgccctggacggcctggaagagacaggccggttcagcagcttcctgtccagaagcgacagctacggctacctgctgctccaggaactccagatgaaggacagcgccagctacttctgcgccgtgcgggatgactacggccagaacttcgtgttcggccctggcaccagactgagcgtgctgccctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 119 - DNA, 933 bp, Artificial Sequence
  Synthetic sequence 2_6B8 beta chain1
atgggcaccagactgtttttctacgtggccctgtgcctgctgtgggccggacatagagatgccggaatcacccagagcccccggtacaagatcaccgagacaggcagacaagtgaccctgatgtgccaccagacctggtcccacagctacatgttctggtacagacaggacctgggccacggcctgcggctgatctactattctgccgccgctgacatcaccgacaagggcgaagtgcccgacggctacgtggtgtccagaagcaagaccgagaacttcccactgaccctggaaagcgccacccggtcccagaccagcgtgtacttttgtgccagcagcgagctgcccgctggcggcaccaatgagcagttttttggccctggcacccggctgaccgtgctggacctgaagaatgtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 120 - DNA, 924 bp, Artificial Sequence
  Synthetic sequence clone3_6C3 beta chain1
atgtctctgggactgctgtgttgcggcgccttcagcctgctgtgggccggacctgtgaatgccggcgtgacccagacccccaagttccgggtgctgaaaaccggccagagcatgaccctgctgtgcgcccaggacatgaaccacgagtacatgtattggtacagacaggaccccggcatgggcctgcggctgatccactattctgtgggcgagggcaccaccgccaagggcgaagtgcctgatggctacaacgtgtcccggctgaagaagcagaacttcctgctgggcctggaaagcgccgctcctagccagaccagcgtgtacttctgcgccagcatggacctggccttcaagcagtacttcggccctggcaccagactgaccgtgaccgaggacctgaagaacgtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 121 - DNA, 924 bp, Artificial Sequence
  Synthetic sequence clone6_5G8 beta chain1
atgtctctgggactgctgtgttgcggcgccttcagcctgctgtgggccggacctgtgaatgccggcgtgacccagacccccaagttccgggtgctgaaaaccggccagagcatgaccctgctgtgcgcccaggacatgaaccacgagtacatgtattggtacagacaggaccccggcatgggcctgcggctgatccactattctgtgggcgagggcaccaccgccaagggcgaagtgcctgatggctacaacgtgtcccggctgaagaagcagaacttcctgctgggcctggaaagcgccgctcctagccagaccagcgtgtacttctgcgccagcagcttcctgaccgacacccagtactttggccctggcaccagactgaccgtgctggaagatctgaagaacgtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 122 - DNA, 930 bp, Artificial Sequence
  Synthetic sequence clone15_3F10 beta chain1
atgggacctcagctgctgggatacgtggtgctgtgtctgctgggagccggacctctggaagcccaagtgacccagaaccccagatacctgatcaccgtgaccggcaagaaactgaccgtgacctgctcccagaacatgaaccacgagtacatgagctggtacagacaggaccccggcctgggcctgcggcagatctactacagcatgaacgtggaagtgaccgacaagggcgacgtgcccgagggctacaaggtgtcccggaaagagaagcggaacttcccactgatcctggaaagccccagccccaaccagaccagcctgtacttctgcgccagcagcttcttcagcaacaccggcgagctgttcttcggcgagggcagcagactgacagtgctggaagatctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 123 - DNA, 924 bp, Artificial Sequence
  Synthetic sequence clone17_9B5 beta chain1
atgtctctgggactgctgtgttgcggcgccttcagcctgctgtgggccggacctgtgaatgccggcgtgacccagacccccaagttccgggtgctgaaaaccggccagagcatgaccctgctgtgcgcccaggacatgaaccacgagtacatgtattggtacagacaggaccccggcatgggcctgcggctgatccactattctgtgggcgagggcaccaccgccaagggcgaagtgcctgatggctacaacgtgtcccggctgaagaagcagaacttcctgctgggcctggaaagcgccgctcctagccagaccagcgtgtacttctgcgccagcagctatatgggccccgaggcattctttgggcagggaaccagactgaccgtggtggaagatctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 124 - DNA, 936 bp, Artificial Sequence
  Synthetic sequence clone19_5C2 beta chain1
atgggatgtagactgctgtgttgcgccgtgctgtgcctgctgggagctggcgaactggtgcctatggaaaccggcgtgacccagacccccagacacctcgtgatgggcatgaccaacaagaaaagcctgaagtgcgagcagcacctgggccacaacgccatgtactggtacaagcagagcgccaagaaacccctggaactgatgttcgtgtacagcctggaagagagggtggaaaacaacagcgtgcccagccggttcagccccgagtgccctaatagcagccacctgtttctgcatctgcacaccctccagcccgaggacagcgccctgtatctgtgtgccagctctcaggtgtccggctacgagcagtacttcggccctggcaccagactgaccgtgaccgaggacctgaagaacgtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 125 - DNA, 930 bp, Artificial Sequence
  Synthetic sequence clone21_7A10 beta chain1
atggatacaagagtgctgtgctgcgccgtgatctgcctgctgggagccggactgtctaatgccggcgtgatgcagaaccccagacacctcgtgcggcggagaggacaggaagccagactgcgctgtagccccatgaagggccacagccacgtgtactggtacagacagctgcccgaagagggcctgaagttcatggtgtacctccagaaagagaacatcatcgacgagagcggcatgcccaaagagcggttcagcgccgagttccccaaagagggccccagcatcctgagaatccagcaggtcgtgcggggcgatagcgccgcctatttttgtgccagcagcccccagggcgacaacgagcagttttttggccctggcacccggctgaccgtgctggaagatctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 126 - DNA, 939 bp, Artificial Sequence
  Synthetic sequence 2_6B8 beta chain2
atgggcaccagactgtttttctacgtggccctgtgcctgctgtgggccggacatagagatgccggaatcacccagagcccccggtacaagatcaccgagacaggcagacaagtgaccctgatgtgccaccagacctggtcccacagctacatgttctggtacagacaggacctgggccacggcctgcggctgatctactattctgccgccgctgacatcaccgacaagggcgaagtgcccgacggctacgtggtgtccagaagcaagaccgagaacttcccactgaccctggaaagcgccacccggtcccagaccagcgtgtacttttgtgccagcagcgagctgcccgctggcggcaccaatgagcagttttttggccctggcacccggctgaccgtgctggacctgaagaatgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 127 - DNA, 930 bp, Artificial Sequence
  Synthetic sequence clone3_6C3 beta chain2
atgtctctgggactgctgtgttgcggcgccttcagcctgctgtgggccggacctgtgaatgccggcgtgacccagacccccaagttccgggtgctgaaaaccggccagagcatgaccctgctgtgcgcccaggacatgaaccacgagtacatgtattggtacagacaggaccccggcatgggcctgcggctgatccactattctgtgggcgagggcaccaccgccaagggcgaagtgcctgatggctacaacgtgtcccggctgaagaagcagaacttcctgctgggcctggaaagcgccgctcctagccagaccagcgtgtacttctgcgccagcatggacctggccttcaagcagtacttcggccctggcaccagactgaccgtgaccgaggacctgaagaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 128 - DNA, 930 bp, Artificial Sequence
  Synthetic sequence clone6_5G8 beta chain2
atgtctctgggactgctgtgttgcggcgccttcagcctgctgtgggccggacctgtgaatgccggcgtgacccagacccccaagttccgggtgctgaaaaccggccagagcatgaccctgctgtgcgcccaggacatgaaccacgagtacatgtattggtacagacaggaccccggcatgggcctgcggctgatccactattctgtgggcgagggcaccaccgccaagggcgaagtgcctgatggctacaacgtgtcccggctgaagaagcagaacttcctgctgggcctggaaagcgccgctcctagccagaccagcgtgtacttctgcgccagcagcttcctgaccgacacccagtactttggccctggcaccagactgaccgtgctggaagatctgaagaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 129 - DNA, 936 bp, Artificial Sequence
  Synthetic sequence clone15_3F10 beta chain2
atgggacctcagctgctgggatacgtggtgctgtgtctgctgggagccggacctctggaagcccaagtgacccagaaccccagatacctgatcaccgtgaccggcaagaaactgaccgtgacctgctcccagaacatgaaccacgagtacatgagctggtacagacaggaccccggcctgggcctgcggcagatctactacagcatgaacgtggaagtgaccgacaagggcgacgtgcccgagggctacaaggtgtcccggaaagagaagcggaacttcccactgatcctggaaagccccagccccaaccagaccagcctgtacttctgcgccagcagcttcttcagcaacaccggcgagctgttcttcggcgagggcagcagactgacagtgctggaagatctgaacaaggtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 130 - DNA, 930 bp, Artificial Sequence
  Synthetic sequence clone17_9B5 beta chain2
atgtctctgggactgctgtgttgcggcgccttcagcctgctgtgggccggacctgtgaatgccggcgtgacccagacccccaagttccgggtgctgaaaaccggccagagcatgaccctgctgtgcgcccaggacatgaaccacgagtacatgtattggtacagacaggaccccggcatgggcctgcggctgatccactattctgtgggcgagggcaccaccgccaagggcgaagtgcctgatggctacaacgtgtcccggctgaagaagcagaacttcctgctgggcctggaaagcgccgctcctagccagaccagcgtgtacttctgcgccagcagctatatgggccccgaggcattctttgggcagggaaccagactgaccgtggtggaagatctgaacaaggtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 131 - DNA, 942 bp, Artificial Sequence
  Synthetic sequence clone19_5C2 beta chain2
atgggatgtagactgctgtgttgcgccgtgctgtgcctgctgggagctggcgaactggtgcctatggaaaccggcgtgacccagacccccagacacctcgtgatgggcatgaccaacaagaaaagcctgaagtgcgagcagcacctgggccacaacgccatgtactggtacaagcagagcgccaagaaacccctggaactgatgttcgtgtacagcctggaagagagggtggaaaacaacagcgtgcccagccggttcagccccgagtgccctaatagcagccacctgtttctgcatctgcacaccctccagcccgaggacagcgccctgtatctgtgtgccagctctcaggtgtccggctacgagcagtacttcggccctggcaccagactgaccgtgaccgaggacctgaagaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 132 - DNA, 936 bp, Artificial Sequence
  Synthetic sequence clone21_7A10 beta chain2
atggatacaagagtgctgtgctgcgccgtgatctgcctgctgggagccggactgtctaatgccggcgtgatgcagaaccccagacacctcgtgcggcggagaggacaggaagccagactgcgctgtagccccatgaagggccacagccacgtgtactggtacagacagctgcccgaagagggcctgaagttcatggtgtacctccagaaagagaacatcatcgacgagagcggcatgcccaaagagcggttcagcgccgagttccccaaagagggccccagcatcctgagaatccagcaggtcgtgcggggcgatagcgccgcctatttttgtgccagcagcccccagggcgacaacgagcagttttttggccctggcacccggctgaccgtgctggaagatctgaacaaggtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 133 - DNA, 1822 bp, Artificial Sequence
  Synthetic sequence 2_6B8 beta chain1 P2A 1307_2_6B8 alpha chain
atgggcaccagactgtttttctacgtggccctgtgcctgctgtgggccggacatagagatgccggaatcacccagagcccccggtacaagatcaccgagacaggcagacaagtgaccctgatgtgccaccagacctggtcccacagctacatgttctggtacagacaggacctgggccacggcctgcggctgatctactattctgccgccgctgacatcaccgacaagggcgaagtgcccgacggctacgtggtgtccagaagcaagaccgagaacttcccactgaccctggaaagcgccacccggtcccagaccagcgtgtacttttgtgccagcagcgagctgcccgctggcggcaccaatgagcagttttttggccctggcacccggctgaccgtgctggacctgaagaatgtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgaggttccggagccacgaacttctctctgttaaagcaagcaggagacgtggaagaaaaccccggtcccatgaagtccctgcgggtgctgctcgtgatcctgtggctccagctgagctgggtgtggtcccagcagaaagaggtggaacagaacagcggccctctgagcgtgccagaaggcgctatcgccagcctgaactgcacctacagcgacagaggcagccagagcttcttctggtacagacagtacagcggcaagagccccgagctgatcatgttcatctacagcaacggcgacaaagaggacggccggttcaccgcccagctgaacaaggccagccagtacgtgtccctgctgatcagagacagccagcccagcgacagcgccacctatctgtgtgccgtgcctgacgacgccggcaacatgctgacatttggcggcggaacccggctgatggtcaagccccgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 134 - DNA, 1816 bp, Artificial Sequence
  Synthetic sequence 3_6C3 beta chain1 P2A ALS_3_6C3 alpha chain
atgtctctgggactgctgtgttgcggcgccttcagcctgctgtgggccggacctgtgaatgccggcgtgacccagacccccaagttccgggtgctgaaaaccggccagagcatgaccctgctgtgcgcccaggacatgaaccacgagtacatgtattggtacagacaggaccccggcatgggcctgcggctgatccactattctgtgggcgagggcaccaccgccaagggcgaagtgcctgatggctacaacgtgtcccggctgaagaagcagaacttcctgctgggcctggaaagcgccgctcctagccagaccagcgtgtacttctgcgccagcatggacctggccttcaagcagtacttcggccctggcaccagactgaccgtgaccgaggacctgaagaacgtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgaggttccggagccacgaacttctctctgttaaagcaagcaggagacgtggaagaaaaccccggtcccatggcctgccccggatttctgtgggccctcgtgatcagcacctgtctggaattcagcatggcccagaccgtgacccagagccagcctgagatgagcgtgcaggaagccgagacagtgaccctgagctgcacctacgacaccagcgagagcgactactacctgttctggtacaagcagccccccagccggcagatgatcctcgtgattagacaggaagcctataagcagcagaacgccaccgagaacagattcagcgtgaacttccagaaggccgccaagagcttcagcctgaagatcagcgacagccagctgggcgacgccgccatgtacttttgttctagcggcggaggcgccgacggcctgacatttggaaagggcacccacctgatcatccagccctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 135 - DNA, 1816 bp, Artificial Sequence
  Synthetic sequence 6_5G8 beta chain1 P2A DLG_6_5G8 alpha chain
atgtctctgggactgctgtgttgcggcgccttcagcctgctgtgggccggacctgtgaatgccggcgtgacccagacccccaagttccgggtgctgaaaaccggccagagcatgaccctgctgtgcgcccaggacatgaaccacgagtacatgtattggtacagacaggaccccggcatgggcctgcggctgatccactattctgtgggcgagggcaccaccgccaagggcgaagtgcctgatggctacaacgtgtcccggctgaagaagcagaacttcctgctgggcctggaaagcgccgctcctagccagaccagcgtgtacttctgcgccagcagcttcctgaccgacacccagtactttggccctggcaccagactgaccgtgctggaagatctgaagaacgtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgaggttccggagccacgaacttctctctgttaaagcaagcaggagacgtggaagaaaaccccggtcccatggcctgccccggatttctgtgggccctcgtgatcagcacctgtctggaattcagcatggcccagaccgtgacccagagccagcctgagatgagcgtgcaggaagccgagacagtgaccctgagctgcacctacgacaccagcgagagcgactactacctgttctggtacaagcagccccccagccggcagatgatcctcgtgattagacaggaagcctataagcagcagaacgccaccgagaacagattcagcgtgaacttccagaaggccgccaagagcttcagcctgaagatcagcgacagccagctgggcgacgccgccatgtacttttgtgctagcggaggcggagccgacggcctgacatttggcaagggcacccacctgatcatccagccctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 136 - DNA, 1831 bp, Artificial Sequence
  Synthetic sequence 15_3F10 beta chain1 P2A 1307_15_3F10 alpha chain
atgggacctcagctgctgggatacgtggtgctgtgtctgctgggagccggacctctggaagcccaagtgacccagaaccccagatacctgatcaccgtgaccggcaagaaactgaccgtgacctgctcccagaacatgaaccacgagtacatgagctggtacagacaggaccccggcctgggcctgcggcagatctactacagcatgaacgtggaagtgaccgacaagggcgacgtgcccgagggctacaaggtgtcccggaaagagaagcggaacttcccactgatcctggaaagccccagccccaaccagaccagcctgtacttctgcgccagcagcttcttcagcaacaccggcgagctgttcttcggcgagggcagcagactgacagtgctggaagatctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgaggttccggagccacgaacttctctctgttaaagcaagcaggagacgtggaagaaaaccccggtcccatgacacgagttagcttgctgtgggcagtcgtggtctccacctgtcttgaatccggcatggcccagacagtcactcagtctcaaccagagatgtctgtgcaggaggcagagactgtgaccctgagttgcacatatgacaccagtgagaataattattatttgttctggtacaagcagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgcctcgatgtattcaggaggaggtgctgacggactcacctttggcaaagggactcatctaatcatccagccctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 137 - DNA, 1816 bp, Artificial Sequence
  Synthetic sequence 17_9B5 beta chain1 P2A ALS_17_9B5 alpha chain
atgtctctgggactgctgtgttgcggcgccttcagcctgctgtgggccggacctgtgaatgccggcgtgacccagacccccaagttccgggtgctgaaaaccggccagagcatgaccctgctgtgcgcccaggacatgaaccacgagtacatgtattggtacagacaggaccccggcatgggcctgcggctgatccactattctgtgggcgagggcaccaccgccaagggcgaagtgcctgatggctacaacgtgtcccggctgaagaagcagaacttcctgctgggcctggaaagcgccgctcctagccagaccagcgtgtacttctgcgccagcagctatatgggccccgaggcattctttgggcagggaaccagactgaccgtggtggaagatctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgaggttccggagccacgaacttctctctgttaaagcaagcaggagacgtggaagaaaaccccggtcccatggcctgccccggatttctgtgggccctcgtgatcagcacctgtctggaattcagcatggcccagaccgtgacccagagccagcctgagatgagcgtgcaggaagccgagacagtgaccctgagctgcacctacgacaccagcgagagcgactactacctgttctggtacaagcagccccccagccggcagatgatcctcgtgattagacaggaagcctataagcagcagaacgccaccgagaacagattcagcgtgaacttccagaaggccgccaagagcttcagcctgaagatcagcgacagccagctgggcgacgccgccatgtatttttgtgctggcggaggcggagccgacggcctgacatttggaaagggcacccacctgatcatccagccctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 138 - DNA, 1843 bp, Artificial Sequence
  Synthetic sequence 19_5C2 beta chain1 P2A CAD_19_5C2 alpha chain
atgggatgtagactgctgtgttgcgccgtgctgtgcctgctgggagctggcgaactggtgcctatggaaaccggcgtgacccagacccccagacacctcgtgatgggcatgaccaacaagaaaagcctgaagtgcgagcagcacctgggccacaacgccatgtactggtacaagcagagcgccaagaaacccctggaactgatgttcgtgtacagcctggaagagagggtggaaaacaacagcgtgcccagccggttcagccccgagtgccctaatagcagccacctgtttctgcatctgcacaccctccagcccgaggacagcgccctgtatctgtgtgccagctctcaggtgtccggctacgagcagtacttcggccctggcaccagactgaccgtgaccgaggacctgaagaacgtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgaggttccggagccacgaacttctctctgttaaagcaagcaggagacgtggaagaaaaccccggtcccatggcctgccccggatttctgtgggccctcgtgatcagcacctgtctggaattcagcatggcccagaccgtgacccagagccagcctgagatgagcgtgcaggaagccgagacagtgaccctgagctgcacctacgacaccagcgagagcgactactacctgttctggtacaagcagccccccagccggcagatgatcctcgtgattagacaggaagcctataagcagcagaacgccaccgagaacagattcagcgtgaacttccagaaggccgccaagagcttcagcctgaagatcagcgacagccagctgggcgacgccgccatgtacttttgcgcctatagaagcggcagcgacggcggctctcagggcaatctgatcttcggcaagggcaccaagctgagcgtgaagcccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 139 - DNA, 1804 bp, Artificial Sequence
  Synthetic sequence 21_7A10 beta chain1 P2A CAD_21_7A10 alpha chain
atggatacaagagtgctgtgctgcgccgtgatctgcctgctgggagccggactgtctaatgccggcgtgatgcagaaccccagacacctcgtgcggcggagaggacaggaagccagactgcgctgtagccccatgaagggccacagccacgtgtactggtacagacagctgcccgaagagggcctgaagttcatggtgtacctccagaaagagaacatcatcgacgagagcggcatgcccaaagagcggttcagcgccgagttccccaaagagggccccagcatcctgagaatccagcaggtcgtgcggggcgatagcgccgcctatttttgtgccagcagcccccagggcgacaacgagcagttttttggccctggcacccggctgaccgtgctggaagatctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgaggttccggagccacgaacttctctctgttaaagcaagcaggagacgtggaagaaaaccccggtcccatgtggggcgcctttctgctgtacgtgtccatgaagatgggcggcacagccggccagagcctggaacagccttctgaagtgaccgccgtggaaggcgccatcgtgcagatcaactgcacctaccagaccagcggcttctacggcctgagctggtatcagcagcatgacggcggagcccccaccttcctgagctacaatgccctggacggcctggaagagacaggccggttcagcagcttcctgtccagaagcgacagctacggctacctgctgctccaggaactccagatgaaggacagcgccagctacttctgcgccgtgcgggatgactacggccagaacttcgtgttcggccctggcaccagactgagcgtgctgccctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 140 - DNA, 1828 bp, Artificial Sequence
  Synthetic sequence 2_6B8 beta chain2 P2A 1307_2_6B8 alpha chain
atgggcaccagactgtttttctacgtggccctgtgcctgctgtgggccggacatagagatgccggaatcacccagagcccccggtacaagatcaccgagacaggcagacaagtgaccctgatgtgccaccagacctggtcccacagctacatgttctggtacagacaggacctgggccacggcctgcggctgatctactattctgccgccgctgacatcaccgacaagggcgaagtgcccgacggctacgtggtgtccagaagcaagaccgagaacttcccactgaccctggaaagcgccacccggtcccagaccagcgtgtacttttgtgccagcagcgagctgcccgctggcggcaccaatgagcagttttttggccctggcacccggctgaccgtgctggacctgaagaatgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctagggttccggagccacgaacttctctctgttaaagcaagcaggagacgtggaagaaaaccccggtcccatgaagtccctgcgggtgctgctcgtgatcctgtggctccagctgagctgggtgtggtcccagcagaaagaggtggaacagaacagcggccctctgagcgtgccagaaggcgctatcgccagcctgaactgcacctacagcgacagaggcagccagagcttcttctggtacagacagtacagcggcaagagccccgagctgatcatgttcatctacagcaacggcgacaaagaggacggccggttcaccgcccagctgaacaaggccagccagtacgtgtccctgctgatcagagacagccagcccagcgacagcgccacctatctgtgtgccgtgcctgacgacgccggcaacatgctgacatttggcggcggaacccggctgatggtcaagccccgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 141 - DNA, 1822 bp, Artificial Sequence
  Synthetic sequence 3_6C3 beta chain2 P2A ALS_3_6C3 alpha chain
atgtctctgggactgctgtgttgcggcgccttcagcctgctgtgggccggacctgtgaatgccggcgtgacccagacccccaagttccgggtgctgaaaaccggccagagcatgaccctgctgtgcgcccaggacatgaaccacgagtacatgtattggtacagacaggaccccggcatgggcctgcggctgatccactattctgtgggcgagggcaccaccgccaagggcgaagtgcctgatggctacaacgtgtcccggctgaagaagcagaacttcctgctgggcctggaaagcgccgctcctagccagaccagcgtgtacttctgcgccagcatggacctggccttcaagcagtacttcggccctggcaccagactgaccgtgaccgaggacctgaagaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctagggttccggagccacgaacttctctctgttaaagcaagcaggagacgtggaagaaaaccccggtcccatggcctgccccggatttctgtgggccctcgtgatcagcacctgtctggaattcagcatggcccagaccgtgacccagagccagcctgagatgagcgtgcaggaagccgagacagtgaccctgagctgcacctacgacaccagcgagagcgactactacctgttctggtacaagcagccccccagccggcagatgatcctcgtgattagacaggaagcctataagcagcagaacgccaccgagaacagattcagcgtgaacttccagaaggccgccaagagcttcagcctgaagatcagcgacagccagctgggcgacgccgccatgtacttttgttctagcggcggaggcgccgacggcctgacatttggaaagggcacccacctgatcatccagccctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 142 - DNA, 1822 bp, Artificial Sequence
  Synthetic sequence 6_5G8 beta chain2 P2A DLG_6_5G8 alpha chain
atgtctctgggactgctgtgttgcggcgccttcagcctgctgtgggccggacctgtgaatgccggcgtgacccagacccccaagttccgggtgctgaaaaccggccagagcatgaccctgctgtgcgcccaggacatgaaccacgagtacatgtattggtacagacaggaccccggcatgggcctgcggctgatccactattctgtgggcgagggcaccaccgccaagggcgaagtgcctgatggctacaacgtgtcccggctgaagaagcagaacttcctgctgggcctggaaagcgccgctcctagccagaccagcgtgtacttctgcgccagcagcttcctgaccgacacccagtactttggccctggcaccagactgaccgtgctggaagatctgaagaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctagggttccggagccacgaacttctctctgttaaagcaagcaggagacgtggaagaaaaccccggtcccatggcctgccccggatttctgtgggccctcgtgatcagcacctgtctggaattcagcatggcccagaccgtgacccagagccagcctgagatgagcgtgcaggaagccgagacagtgaccctgagctgcacctacgacaccagcgagagcgactactacctgttctggtacaagcagccccccagccggcagatgatcctcgtgattagacaggaagcctataagcagcagaacgccaccgagaacagattcagcgtgaacttccagaaggccgccaagagcttcagcctgaagatcagcgacagccagctgggcgacgccgccatgtacttttgtgctagcggaggcggagccgacggcctgacatttggcaagggcacccacctgatcatccagccctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 143 - DNA, 1837 bp, Artificial Sequence
  Synthetic sequence 15_3F10 beta chain2 P2A 1307_15_3F10 alpha chain
atgggacctcagctgctgggatacgtggtgctgtgtctgctgggagccggacctctggaagcccaagtgacccagaaccccagatacctgatcaccgtgaccggcaagaaactgaccgtgacctgctcccagaacatgaaccacgagtacatgagctggtacagacaggaccccggcctgggcctgcggcagatctactacagcatgaacgtggaagtgaccgacaagggcgacgtgcccgagggctacaaggtgtcccggaaagagaagcggaacttcccactgatcctggaaagccccagccccaaccagaccagcctgtacttctgcgccagcagcttcttcagcaacaccggcgagctgttcttcggcgagggcagcagactgacagtgctggaagatctgaacaaggtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctagggttccggagccacgaacttctctctgttaaagcaagcaggagacgtggaagaaaaccccggtcccatgacacgagttagcttgctgtgggcagtcgtggtctccacctgtcttgaatccggcatggcccagacagtcactcagtctcaaccagagatgtctgtgcaggaggcagagactgtgaccctgagttgcacatatgacaccagtgagaataattattatttgttctggtacaagcagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgcctcgatgtattcaggaggaggtgctgacggactcacctttggcaaagggactcatctaatcatccagccctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 144 - DNA, 1822 bp, Artificial Sequence
  Synthetic sequence 17_9B5 beta chain2 P2A ALS_17_9B5 alpha chain
atgtctctgggactgctgtgttgcggcgccttcagcctgctgtgggccggacctgtgaatgccggcgtgacccagacccccaagttccgggtgctgaaaaccggccagagcatgaccctgctgtgcgcccaggacatgaaccacgagtacatgtattggtacagacaggaccccggcatgggcctgcggctgatccactattctgtgggcgagggcaccaccgccaagggcgaagtgcctgatggctacaacgtgtcccggctgaagaagcagaacttcctgctgggcctggaaagcgccgctcctagccagaccagcgtgtacttctgcgccagcagctatatgggccccgaggcattctttgggcagggaaccagactgaccgtggtggaagatctgaacaaggtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctagggttccggagccacgaacttctctctgttaaagcaagcaggagacgtggaagaaaaccccggtcccatggcctgccccggatttctgtgggccctcgtgatcagcacctgtctggaattcagcatggcccagaccgtgacccagagccagcctgagatgagcgtgcaggaagccgagacagtgaccctgagctgcacctacgacaccagcgagagcgactactacctgttctggtacaagcagccccccagccggcagatgatcctcgtgattagacaggaagcctataagcagcagaacgccaccgagaacagattcagcgtgaacttccagaaggccgccaagagcttcagcctgaagatcagcgacagccagctgggcgacgccgccatgtatttttgtgctggcggaggcggagccgacggcctgacatttggaaagggcacccacctgatcatccagccctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 145 - DNA, 1849 bp, Artificial Sequence
  Synthetic sequence 19_5C2 beta chain2 P2A CAD_19_5C2 alpha chain
atgggatgtagactgctgtgttgcgccgtgctgtgcctgctgggagctggcgaactggtgcctatggaaaccggcgtgacccagacccccagacacctcgtgatgggcatgaccaacaagaaaagcctgaagtgcgagcagcacctgggccacaacgccatgtactggtacaagcagagcgccaagaaacccctggaactgatgttcgtgtacagcctggaagagagggtggaaaacaacagcgtgcccagccggttcagccccgagtgccctaatagcagccacctgtttctgcatctgcacaccctccagcccgaggacagcgccctgtatctgtgtgccagctctcaggtgtccggctacgagcagtacttcggccctggcaccagactgaccgtgaccgaggacctgaagaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctagggttccggagccacgaacttctctctgttaaagcaagcaggagacgtggaagaaaaccccggtcccatggcctgccccggatttctgtgggccctcgtgatcagcacctgtctggaattcagcatggcccagaccgtgacccagagccagcctgagatgagcgtgcaggaagccgagacagtgaccctgagctgcacctacgacaccagcgagagcgactactacctgttctggtacaagcagccccccagccggcagatgatcctcgtgattagacaggaagcctataagcagcagaacgccaccgagaacagattcagcgtgaacttccagaaggccgccaagagcttcagcctgaagatcagcgacagccagctgggcgacgccgccatgtacttttgcgcctatagaagcggcagcgacggcggctctcagggcaatctgatcttcggcaagggcaccaagctgagcgtgaagcccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 146 - DNA, 1810 bp, Artificial Sequence
  Synthetic sequence 21_7A10 beta chain2 P2A CAD_21_7A10 alpha chain
atggatacaagagtgctgtgctgcgccgtgatctgcctgctgggagccggactgtctaatgccggcgtgatgcagaaccccagacacctcgtgcggcggagaggacaggaagccagactgcgctgtagccccatgaagggccacagccacgtgtactggtacagacagctgcccgaagagggcctgaagttcatggtgtacctccagaaagagaacatcatcgacgagagcggcatgcccaaagagcggttcagcgccgagttccccaaagagggccccagcatcctgagaatccagcaggtcgtgcggggcgatagcgccgcctatttttgtgccagcagcccccagggcgacaacgagcagttttttggccctggcacccggctgaccgtgctggaagatctgaacaaggtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctagggttccggagccacgaacttctctctgttaaagcaagcaggagacgtggaagaaaaccccggtcccatgtggggcgcctttctgctgtacgtgtccatgaagatgggcggcacagccggccagagcctggaacagccttctgaagtgaccgccgtggaaggcgccatcgtgcagatcaactgcacctaccagaccagcggcttctacggcctgagctggtatcagcagcatgacggcggagcccccaccttcctgagctacaatgccctggacggcctggaagagacaggccggttcagcagcttcctgtccagaagcgacagctacggctacctgctgctccaggaactccagatgaaggacagcgccagctacttctgcgccgtgcgggatgactacggccagaacttcgtgttcggccctggcaccagactgagcgtgctgccctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 147 - DNA, 28 bp, Artificial Sequence
  Synthetic sequence TCR Valpha 5′ fragment overhang sequence
aggagacgtggaagaaaaccccggtccc

SEQ ID NO: 148 - DNA, 41 bp, Artificial Sequence
  Synthetic sequence TCR Valpha 3′ fragment overhang sequence
acatccagaaccccgaccctgcagtgtaccagctgcgggac

SEQ ID NO: 149 - DNA, 53 bp, Artificial Sequence
  Synthetic sequence TCR Vbeta 5′ fragment overhang sequence
tccccgagctcaataaaagagcccacaacccctcactcggcgcgccggccacc

SEQ ID NO: 150 - DNA, 30 bp, Artificial Sequence
  Synthetic sequence TCR Vbeta 3′ fragment overhang sequence
gtgttccccccagaggtggccgtgttcgag

SEQ ID NO: 151 - DNA, 25 bp, Artificial Sequence
  Synthetic sequence sgRNA Forward Oligo TRAC_sgRNA_pLenti_F1
caccggagaatcaaaatcggtgaat

SEQ ID NO: 152 - DNA, 25 bp, Artificial Sequence
  Synthetic sequence sgRNA Reverse Oligo TRAC_sgRNA_pLenti_R1
aaacattcaccgattttgattctcc

SEQ ID NO: 153 - DNA, 24 bp, Artificial Sequence
  Synthetic sequence PD1_sgRNA_F1
caccgcagttgtgtgacacggaag

SEQ ID NO: 154 - DNA, 24 bp, Artificial Sequence
  Synthetic sequence PD1_sgRNA_R1
aaaccttccgtgtcacacaactgc

SEQ ID NO: 155 - DNA, 25 bp, Artificial Sequence
  Synthetic sequence CTLA4_sgRNA_F1
caccggcaaaggtgagtgagacttt

SEQ ID NO: 156 - DNA, 25 bp, Artificial Sequence
  Synthetic sequence CTLA4_sgRNA_R1
aaacaaagtctcactcacctttgcc

SEQ ID NO: 157 - DNA, 25 bp, Artificial Sequence
  Synthetic sequence LAG3_sgRNA_F1
caccggtttctgcagccgctttggg

SEQ ID NO: 158 - DNA, 25 bp, Artificial Sequence
  Synthetic sequence LAG3_sgRNA_R1
aaaccccaaagcggctgcagaaacc

SEQ ID NO: 159 - protein, 9 aa, Artificial Sequence
  Synthetic sequence structure motif
LAGLIDADG

SEQ ID NO: 160 - protein, 6 aa, Artificial Sequence
  Synthetic sequence structure motif
GIYYIG

SEQ ID NO: 161 - protein, 5 aa, Artificial Sequence
  Synthetic sequence structure motif
  VARIANT 3: Xaa = Asp or Glu
  VARIANT 4: Xaa = any amino acid
PDXXK

SEQ ID NO: 162 - protein, 9 aa, Artificial Sequence
  Synthetic sequence 5T4HLA complex
RLARLALVL

SEQ ID NO: 163 - protein, 10 aa, Artificial Sequence
  Synthetic linker sequence
GGGGSGGGGS

SEQ ID NO: 164 - protein, 7 aa, Artificial Sequence
  Synthetic linker sequence
GGGSGGG

SEQ ID NO: 165 - protein, 11 aa, Artificial Sequence
  Synthetic linker sequence
GGGSGGGSGGS

SEQ ID NO: 166 - protein, 9 aa, Artificial Sequence
  Synthetic sequence DDX3Y 428-136
FLLDILGAT

SEQ ID NO: 167 - protein, 9 aa, Artificial Sequence
  Synthetic sequence UTY 148-156
KAFQDVLYV

SEQ ID NO: 168 - protein, 9 aa, Artificial Sequence
  Synthetic sequence R1A alanine-substituted peptide
ALARLALVL

SEQ ID NO: 169 - protein, 9 aa, Artificial Sequence
  Synthetic sequence R4A alanine-substituted peptide
RLAALALVL

SEQ ID NO: 170 - protein, 9 aa, Artificial Sequence
  Synthetic sequence L5A alanine-substituted peptide
RLARAALVL

SEQ ID NO: 171 - protein, 9 aa, Artificial Sequence
  Synthetic sequence L7A alanine-substituted peptide
RLARLAAVL

SEQ ID NO: 172 - protein, 9 aa, Artificial Sequence
  synthetic sequence V8A alanine-substituted peptide
RLARLALAL

SEQ ID NO: 173 - protein, 12 aa, Artificial Sequence
  synthetic sequence TCR CDR3 alpha consensus
  VARIANT 2: Xaa = A, V, L, I, G, S, or T
  VARIANT 3: Xaa = A, V, G, L, I, S, T, P, or Y
  VARIANT 7: Xaa = A, V, G, L, I, or S
  VARIANT 8: Xaa = D or E
  VARIANT 10: Xaa = L, I, V, or A
  VARIANT (11)...(11): Xaa = T or S
CXXGGGXXGXXF

SEQ ID NO: 174 - protein, 13 aa, Artificial Sequence
  synthetic sequence TCR CDR3 beta consensus
  VARIANT 2: Xaa =  A, V, I, or L
  VARIANT 3: Xaa = S or T
  VARIANT 4: Xaa = S, T, or M
  VARIANT 5: Xaa = F, D, E, Y, Q, or P
  VARIANT 6: Xaa = L, I, V, A, M, F, or Q
  VARIANT (7)...(7): Xaa = s from 0 to 4 amino acids and optionally consists of G; S; SN; or PAGG
  VARIANT (8)...(8): Xaa = T, S, G, A, L, I, V, D, or E
  VARIANT (9)...(9): Xaa = N, D, Y, F, P, or G
  VARIANT (10)...(10): Xaa = E, T, or K
  VARIANT (11)...(11): Xaa = Q, N, A, L, I, or V
  VARIANT (12)...(12): Xaa = F or Y
CXXXXXXXXXXXF

(19) United States
(12) Patent Application Publication
(10) Pub. No.: US 2022/0267403 A1
(43) Pub. Date: Aug. 25, 2022

(54) BINDING PROTEINS SPECIFIC FOR 5T4 AND USES THEREOF

(71) Applicant: FRED HUTCHINSON CANCER RESEARCH CENTER, Seattle, WA (US)

(72) Inventors: Scott S. TYKODI, Bellevue, WA (US); Yuexin XU, Mercer Island, WA (US); Edus H. WARREN, Bainbridge Island, WA (US)

(21) Appl. No.: 16/768,617

(22) PCT Filed: Nov. 30, 2018
(86) PCT No.: PCT/US2018/063491

§ 371 Date: May 29, 2020

Related U.S. Application Data
(60) Provisional application No. 62/593,463, filed on Dec. 1, 2017.

(51) Int. Cl. C07K 14/725; A61P 35/04; A61K 35/17

(52) U.S. Cl. CPC C07K 14/7051; A61P 35/04; A61K 2039/5158; C07K 2319/03; A61K 35/17

(57) ABSTRACT

The present disclosure provides compositions and methods for targeting various tumor associated antigens (including human 5T4 epitopes), cells expressing high affinity antigen specific binding proteins such as TCRs, nucleic acids encoding the same, and compositions for use in treating diseases or disorders in which cells overexpress one or more of these antigens, such as in cancer.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 360056_449WO_SEQUENCE_LISTING.txt. The text file is 243 KB, was created on Nov. 30, 3018, and is being submitted electronically via EFS-Web.

### BACKGROUND

Adoptive transfer of tumor-specific T-cells is an appealing strategy to eliminate existing tumors and requires the establishment of a robust population of antigen-specific T cells in vivo to eliminate existing tumor and prevent recurrences (Stromnes et al., Immunol. Rev. 257:145, 2014). Although transfer of tumor-specific CD8⁺ cytotoxic T lymphocytes (CTLs) is safe and can mediate direct anti-tumor activity in select patients (Chapuis et al., Cancer Res. 72:LB-136, 2012; Chapuis et al., Sci. Transl. Med. 5:174ra127, 2013; Chapuis et al., Proc. Nat'l. Acad. Sci. U.S.A. 109:4592, 2012),²⁻⁴ the variability in the avidity of the CTLs isolated from each patient or donor limits the anti-tumor efficacy in clinical trials (Chapuis et al., 2013). Since TCR affinity is an important determinant of CTL avidity (Zoete et al., Frontiers Immunol. 4:268, 2013), strategies have been developed to redirect the antigen specificity of donor or patient T cells using high-affinity TCRα/β genes isolated from a well-characterized T cell clone specific for a tumor-specific antigen (Stromnes et al., Immunol. Rev. 257:145, 2014; Robbins et al., J. Clin. Oncol. 29:917, 2011). Such high-affinity self/tumor-reactive T cells are rare since T cells that express self/tumor-reactive TCRs are subject to central and peripheral tolerance (Stone and Kranz, Frontiers Immunol. 4:244, 2013), with relative TCR affinities varying widely between donors. Therefore, many matched donors must be screened to identify a sufficiently high-affinity tumor-specific T cell clone from which a TCRα/β gene therapy construct can be generated.

There is a clear need for alternative antigen-specific TCR immunotherapies directed against various cancers, such as renal, gastric, breast, colorectal, and ovarian cancers. Presently disclosed embodiments address these needs and provide other related advantages.

### BRIEF DESCRIPTION OF THE DRAWINGS

FIGS. 1A-1H show features of exemplary 5T4-specific binding proteins of the present disclosure. (A, B) ImMunoGeneTics (IMGT) sequence junction analysis of TRA (A) and TRB (B) CDR3 regions. Non-lettered boxes indicate N nucleotides and/or D regions (TRB) between V gene- and J gene-encoded sequence. At the right of FIGS. 1A and 1B is a key showing the IMGT amino acid classes. (C-F) Sequence logos showing conservation of TRA-CDR3 amino acids (two alignments; C, D) and TRB-CDR3 amino acids (two alignments; E, F) in a set of seven 5T4₁₇₋₂₅-specific TCRs of the present disclosure. (G) V and J gene usage for TRA and TRB by the seven TCRs are shown. (H) Net charges (R “Peptides” program) for TCRα and TCRβ CDR1, 2 and 3 amino acid sequences from 5T4₁₇₋₂₅-specific TCRs of the present disclosure, as calculated.

FIGS. 2A-2G relate to exemplary 5T4-specific binding protein lentivirus expression constructs of the present disclosure. (A) Schematic representation of TCR β chain (left) and TCR α chain (right), wherein the each chain comprises a variable β domain (V_{β}; encoded by a TRBV gene segment, a D gene segment, and a TRBJ gene segment) or a variable a domain (V_{α}; encoded by a TRAV gene and a TRAJ gene), respectively, and a TCR constant domain (encoded by a “C” gene segment). The indicated TRB, TRA and D alleles are for illustrating the expression construct, and do not necessarily correspond to gene usage of the presently disclosed exemplary 5T4-specific TCRs. (B) Schematic of a lentiviral expression construct that encodes a TCR as shown in (A). (C) Another schematic illustration of a 5T4-specific-TCR-coding construct, wherein the TCR constant domains from the β and α chains were mutated to contain cysteines at complementary positions to improve chain pairing. (D, E) flow cytometry data showing heterologous expression of 5T4₁₇₋₂₅ TCRs (codon-optimized) by lentiviral-transduced donor CD8⁺ T cells. Expression levels of seven 5T4₁₇₋₂₅ TCRs from four donors were determined at 7 days post-transduction (D) and 12 days post-sorting and expansion (E). Solid line with white=negative control; grey=5T4 TCR-transduced CD8⁺ T cells; dashed line with white=5T4 native T cell clone. (F) (upper panels) Heterologous cell surface expression of 5T4₁₇₋₂₅ specific TCRs (codon-optimized) stained by peptide:HLA (5T4₁₇₋₂₅:HLA-A*0201) tetramer at Day 7 post-transduction in comparison to tetramer staining of the native 5T4₁₇₋₂₅ specific T cell clone expressing the corresponding TCR. (Lower panels) Cell surface expression of 5T4₁₇₋₂₅-specific TCRs on healthy donor T cells is shown after sorting for 5T4₁₇₋₂₅: HLA-A*0201 tetramer-positive cells and following 12 days expansion. (G) 5T4₁₇₋₂₅-specific TCR expression on CD8⁺ T cells from healthy donors. The percentage of transduced donor CD8⁺ T cells expressing 5T4₁₇₋₂₅-specific TCRs stained by 5T4₁₇₋₂₅: HLA-A*0201 tetramer at day 7 post-transduction is shown for three different healthy donors.

FIGS. 3A and 3B provide flow cytometry data showing the knockdown of endogenous TCR expression in CD8⁺ T cells by transduction with a TRAC-targeting Crispr/Cas9-encoding lentivirus (A) and heterologous expression of a 5T4₁₇₋₂₅-specific TCR (B) in transduced, tetramer-sorted and expanded CD8⁺CD3⁻TCR⁻ T cells.

FIGS. 4A-4D show data from cytotoxicity assays in which CD8⁺ T cells expressing lentivirus-transduced TCR specific for 5T4₁₇₋₂₅ were co-cultured with 5T4⁺ tumor cell lines as follows: T2 cells with (black bars) or without (white bars) addition of 10 nM peptide antigen (A); lymphoblastoid (LCL; white bars) and renal cell carcinoma (RCC; black bars) target cell lines (B); breast carcinoma cell lines (C); colorectal tumor cells (D). All target cells were HLA-A*0201-positive except for BT-20 (4C).

FIGS. 5A-5G show specific killing activity and cytokine production by effector CD8⁺ T cells transduced to heterologously express 5T4-specific TCRs of the present disclosure. (A-C) Specific lysis (⁵¹Cr release; A) and interferon-release (IFN-γ in supernatant; B, C) data showing that CD8⁺ T cells transduced with 5T4-specific TCRs of the present disclosure recognize T2 target cells pulsed with the 5T4 peptide antigen. IFN-γ release was measured by ELISA in culture supernatants harvested after 18 hours co-culture of transduced CD8⁺ T cells with target antigen-pulsed T2 cells at 10:1 E:T. (D) Specific lysis by effector T cells of T2 cells pulsed with 10 nM of: 5T4₁₇₋₂₅ or control HLA-A2-binding peptides DDX3Y₄₂₈₋₄₃₆ or UTY₁₄₈₋₁₅₆ in a 4-hour cytotoxicity assay. The effector:target ratio (E:T) was 10:1. (E) Recognition by effector T cells of target antigen-pulsed T2 cells in a 4-hour cytotoxicity assay at 10:1 E:T. (F) TNF-α release (ELISA) in culture supernatants harvested following 18 hours co-culture of effector T cells with T2 cells pulsed with 10 nM of 5T4₁₇₋₂₅ or with the control peptide DDX3Y₄₂₈₋₄₃₆ (10:1 E:T). (G) TNF-α release (ELISA) in culture supernatants harvested after 18 hours co-culture of effector T cells with target antigen-pulsed T2 cells at 10:1 E:T.

FIG. 6A shows binding of alanine-substituted variant 5T4₁₇₋₂₅ peptides to HLA-A*0201 as determined by stabilization of HLA-A*0201 on the surface of peptide-pulsed T2 cells. The key at right shows the identifier and sequence of each alanine-substituted peptide. CMVpp65 is an HLA-A*0201⁺ T cell epitope from CMV and served as a positive control. Cell surface HLA-A*0201 was assessed by immunostaining and flow cytometry. FIG. 6B shows recognition by TCR-transduced CD8⁺ T cells of T2 cells pulsed with the progenitor 5T4₁₇₋₂₅ peptide or with HLA-A*0201-binding alanine-substituted variants (“R1A”; “R4A”) in a 4-hour cytotoxicity assay with a 10:1 E:T.

FIGS. 7A and 7B show cytotoxicity of 5T4₁₇₋₂₅-specific TCR-transduced CD8⁺ T cells against 5T4-expressing tumor target cells in a 4-hour cytotoxicity assay with 10:1 E:T. For each target cell-line, 5T4₁₇₋₂₅-specific TCR-transduced effector CD8⁺ T cells were plotted in the following order (L-R): clone2-6B9; clone15-3F10; clone19-5C2; clone21-7A10; clone3-6C3; clone17-9B5; clone6-5G8; and untransduced. In FIG. 7A, targets were: 5T4⁺/HLA-A*0201⁺ RCC cell lines (A498; BB65; TREP; DOBSKI); a 5T4⁺/HLA-A*0201⁻ RCC line (SST548) and the 5T47I-ILA-A*0201⁺ LCL (BB65-LCL). In FIG. 7B, targets were: a 5T4⁺/HLA-A*0201⁺ breast cancer line (MDA231); a 5T4⁺/HLA-A*0201⁻ breast cancer line (BT20s); and 5T4⁺/HLA-A*0201⁺ colon cancer line (SW480).

FIGS. 8A-8D relate to experiments examining the cytotoxicity of 5T4₁₇₋₂₅-specific TCR-transduced CD8⁺ T cells against target cells infected with a modified vaccinia Ankara virus that expresses 5T4 (“MVA-5T4”). FIG. 8A shows a schematic of human 5T4 protein; p17-25 is located in the signal sequence region. Trans: transmembrane region, Cyto: cytoplasmic region. FIG. 8B shows cell surface expression of 5T4 (immunostaining and flow) in T2 cells and BB65-LCL cells infected with MVA-WT or MVA-5T4 24 hours after infection. The fraction of cells positive for cell surface 5T4 after MVA-5T4 infection is indicated. CD8⁺ T cells expressing 5T4₁₇₋₂₅-specific TCRs were tested for recognition of T2 (C) and BB65-LCL cells (D) infected by MVA-WT or MVA-5T4, or of uninfected cells pulsed with 10 nM 5T4₁₇₋₂₅ peptide in a 6-hour cytotoxicity assay with 10:1 E:T.

### DETAILED DESCRIPTION

The present disclosure provides compositions and methods for targeting tumor-associated antigen 5T4, including, for example, cells expressing high affinity 5T4 antigen-specific binding proteins (e.g., TCRs), nucleic acids encoding the same, and compositions for use in treating diseases or disorders associated with aberrant expression (e.g., overexpression, improper expression) of 5T4 antigen (e.g., cancers). In certain embodiments, there are provided T cell receptors (TCRs) having high affinity for 5T4 peptide antigens associated with a major histocompatibility complex (MHC) (e.g., human leukocyte antigen, HLA) for use in, for example, adoptive immunotherapy to treat cancer.

By way of background, most tumor targets for T cell-based immunotherapies are self-antigens, since tumors arise from previously normal tissue. For example, such tumor-associated antigens (TAAs) may be expressed at high levels by a cancer cell, but may not be expressed or may be minimally expressed in other (e.g., healthy) cells. Oncofetal antigens, such as the trophoblast glycoprotein 5T4 (also called 5T4AG, 5T4 oncofetal antigen, M6P1, trophoblast glycoprotein, TBPG, Wnt-Activated Inhibitory Factor 1, and WAIF1), are generally present in healthy cells only during fetal development, but are expressed by certain cancerous cells in adults.

During T cell development in the thymus, T cells that bind weakly to self-antigens are allowed to survive in the thymus, and can undergo further development and maturation, while T cells that bind strongly to self-antigens are eliminated by the immune system since such cells may mount an undesirable autoimmune response. Hence, T cells are believed to be sorted by their relative ability to bind to antigens to prepare the immune system to respond against a foreign invader (i.e., recognition of non-self-antigen) while at the same time preventing an autoimmune response (i.e., recognition of self-antigen). Without wishing to be bound by theory, this tolerance mechanism can limit naturally occurring T cells that can recognize tumor (self) antigens with high affinity and, therefore, may eliminate T cells that can effectively eliminate tumor cells. Consequently, isolating T cells having high-affinity TCRs specific for tumor antigens is difficult because most such cells are essentially eliminated by the immune system.

An advantage of the instant disclosure is to provide binding proteins (e.g., TCRs and CARs) specific for 5T4 peptides, wherein a modified immune cell expressing such a binding protein is capable of binding to a 5T4 peptide; e.g., a 5T4 peptide:HLA complex expressed on a cell surface.

The compositions and methods described herein will in certain embodiments have therapeutic utility for the treatment of diseases and conditions associated with 5T4 expression. Such diseases include various forms of hyperproliferative disorders, including kidney cancer (e.g., renal cell carcinoma), colorectal cancer, lung cancer, prostate cancer, bladder cancer, cervical cancer, gastric cancer, non-small cell lung cancer (NSCLC), mesothelioma, ovarian cancer, pancreatic cancer, prostate, and breast cancer. Non-limiting examples of these and related uses are described herein and include in vitro, ex vivo and in vivo stimulation of 5T4 antigen-specific T cell responses, such as by the use of modified T cells comprising a heterologous polynucleotide that encodes a high-affinity or enhanced-affinity TCR specific for a 5T4 peptide (e.g., specific for a 5T4 peptide:HLA complex), or a heterologous polynucleotide that encodes a chimeric antigen receptor (CAR) specific for a 5T4 peptide.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain, hinge region, linker module) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, a “hematopoietic progenitor cell” is a cell that can be derived from hematopoietic stem cells or fetal tissue and is capable of further differentiation into mature cells types (e.g., immune system cells). Exemplary hematopoietic progenitor cells include those with a CD24^{Lo} Lin⁻ CD117⁺ phenotype or those found in the thymus (referred to as progenitor thymocytes).

As used herein, an “immune system cell” means any cell of the immune system that originates from a hematopoietic stem cell in the bone marrow, which gives rise to two major lineages: a myeloid progenitor cell (which give rise to myeloid cells such as monocytes, macrophages, dendritic cells, megakaryocytes and granulocytes); and a lymphoid progenitor cell (which give rise to lymphoid cells such as T cells, B cells and natural killer (NK) cells). Exemplary immune system cells include a CD4⁺ T cell, a CD8⁺ T cell, a CD4⁻ CD8⁻ double negative T cell, a γδ T cell, a regulatory T cell, a natural killer cell, and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

“Major histocompatibility complex” (MHC) refers to glycoproteins that deliver peptide antigens to a cell surface. MHC class I molecules are heterodimers having a membrane spanning a chain (with three a domains) and a non-covalently associated β2 microglobulin. MHC class II molecules are composed of two transmembrane glycoproteins, α and β, both of which span the membrane. Each chain has two domains. MHC class I molecules deliver peptides originating in the cytosol to the cell surface, where a peptide:MHC complex is recognized by CD8⁺ T cells. MHC class II molecules deliver peptides originating in the vesicular system to the cell surface, where they are recognized by CD4⁺ T cells. Human MHC is referred to as human leukocyte antigen (HLA).

A “T cell” is an immune system cell that matures in the thymus and produces T cell receptors (TCRs). T cells can be naïve (not exposed to antigen; increased expression of CD62L, CCR7, CD28, CD3, CD127, and CD45RA, and decreased expression of CD45RO as compared to T_{CM}), memory T cells (T_{M}) (antigen-experienced and long-lived), and effector cells (antigen-experienced, cytotoxic). T_{M} can be further divided into subsets of central memory T cells (T_{CM}, increased expression of CD62L, CCR7, CD28, CD127, CD45RO, and CD95, and decreased expression of CD54RA as compared to naïve T cells) and effector memory T cells (TEM, decreased expression of CD62L, CCR7, CD28, CD45RA, and increased expression of CD127 as compared to naïve T cells or T_{CM}). Effector T cells (T_{E}) refers to antigen-experienced CD8+ cytotoxic T lymphocytes that have decreased expression of CD62L, CCR7, CD28, and are positive for granzyme and perforin as compared to T_{CM}. Other exemplary T cells include regulatory T cells, such as CD4⁺ CD25⁺ (Foxp3⁺) regulatory T cells and Treg17 cells, as well as Tr1, Th3, CD8⁺CD28⁻, and Qa-1 restricted T cells.

“T cell receptor” or “TCR” refers to an immunoglobulin superfamily member (having a variable binding domain, a constant domain, a transmembrane region, and a short cytoplasmic tail; see, e.g., Janeway et al., Immunobiology: The Immune System in Health and Disease, 3^{rd} Ed Current Biology Publications, p. 4:33, 1997) that is capable of specifically binding to an antigen peptide bound to a MHC receptor. A TCR can be found on the surface of a cell or in soluble form and generally is comprised of a heterodimer having α and β chains (also known as TCRα and TCRβ, respectively), or γ and δ chains (also known as TCRγ and TCRδ, respectively). Like immunoglobulins (e.g., antibodies), the extracellular portion of TCR chains (e.g., α-chain, β-chain) contain two immunoglobulin domains: a variable domain (e.g., α-chain variable domain or V_{α}, β-chain variable domain or V_{β}; typically amino acids 1 to 116 based on Kabat numbering (Kabat et al., “Sequences of Proteins of Immunological Interest, US Dept. Health and Human Services, Public Health Service National Institutes of Health, 1991, 5^{th} ed.) at the N-terminal end, and one constant domain (e.g., α-chain constant domain or C_{α}, typically amino acids 117 to 259 based on Kabat, β-chain constant domain or C_{β}, typically amino acids 117 to 295 based on Kabat) at the C-terminal end and adjacent to the cell membrane. Also like immunoglobulins, the variable domains contain complementary determining regions (“CDRs”, also called hypervariable regions or “HVRs”) separated by framework regions (“FRs”) (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In certain embodiments, a TCR is found on the surface of a T cell (or T lymphocyte) and associates with the CD3 complex. The source of a TCR as used in the present disclosure may be from various animal species, such as a human, mouse, rat, rabbit or other mammal.

“CD3” is known in the art as a multi-protein complex of six chains (see, Abbas and Lichtman, 2003; Janeway et al., p. 172 and 178, 1999). In mammals, the complex comprises a CD3γ chain, a CD3δ chain, two CD3ε chains, and a homodimer of CD3ζ chains. The CD3γ, CD3δ, and CD3ε chains are related cell surface proteins of the immunoglobulin superfamily containing a single immunoglobulin domain. The transmembrane regions of the CD3γ, CD3δ, and CD3ε chains are negatively charged, which is a characteristic that is believed to allow these chains to associate with positively charged regions or residues of T cell receptor chains. The intracellular tails of the CD3γ, CD3δ, and CD3ε chains each contain a single conserved motif known as an immunoreceptor tyrosine-based activation motif or ITAM, whereas each CD3ζ chain has three ITAMs. Without wishing to be bound by theory, it is believed that the ITAMs are important for the signaling capacity of a TCR complex. CD3 as used in the present disclosure may be from various animal species, including human, mouse, rat, or other mammals.

As used herein, “TCR complex” refers to a complex formed by the association of CD3 with TCR. For example, a TCR complex can be composed of a CD3γ chain, a CD3δ chain, two CD3ε chains, a homodimer of CD3ζ chains, a TCRα chain, and a TCRβ chain. Alternatively, a TCR complex can be composed of a CD3γ chain, a CD3δ chain, two CD3ε chains, a homodimer of CD3ζ chains, a TCRγ chain, and a TCR chain.

A “component of a TCR complex,” as used herein, refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRP, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

“Chimeric antigen receptor” or “CAR” refers to a fusion protein that is engineered to contain two or more amino acid sequences linked together in a way that does not occur naturally or does not occur naturally in a host cell, which fusion protein can function as a receptor when present on a surface of a cell. CARs of the present disclosure include an extracellular portion comprising an antigen-binding domain (i.e., obtained or derived from an immunoglobulin or immunoglobulin-like molecule, such as a TCR specific for a cancer antigen, an scFv derived from an antibody, or an antigen binding domain derived or obtained from a killer immunoreceptor from an NK cell) linked to a transmembrane domain and one or more intracellular signaling domains (such as an effector domain, optionally containing co-stimulatory domain(s)) (see, e.g., Sadelain et al., Cancer Discov. 3:388 (2013); see also Harris and Kranz, Trends Pharmacol. Sci. 37:220 (2016), and Stone et al., Cancer Immunol. Immunother. 63:1163 (2014)).

A “linker” refers to an amino acid sequence that connects two proteins, polypeptides, peptides, domains, regions, or motifs and may provide a spacer function compatible with interaction of the two sub-binding domains so that the resulting polypeptide retains a specific binding affinity (e.g., scTCR) to a target molecule or retains signaling activity (e.g., TCR complex). In certain embodiments, a linker is comprised of about two to about 35 amino acids, for instance, or about four to about 20 amino acids or about eight to about 15 amino acids or about 15 to about 25 amino acids.

“Junction amino acids” or “junction amino acid residues” refer to one or more (e.g., about 2-10) amino acid residues between two adjacent motifs, regions or domains of a polypeptide, such as between a binding domain and an adjacent constant domain or between a TCR chain and an adjacent self-cleaving peptide. Junction amino acids may result from the construct design of a fusion protein (e.g., amino acid residues resulting from the use of a restriction enzyme site during the construction of a nucleic acid molecule encoding a fusion protein).

A “binding domain” (also referred to as a “binding region” or “binding moiety”), as used herein, refers to a molecule or portion thereof (e.g., peptide, oligopeptide, polypeptide, protein) that possesses the ability to specifically and non-covalently associate, unite, or combine with a target (e.g., 5T4, 5T4 peptide:MHC complex). A binding domain includes any naturally occurring, synthetic, semi-synthetic, or recombinantly produced binding partner for a biological molecule, a molecular complex (i.e., complex comprising two or more biological molecules), or other target of interest. Exemplary binding domains include TCR variable domains, single chain immunoglobulin variable regions (e.g., scTCR, scFv), receptor ectodomains, ligands (e.g., cytokines, chemokines), or synthetic polypeptides selected for their specific ability to bind to a biological molecule, a molecular complex or other target of interest. A binding domain of this disclosure may be contained in a binding protein, such as, for example, an immunoglobulin superfamily protein (e.g., TCR) or fusion protein that specifically binds to a 5T4 peptide antigen (e.g., CAR), including in complex with an HLA molecule.

As used herein, “specifically binds” or “specific for” refers to an association or union of a binding protein (e.g., TCR or CAR) or a binding domain (or fusion protein thereof) to a target molecule with an affinity or Kₐ (i.e., an equilibrium association constant of a particular binding interaction with units of 1/M) equal to or greater than 10⁵ M⁻¹ (which equals the ratio of the on-rate [kₒₙ] to the off-rate [k_{off}] for this association reaction), while not significantly associating or uniting with any other molecules or components in a sample. Binding proteins or binding domains (or fusion proteins thereof) may be classified as “high-affinity” binding proteins or binding domains (or fusion proteins thereof) or as “low-affinity” binding proteins or binding domains (or fusion proteins thereof). “High-affinity” binding proteins or binding domains refer to those binding proteins or binding domains having a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low-affinity” binding proteins or binding domains refer to those binding proteins or binding domains having a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M).

In certain embodiments, a receptor or binding domain may have “enhanced affinity,” which refers to selected or engineered receptors or binding domains with stronger binding to a target antigen than a wild type (or parent) binding domain. For example, enhanced affinity may be due to a Kₐ (equilibrium association constant) for the target antigen that is higher than the wild type binding domain, due to a K_{d} (dissociation constant) for the target antigen that is less than that of the wild type binding domain, due to an off-rate (k_{off}) for the target antigen that is less than that of the wild type binding domain, or a combination thereof. In certain embodiments, enhanced affinity TCRs may be codon optimized to enhance expression in a particular host cell, such as T cells (Scholten et al., Clin. Immunol. 119:135, 2006).

A variety of assays are known for identifying binding domains of the present disclosure that specifically bind a particular target, as well as determining binding domain or fusion protein affinities, such as Western blot, ELISA, analytical ultracentrifugation, spectroscopy and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

“5T4 antigen” or “5T4 peptide antigen” refers to a naturally or synthetically produced portion of a 5T4 protein ranging in length from about 7 amino acids to about 50 amino acids or from about 8 amino acids to about 25 amino acids or from about 9 amino acids to about 15 amino acids. In certain embodiments, a 5T4 antigen has the amino acid sequence RLARLALVL (SEQ ID NO:64), which is in certain instances referred to herein as 5T4₁₇₋₂₅, 5T4p₁₇₋₂₅, 5T4p17, or 5T4p. A 5T4 antigen can, in some embodiments, form a complex with a MHC (e.g., HLA) molecule and such a complex can bind with a TCR (or scTCR) specific for a 5T4 peptide:MHC (e.g., HLA) complex. In certain embodiments, a 5T4 antigen comprising or consisting of the amino acid sequence set forth in SEQ ID NO:64 can form a complex with an HLA-A2 moleule, such as an HLA-A*0201 molecule. In certain embodiments, a presently disclosed binding protein is capable of binding to a variant of SEQ ID NO:64 having the amino acid sequence ALARLALVL (SEQ ID NO:168).

The term “5T4-specific binding protein” refers to a protein or polypeptide that specifically binds to 5T4 or an antigenic peptide or antigenic fragment thereof. In some embodiments, a protein or polypeptide binds to 5T4 or a peptide thereof, such as a 5T4 peptide (e.g., 5T4p17) in complex with an MHC or HLA molecule, e.g., on a cell surface, with or with at least about a particular affinity. In certain embodiments, a 5T4-specific binding protein binds a 5T4-derived peptide:HLA complex (or 5T4-derived peptide:MHC complex) with a K_{d} of less than about 10⁻⁸M, less than about 10⁻⁹M, less than about 10⁻¹⁰ M, less than about 10⁻¹¹M, less than about 10⁻¹²M, or less than about 10⁻¹³M, or with an affinity that is about the same as, at least about the same as, or is greater than at or about the affinity exhibited by an exemplary 5T4-specific binding protein provided herein, such as any of the 5T4-specific TCRs provided herein, for example, as measured by the same assay. In certain embodiments, a 5T4-specific binding protein comprises a 5T4-specific immunoglobulin superfamily binding protein or binding portion thereof.

In any of the presently disclosed embodiments, a modified immune cell of the present disclosure (or a host cell expressing a fusion protein as disclosed herein) can bind via the 5T4-specific binding protein to a 5T4-derived peptide:HLA complex (e.g., a peptide HLA multimer, or a peptide:HLA complex expressed on the surface of a target cell) with high avidity. Assays and measures for determining avidity are known in the art and include those described herein; e.g., proliferation of modified immune or host cells, cytokine production by modified immune or host cells, and cytotoxic activity against target cells.

The term “5T4-binding domain” or “5T4-binding fragment” refers to a domain or portion of a 5T4-specific binding protein responsible for the specific 5T4 binding. A 5T4-specific binding domain alone (i.e., without any other portion of a 5T4-specific binding protein) can be soluble and can bind to 5T4 with a K_{d} of less than about 10⁻⁸M, less than about 10⁻⁹M, less than about 10⁻¹⁰ M, less than about 10⁻¹¹ M, less than about 10⁻¹²M, or less than about 10⁻¹³ M. Exemplary 5T4-specific binding domains include 5T4-specific scTCR (e.g., single chain αβTCR proteins such as Vα-L-Vβ, Vβ-L-Vα, Vα-Cα-L-Vα, or Vα-L-Vβ-Cβ, wherein Vα and Vβ are TCRα and β variable domains respectively, Cα and Cβ are TCRα and β constant domains, respectively, and L is a linker) and scFv fragments as described herein, which can be derived from an anti-5T4 TCR or antibody.

Principles of antigen processing by antigen presenting cells (APC) (such as dendritic cells, macrophages, lymphocytes or other cell types), and of antigen presentation by APC to T cells, including major histocompatibility complex (MHC)-restricted presentation between immunocompatible (e.g., sharing at least one allelic form of an MEW gene that is relevant for antigen presentation) APC and T cells, are known (see, e.g., Murphy, Janeway's Immunobiology (8^{th} Ed.) 2011 Garland Science, NY; chapters 6, 9 and 16). For example, processed antigen peptides originating in the cytosol (e.g., tumor antigen, intracellular pathogen) are generally from about 7 amino acids to about 11 amino acids in length and will associate with class I MEW molecules, whereas peptides processed in the vesicular system (e.g., bacterial, viral) will vary in length from about 10 amino acids to about 25 amino acids and associate with class II MEW molecules.

An “altered domain” or “altered protein” refers to a motif, region, domain, peptide, polypeptide, or protein with a non-identical sequence identity to a wild type motif, region, domain, peptide, polypeptide, or protein (e.g., a wild type TCRα chain, TCRβ chain, TCRα constant domain, TCRβ constant domain) of at least 85% (e.g., 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%).

As used herein, “nucleic acid” or “nucleic acid molecule” refers to any of deoxyribonucleic acid (DNA), ribonucleic acid (RNA), oligonucleotides, fragments generated, for example, by the polymerase chain reaction (PCR) or by in vitro translation, and fragments generated by any of ligation, scission, endonuclease action, or exonuclease action. In certain embodiments, the nucleic acids of the present disclosure are produced by PCR. Nucleic acids may be composed of monomers that are naturally occurring nucleotides (such as deoxyribonucleotides and ribonucleotides), analogs of naturally occurring nucleotides (e.g., α-enantiomeric forms of naturally-occurring nucleotides), or a combination of both. Modified nucleotides can have modifications in or replacement of sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. Nucleic acid molecules can be either single stranded or double stranded.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region (“leader and trailer”) as well as intervening sequences (introns) between individual coding segments (exons).

As used herein, the term “host” refers to a cell (e.g., T cell) or microorganism targeted for genetic modification with a heterologous nucleic acid molecule to produce a polypeptide of interest (e.g., high or enhanced affinity anti-5T4 TCR). In certain embodiments, a host cell may optionally already possess or be modified to include other genetic modifications that confer desired properties related or unrelated to, e.g.: biosynthesis of the heterologous protein (e.g., inclusion of a detectable marker; deleted, altered or truncated endogenous TCR; or increased co-stimulatory factor expression). In certain embodiments, a host cell is a human hematopoietic progenitor cell transduced with a heterologous nucleic acid molecule encoding a TCRα chain specific for a 5T4 antigen peptide.

As used herein, the term “recombinant” refers to a cell, microorganism, nucleic acid molecule, or vector that has been genetically engineered by human intervention—that is, modified by introduction of a heterologous nucleic acid molecule, or refers to a cell or microorganism that has been altered such that expression of an endogenous nucleic acid molecule or gene is controlled, deregulated or constitutive. For example, in certain embodiments, a “modified” immune cell is provided wherein the immune cell is modified (e.g., genetically engineered) to contain a heterologous polynucleotide that encodes a binding protein that specifically binds a 5T4₁₇₋₂₅ peptide. Human-generated genetic alterations may include, for example, modifications that introduce nucleic acid molecules (which may include an expression control element, such as a promoter) that encode one or more proteins or enzymes, or other nucleic acid molecule additions, deletions, substitutions, or other functional disruption of or addition to a cell's genetic material. It will be understood that a “modified” immune cell of the present disclosure refers to an engineered host immune cell (e.g., a host cell that is transduced to contain or express a heterologous polynucleotide), as well as to progeny of the engineered host immune cell that contain the same modification. Exemplary modifications include those in coding regions or functional fragments thereof of heterologous or homologous polypeptides from a reference or parent molecule.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” is recognized in the art as a substitution of one amino acid for another amino acid that has similar properties. Exemplary conservative substitutions are well known in the art (see, e.g., WO 97/09433 at page 10; Lehninger, Biochemistry, 2^{nd} Edition; Worth Publishers, Inc. NY, N.Y., pp. 71-′7′7, 1975; Lewin, Genes IV, Oxford University Press, NY and Cell Press, Cambridge, Mass., p. 8, 1990).

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Exemplary vectors are those capable of autonomous replication (episomal vector) or expression of nucleic acid molecules to which they are linked (expression vectors).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As used herein, “expression vector” refers to a DNA construct containing a nucleic acid molecule that is operably-linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus” and “vector” are often used interchangeably.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof.

The term “introduced” in the context of inserting a nucleic acid molecule into a cell, means “transfection”, or “transformation” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

As used herein, “heterologous” nucleic acid molecule, construct or sequence refers to a nucleic acid molecule or portion of a nucleic acid molecule that is not native to a host cell, but may be homologous to a nucleic acid molecule or portion of a nucleic acid molecule from the host cell. The source of the heterologous nucleic acid molecule, construct or sequence may be from a different genus or species. In certain embodiments, a heterologous nucleic acid molecule is added (i.e., not endogenous or native) to a host cell or host genome by, for example, conjugation, transformation, transfection, electroporation, or the like, wherein the added molecule may integrate into the host genome or exist as extra-chromosomal genetic material (e.g., as a plasmid or other form of self-replicating vector), and may be present in multiple copies. In addition, “heterologous” refers to a non-native enzyme, protein or other activity encoded by a heterologous polynucleotide introduced into the host cell, even if the host cell encodes a homologous protein or activity.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. For example, as disclosed herein, a host cell can be modified to express two or more heterologous nucleic acid molecules encoding desired TCR specific for a 5T4 antigen peptide (e.g., TCRα and TCRβ). When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

As used herein, the term “endogenous” or “native” refers to a gene, protein, or activity that is normally present in a host cell. Moreover, a gene, protein or activity that is mutated, overexpressed, shuffled, duplicated or otherwise altered as compared to a parent gene, protein or activity is still considered to be endogenous or native to that particular host cell. For example, an endogenous control sequence from a first gene (e.g., promoter, translational attenuation sequences) may be used to alter or regulate expression of a second native gene or nucleic acid molecule, wherein the expression or regulation of the second native gene or nucleic acid molecule differs from normal expression or regulation in a parent cell.

The term “homologous” or “homolog” refers to a molecule or activity found in or derived from a host cell, species or strain. For example, a heterologous polynucleotide may be homologous to a native host cell gene, and may optionally have an altered expression level, a different sequence, an altered activity, or any combination thereof.

“Sequence identity” or “percent identity,” as used herein, in the context of two or more polypeptide or polynucleotide sequences, means two or more sequences or subsequences that are the same or have a specified percentage of amino acid residues or nucleotides that are the same over a specified region (e.g., 60%, 65%, 70%, 75%, 80%, 85%, 90%, 91%, 92%, 93%, 94%, 95%, 96%, 97%, 98%, 99%, or 100% identity), when compared and aligned for maximum correspondence over a comparison window, or designated region, as measured using a sequence comparison algorithm, by manual alignment, or by visual inspection. A preferred algorithm suitable for determining percent sequence identity and sequence similarity is LALIGN, available at, for example, www.ebi.ac.uk/Tools/psa/lalign/nucleotide.html for polynucleotides or www.ebi.ac.uk/Tools/psa/lalign for polypeptides (see Huang and Miller, Adv. Appl. Math. 12:337-357, 1991).

As used herein, “hyperproliferative disorder” refers to excessive growth or proliferation as compared to a normal or undiseased cell. Exemplary hyperproliferative disorders include tumors, cancers, neoplastic tissue, carcinoma, sarcoma, malignant cells, pre-malignant cells, as well as non-neoplastic or non-malignant hyperproliferative disorders (e.g., adenoma, fibroma, lipoma, leiomyoma, hemangioma, fibrosis, restenosis, as well as autoimmune diseases such as rheumatoid arthritis, osteoarthritis, psoriasis, inflammatory bowel disease, or the like).

### Binding Proteins, Modified Immune Cells, and Fusion Proteins Specific for 5T4

In certain aspects, the present disclosure provides a modified immune cell comprising a heterologous polynucleotide that encodes a binding protein (e.g, a TCR, a single chain TCR (scTCR), or a CAR) that specifically binds to a 5T4 or a 5T4 peptide antigen.

5T4 (also known as 5T4AG, 5T4 oncofetal antigen, M6P1, trophoblast glycoprotein, TPBG, Wnt-Activated Inhibitory Factor 1, and WAIF1) is an N-glycosylated transmembrane glycoprotein that is expressed in fetal trophoblasts and in malignant tumors in adults (e.g., kidney, colorectal, ovarian, bladder, breast, cervical, lung, pancreatic, prostate, and gastric; see Stern and Harrop, Cancer Immunol. Immunother. 66:415, 2016), but has very limited expression in normal adult tissues (Starzynska et al. Br. J. Cancer 66(5):867-79 (1992); see also Southall et al., Br. J. Cancer (61):89-95 (1990)). By way of background, 5T4 was identified as a shared surface molecule between human trophoblasts and cancer cells on the theory that such molecules may allow survival of the fetus as a semi-allograft in the mother, and, analogously, of a tumor as a semi-allograft in its adult host. Increased expression of 5T4 is associated with disease development and progression and poor clinical outcomes. Stern et al., Semin. Cancer Biol. (29):13-20 (2014).

Previous therapies targeting 5T4 have involved the use of a murine monoclonal antibody (mAb5T4) and single chain variable fragments (scFvs) derived therefrom (see Shaw et al., Biochim. Biophys. Acta 1542(2-3):238-246 (2000)), or an antibody-drug conjugate comprising a 5T4 antibody conjugated to the DNA-damaging agent calicheamicin (see Damelin et al., Cancer Res. 71(12):4236 (2011)). Another approach relates to a vaccine called TroVax® (being developed by Oxford Biomedica). Briefly, TroVax® is an attenuated, but still highly immunogenic, strain of modified vaccinia virus Ankara that encodes 5T4. Harrop et al. Cancer Immunol. Immunother. 55:1081 (2006); see also Tykodi and Thompson, Expert Opin. Biol. Ther. 8:1947 (2008). Expression of the 5T4 transgene using TroVax® induced a cellular immune response in colorectal cancer patients. (Rowe and Cen, Hum. Vaccin. Immunother. 10:3196 (2014)). In another clinical study, a so-called “superantigen” comprised of a murine antigen-binding fragment (Fab) recognizing 5T4 and a modified Staphylococcal enterotoxin A (anatumomab mafenatox; “ABR-214936”) was administered to renal cell carcinoma patients. Shaw et al., Br. J. Cancer 96:567-574 (2007).

Binding Proteins

In some embodiments, binding proteins (e.g., that are heterologously expressed by a modified host immune cell) of the present disclosure include CDR3 regions with negative net charges and are able to specifically associate with a 5T4 peptide:HLA complex, wherein the 5T4 peptide comprises or consists of the amino acid sequence RLARLALVL (SEQ ID NO:64), which carries a net positive charge. CDR sequences can be determined according to known methods, and net charges of amino acid sequences, including of presently disclosed CDRs, can be determined using, for example, the R package “Peptides” (Rice et al., Trends Genet. 16(6):276-277 (2000)).

In certain embodiments, a modified immune cell of the present disclosure comprises a heterologous polynucleotide that encodes a binding protein, wherein the encoded binding protein comprises: (a) a T cell receptor (TCR) α-chain variable (V_{α}) domain comprising a CDR3 amino acid sequence (CDR3α) having a net charge of about −0.01 to about −2.2 (i.e., including all values therebetween; e.g., including about −0.01, about −0.05, about −0.1, about −0.5, about −1.0, about −1.5, about −2.0, about −2.2, or the like). and a TCR β-chain variable (V_{β}) domain; or (b) a TCR V_{α} domain, and a TCR V_{β} domain comprising a CDR3 amino acid sequence (CDR3β) having a net charge of about −0.01 to about −2.2; or (c) a TCR V_{α} domain of (a), and a TCR V_{β} domain of (b), wherein the encoded binding protein is capable of specifically binding to a 5T4 peptide:HLA complex, wherein the 5T4 peptide comprises or consists of (i) the amino acid sequence RLARLALVL (SEQ ID NO:64) or (ii) the amino acid sequence ALARLALVL (SEQ ID NO:168).

In certain embodiments, (i) the CDR3α has a net charge of about −0.01 to about −2.2 and; (ii) the CDR3β has a net charge of about −0.01 to about −2.2.

In some embodiments, (i) the CDR3β has a net charge of about −0.05 to about −2.2; and (ii) the CDR3α has a net charge of about −0.05 to about −2.2.

In some embodiments, the CDR3α has a net charge of about −0.05 to about −2.2. In other embodiments, the CDR3α has a net charge of about −1.0 to about −2.2.

In some embodiments, the CDR3β has a net charge of about −0.05 to about −1.0. In other embodiments, the CDR3β has a net charge of about −1.0 to about −2.2.

In some embodiments, one of (i) the CDR3α or (ii) the CDR3β has a net charge of about −0.05; and the other of (i) the CDR3α or (ii) the CDR3β has a net charge of about −1.0. For example, in certain embodiments, the CDR3α has a net charge of about −0.05 and the CDR3β has a net charge of about −1.0. In other embodiments, the CDR3α has a net charge of about −1.0 and the CDR3β has a net charge of about −0.05.

In other embodiments, the CDR3α and the CDR3β each have a net charge of about −1.0. In further embodiments, the CDR3α and the CDR3β each have a net charge of about −1.05.

In still other embodiments, the CDR3α and the CDR3β each have a net charge of about −2.2. In further embodiments, the CDR3α and the CDR3β each have a net charge of about −2.05.

In any of the embodiments disclosed herein, the CDR3α and/or the CDR3β can have a net charge of an exemplary CDR3α or CDR3β as set forth in Table 1, or can have a net charge that is about the same as the net charge of an exemplary CDR3α or CDR3β (respectively) as set forth in Table 1. In some embodiments, an encoded binding protein of the present disclosure comprises a CDR3α and a CDR3β that have net charges that are about the same as the net charges of an exemplary clone as set forth in Table 1 (e.g., clone2-6B8; clone15-3F10; clone6-5G8; clone17-9B5; clone3-6C3; clone19-5C2; clone21-7A10).

TABLE 1
CDR3 Net charges of 5T4-specific TCRs.

| Clone Name | CDR3α net charge | CDR3β net charge |
|---|---|---|
| Clone2-6B8 | −2.063097448 | −2.060440171 |
| Clone15-3F10 | −1.064394347 | −1.062215294 |
| Clone6-5G8 | −1.063543932 | −1.064394347 |
| Clone17-9B5 | −1.063543932 | −1.063065708 |
| Clone3-6C3 | −1.063543932 | −0.06468938 |
| Clone19-5C2 | −0.064397658 | −1.063916122 |
| Clone21-7A10 | −1.063951174 | −2.06176881 |

In some embodiments, 5T4-specific binding proteins of the present disclosure (and including binding domains of disclosed fusion proteins) are encoded by common alleles or combinations of common alleles. For example, in certain embodiments, the V_{α} domain is encoded by a TRAV allele and a TRAJ allele and the V_{β} domain is encoded by a TRBV allele and a TRBJ allele, wherein: (i) the TRAV allele is αV1-1, αV12-2, αV38-1, or αV38-2; (ii) the TRAJ allele is αJ26, αJ39, αJ42, or αJ45; (iii) the TRBV allele is βV4-3, βV6-3, βV10-2, βV18, or βV10-2; (iv) the TRBJ allele is βJ1-1, βJ2-1, βJ2-2, βJ2-3, or βJ2-7; or (v) any combination of (i)-(iv).

In certain embodiments, the TRAV allele is αV38-2 and the TRAJ allele is αJ45.

In certain embodiments, the TRBV allele is βV6-3.

In certain embodiments, the TRBJ allele is βJ2-7.

In particular embodiments: (i) the TRAV allele is αV38-2; (ii) the TRAJ allele is αJ45; (iii) the TRBV allele is βV6-3; and (iv) the TRBJ allele is βJ2-7.

In certain embodiments, a 5T4-specific binding protein of the present disclosure comprises a CDR3α having the consensus sequence of CX₁X₂GGGX₃X₄GX₅X₆F (SEQ ID NO:173), wherein: X₁ is A, V, L, I, G, S, or T; X₂ is A, V, G, L, I, S, T, P, or Y; X₃ is A, V, G, L, I, or S; X₄ is D or E; X₅ is L, I, V, or A; and X₆ is T or S. In further embodiments, X₁ is A or S; X₂ is S or G; X₃ is A; X₄ is D; X₅ is L; and/or X₆ is T.

In certain embodiments, a 5T4-specific binding protein of the present disclosure comprises a CDR3β having the consensus sequence CX₇X₈X₉X₁₀X₁₁X₁₂X₁₃X₁₄X₁₅X₁₆X₁₇F (SEQ ID NO:174), wherein: X₇ is A, V, I, or L; X₈ is S or T; X₉ is S, T, or M; X₁₀ is F, D, E, Y, Q, or P; X₁₁ is L, I, V, A, M, F, or Q; X₁₂ is from 0 to 4 amino acids and optionally consists of: G, S, SN, or PAGG; X₁₃ is T, S, G, A, L, I, V, D, or E; X₁₄ is N, D, Y, F, P, or G; X₁₅ is E, T, or K; X₁₆ is Q, N, A, L, I, or V; and X₁₇ is F or Y. In further embodiments, X₇ is A; X₈ is 5; X₉ is 5; X₁₅ is E; and/or X₁₆ is Q. In particular embodiments, a 5T4-specific binding protein of the present disclosure comprises (i) a CDR3α having the consensus sequence of CX₁X₂GGGX₃X₄GX₅X₆F (SEQ ID NO:173), wherein: X₁ is A or S; X₂ is S or G; X₃ is A; X₄ is D; X₅ is L; and/or X₆ is T; and (ii) a CDR3β having the consensus sequence CX₇X₈X₉X₁₀X₁₁X₁₂X₁₃X₁₄X₁₅X₁₆X₁₇F (SEQ ID NO:174) as disclosed herein, wherein: X₇ is A; X₈ is 5; X₉ is 5; X₁₅ is E; and/or X₁₆ is Q.

In certain embodiments, a binding protein (e.g., encoded by a heterologous polynucleotide contained in a modified immune cell) is provided, wherein the binding protein comprises: (a) a T cell receptor (TCR) α-chain variable (V_{α}) domain having a CDR3 amino acid sequence shown in any one of SEQ ID NOS:49-55, and a TCR β-chain variable (V_{β}) domain; or (b) a TCR V_{α} domain, and a TCR V_{β} domain having a CDR3 amino acid sequence shown in any one of SEQ ID NOS:56-62; or (c) a TCR V_{α} domain of (a), and a TCR V_{β} domain of (b).

In particular embodiments, a binding protein comprises a CDR3α having the amino acid sequence shown in SEQ ID NO:49 and a CDR3β having the amino acid sequence shown in SEQ ID NO:56. In other embodiments, a binding protein comprises a CDR3α having the amino acid sequence shown in SEQ ID NO:50 and a CDR3β having the amino acid sequence shown in SEQ ID NO:57. In other embodiments, a binding protein comprises a CDR3α having the amino acid sequence shown in SEQ ID NO:51 and a CDR3β having the amino acid sequence shown in SEQ ID NO:58. In other embodiments, a binding protein comprises a CDR3α having the amino acid sequence shown in SEQ ID NO:52 and a CDR3β having the amino acid sequence shown in SEQ ID NO:59. In other embodiments, a binding protein comprises a CDR3α having the amino acid sequence shown in SEQ ID NO:53 and a CDR3β having the amino acid sequence shown in SEQ ID NO:60. In other embodiments, a binding protein comprises a CDR3α having the amino acid sequence shown in SEQ ID NO:54 and a CDR3β having the amino acid sequence shown in SEQ ID NO:61. In other embodiments, a binding protein comprises a CDR3α having the amino acid sequence shown in SEQ ID NO:55 and a CDR3β having the amino acid sequence shown in SEQ ID NO:62.

In some embodiments, a binding protein (or a fusion protein of the instant disclosure) comprises a CDR3α having the amino acid sequence shown in any one of SEQ ID NOs:50, 51, and 54, and a CDR3β having the amino acid sequence shown in any one of SEQ ID NOs:57, 58, and 60.

In some aspects, the present disclosure provides a modified immune cell comprising a heterologous polynucleotide that encodes a binding protein, wherein the binding protein comprises: (a) a T cell receptor (TCR) α-chain variable (V_{α}) domain having a CDR3 amino acid sequence shown in any one of SEQ ID NOS:49-55, and a TCR β-chain variable (V_{β}) domain; or (b) a TCR V_{α} domain, and a TCR V_{β} domain having a CDR3 amino acid sequence shown in any one of SEQ ID NOS:56-62; or (c) a TCR V_{α} domain of (a), and a TCR V_{β} domain of (b), and wherein the encoded binding protein is capable of specifically binding to a 5T4 peptide. In further embodiments, the encoded binding protein is capable of specifically binding to a 5T4 peptide:HLA complex, such as a 5T4-specific TCR.

In any of the embodiments described herein, an encoded binding protein is capable of specifically binding to a RLARLALVL (SEQ ID NO:64):human leukocyte antigen (HLA) complex with a K_{d} less than or equal to about 10⁻⁸M.

In some embodiments, the encoded binding protein is capable of specifically binding to a 5T4 peptide:HLA complex on a cell surface independent of CD8 or in the absence of CD8.

In some embodiments, the encoded binding protein specifically binds to a RLARLALVL (SEQ ID NO:64):HLA-A*201 complex and can also bind to a ALARLALVL (SEQ ID NO:168):HLA-A*201 complex.

In any of the embodiments disclosed herein, the V_{α} domain and the V_{β} domain of a binding protein can each comprise a CDR1 amino acid sequence (CDR1α; CDR1β) and a CDR2 amino acid sequence (CDR2α; CDR2β), wherein: (i) the CDR1α has a net charge of about −2.0 to about 0 (e.g., about −2.0, about −1.5, about −1.0, about −0.5, or about 0); (ii) the CDR1β has a net charge of about −1.0 to about 1.5 (e.g., about −1.0, about −0.5, about 0, about 0.5, about 1.0. or about 1.5); (iii) the CDR2α has a net charge of about 0 to about −1.0 (e.g., about 0, about −0.5, or about −1.0); and/or (iv) the CDR2β has a net charge of about 0 to about −1.0.

In certain embodiments, the CDR1α has a net charge of about 0 to about −1.0. In other embodiments, the CDR1α has a net charge of about −1.0 to about −2.0.

In certain embodiments, the CDR1β has a net charge of about 0 to about 1.5. In further embodiments, the CDR1β has a net charge of about 0.01 to about 1.2. In some embodiments, the CDR1β has a net charge of about −1.0.

In certain embodiments, the CDR2α has a net charge of about 0 to about −1.0. In some embodiments, the CDR2α has a net charge of about 0. In some embodiments, the CDR2α has a net charge of about −1.0

In certain embodiments, the CDR2β has a net charge of about 0 to about −1.0. In some embodiments, the CDR2β has a net charge of about 0. In some embodiments, the CDR2β has a net charge of about −1.0.

In any of the embodiments disclosed herein, the CDR1α, CDR1β, CDR2α, and/or CDR2β, can comprise a net charge of an exemplary CDR1α, CDR1β, CDR2α, and/or CDR2β as set forth in Table 2, or can have a net charge that is about the same as the net charge of an exemplary CDR1α, CDR1β, CDR2α, and/or CDR2β (respectively) as set forth in Table 2. In some embodiments, an encoded binding protein of the present disclosure comprises a CDR1α, CDR1β, CDR2α, and a CDR2β that have net charges that are about the same as the net charges of an exemplary clone as set forth in Table 2 (e.g., clone2-6B8; clone15-3F10; clone6-5G8; clone17-9B5; clone3-6C3; clone19-5C2; clone21-7A10).

TABLE 2
CDR1 and CDR2 Net charges of 5T4-specific TCRs.

| | CDR1α | CDR1β | CDR2α | CDR2β |
|---|---|---|---|---|
| Clone Name | net charge | net charge | net charge | net charge |
| Clone2-6B8 | −0.001572528 | 0.088042976 | −1.002419631 | −1.001569216 |
| Clone15-3F10 | −1.001941406 | −0.910181901 | −0.001386026 | −1.000240578 |
| Clone6-5G8 | −2.001494922 | −0.910181901 | −0.001386026 | −1.000240578 |
| Clone17-9B5 | −2.001494922 | −0.910181901 | −0.001386026 | −1.000240578 |
| Clone3-6C3 | −2.001494922 | −0.910181901 | −0.001386026 | −1.000240578 |
| Clone19-5C2 | −2.001494922 | 0.08889339 | −0.001386026 | −0.999319181 |
| Clone21-7A10 | −0.002866115 | 1.179507447 | −1.001569216 | −0.000535612 |

In any of the embodiments disclosed herein, an encoded 5T4-specific binding protein can comprise CDRs (e.g., 1α, 1β, 2α, 2β, 3α, 3β) having net charges that are about the same as the net charges of CDRs of the exemplary clones set forth in Table 1 and Table 2.

In some embodiments, an encoded binding protein of the present disclosure (e.g., a CAR, a TCR, or an scTCR) specific for 5T4 may include an amino acid sequence that has at least 85%, 90%, 91%, 92%, 93%, 94%, 95%, 96%, 97%, 98%, or 99% amino acid sequence identity to any of the exemplary amino acid sequences disclosed herein (e.g., SEQ ID NOS:1-14), provided that (a) at least three or four of the CDRs have no mutations, (b) the CDRs that do have mutations have only up to two to four amino acid substitutions, insertions, deletions, or a combination thereof, and (c) the binding protein retains its ability to specifically bind to a 5T4 peptide antigen:HLA complex (e.g., on a cell surface, or as an peptide:HLA multimer in vitro, such as a tetramer). In further embodiments, a binding protein retains its ability to bind to a 5T4 peptide antigen:HLA complex about the same as a parent TCR from which the binding protein was derived, or with a K_{d} less than or equal to about 10⁻⁸M.

In certain embodiments, binding proteins are provided that include an amino acid sequence that has at least 85%, 90%, 91%, 92%, 93%, 94%, 95%, 96%, 97%, 98%, or 99% amino acid sequence identity to any of the exemplary amino acid sequences disclosed herein (e.g., SEQ ID NOS:1-14, or any combination thereof) and can specifically bind to a 5T4 peptide antigen:HLA complex (e.g., a 5T4p17:HLA-A*201 complex).

In any of the aforementioned embodiments, the present disclosure provides a modified immune cell comprising a heterologous polynucleotide that encodes a T cell receptor (TCR), comprising an α-chain and a β-chain, wherein the TCR binds to a 5T4:HLA-A*201 complex (e.g., a 5T4₁₇₋₂₅:HLA-A*0201 complex) on a cell surface independent of, or in the absence of, CD8.

In any of the aforementioned embodiments, the present disclosure provides a modified immune cell comprising a heterologous polynucleotide that encodes a 5T4-specific binding protein, wherein the encoded binding protein comprises a V_{α} domain comprising or consisting of an α-chain variable domain having the amino acid sequence of any one of SEQ ID NOS:1-7, a V_{β} domain comprises or consists of a β-chain variable domain having the amino acid sequence of any one of SEQ ID NOS:8-14, or any combination thereof.

In particular embodiments, the V_{α} domain comprises or consists of the amino acid sequence of SEQ ID NO:1 and the V_{β} domain comprises or consists of the amino acid sequence of SEQ ID NO:8.

In other embodiments, the V_{α} domain comprises or consists of the amino acid sequence of SEQ ID NO:2 and the V_{β} domain comprises or consists of the amino acid sequence of SEQ ID NO:9.

In other embodiments, the V_{α} domain comprises or consists of the amino acid sequence of SEQ ID NO:3 and the V_{β} domain comprises or consists of the amino acid sequence of SEQ ID NO:10.

In other embodiments, the V_{α} domain comprises or consists of the amino acid sequence of SEQ ID NO:4 and the V_{β} domain comprises or consists of the amino acid sequence of SEQ ID NO:11.

In other embodiments, the V_{α} domain comprises or consists of the amino acid sequence of SEQ ID NO:5 and the V_{β} domain comprises or consists of the amino acid sequence of SEQ ID NO:12.

In other embodiments, the V_{α} domain comprises or consists of the amino acid sequence of SEQ ID NO:6 and the V_{β} domain comprises or consists of the amino acid sequence of SEQ ID NO:13.

In other embodiments, the V_{α} domain comprises or consists of the amino acid sequence of SEQ ID NO:7 and the V_{β} domain comprises or consists of the amino acid sequence of SEQ ID NO:14.

In further embodiments, the encoded binding protein comprises a V_{α} domain comprising or consisting of the amino acid sequence shown in any one of SEQ ID NOs:2, 3, or 5, and a V_{β} domain comprising or consisting of the amino acid sequence shown in any one of SEQ ID NOs:9, 10, or 12.

In certain embodiments, the encoded binding protein further comprises an α-chain constant (C_{α}) domain having at least 90% sequence identity to the amino acid sequence shown in SEQ ID NO:15.

In some embodiments, the encoded binding protein further comprises a β-chain constant (C_{β}) domain having at least 90% sequence identity to the amino acid sequence shown in SEQ ID NO:16 or 17.

In some embodiments, the C_{α} domain and the C_{β} domain of the encoded binding protein each comprise a substituted (i.e., non-native) cysteine amino acid, wherein the substituted cysteine amino acids are in complementary positions (i.e., are spatially arranged in close proximity, such as, for example, proximity sufficient to form a disulfide bridge therebetween) in the C_{α} domain and the C_{β} domain when the C_{α} domain and the C_{β} domain associate to form a dimer (see, e.g., Cohen et al., Cancer Res. 67(8):3898-3903 (2007)). In some embodiments, a C_{α} modification comprises a substitution of a cysteine for a native threonine corresponding to position 47 of human TRAC (sequence as set forth in Uniprot P01848) and a C_{β} modification comprises a substitution of a cysteine for native serine corresponding to position 56 of human TRBC (sequence as set forth in Uniprot P01850); see also Kuball et al., Blood 109(6):2331-2338 (2007), referring to cysteine mutations at positions 48 (TRAC) and 57 (TRBC).

Accordingly, in particular embodiments, the encoded binding protein comprises a V_{α} domain comprising or consisting of SEQ ID NO:1, a V_{β} domain comprising or consisting of SEQ ID NO:8, a C_{α} domain comprising or consisting of SEQ ID NO:15, and a C_{β} domain comprising or consisting of SEQ ID NO:16 or SEQ ID NO:17.

In other embodiments, the encoded binding protein comprises a V_{α} domain comprising or consisting of SEQ ID NO:2, a V_{β} domain comprising or consisting of SEQ ID NO:9, a C_{α} domain comprising or consisting of SEQ ID NO:15, and a C_{β} domain comprising or consisting of SEQ ID NO:16 or SEQ ID NO:17.

In still other embodiments, the encoded binding protein comprises a V_{α} domain comprising or consisting of SEQ ID NO:3, a V_{β} domain comprising or consisting of SEQ ID NO:10, a C_{α} domain comprising or consisting of SEQ ID NO:15, and a C_{β} domain comprising or consisting of SEQ ID NO:16 or SEQ ID NO:17.

In yet other embodiments, the encoded binding protein comprises a V_{α} domain comprising or consisting of SEQ ID NO:4, a V_{β} domain comprising or consisting of SEQ ID NO:11, a C_{α} domain comprising or consisting of SEQ ID NO:15, and a C_{β} domain comprising or consisting of SEQ ID NO:16 or SEQ ID NO:17.

In still other embodiments, the encoded binding protein comprises a V_{α} domain comprising or consisting of SEQ ID NO:5, a V_{β} domain comprising or consisting of SEQ ID NO:12, a C_{α} domain comprising or consisting of SEQ ID NO:15, and a C_{β} domain comprising or consisting of SEQ ID NO:16 or SEQ ID NO:17.

In yet other embodiments, the encoded binding protein comprises a V_{α} domain comprising or consisting of SEQ ID NO:6, a V_{β} domain comprising or consisting of SEQ ID NO:13, a C_{α} domain comprising or consisting of SEQ ID NO:15, and a C_{β} domain comprising or consisting of SEQ ID NO:16 or SEQ ID NO:17.

In still other embodiments, the encoded binding protein comprises a V_{α} domain comprising or consisting of SEQ ID NO:7, a V_{β} domain comprising or consisting of SEQ ID NO:14, a C_{α} domain comprising or consisting of SEQ ID NO:15, and a C_{β} domain comprising or consisting of SEQ ID NO:16 or SEQ ID NO:17.

In certain embodiments, any of the disclosed encoded 5T4-specific binding proteins are each a T cell receptor (TCR), a chimeric antigen receptor (CAR), or an antigen-binding fragment of a TCR, any of which can be chimeric, humanized or human. In further embodiments, an antigen-binding fragment of a TCR is contained in a scTCR or a CAR. Methods for producing engineered TCRs are described in, for example, Bowerman et al., Mol. Immunol., 46(15):3000 (2009), the techniques of which are herein incorporated by reference. Methods for making CARs are described, for example, in U.S. Pat. Nos. 6,410,319; 7,446,191; U.S. Patent Publication No. 2010/065818; U.S. Pat. No. 8,822,647; PCT Publication No. WO 2014/031687; U.S. Pat. No. 7,514,537; and Brentjens et al., 2007, Clin. Cancer Res. 13:5426, the techniques of which are herein incorporated by reference. In certain embodiments, a binding protein encoded by a modified immune cell comprises a TCR, which is expressed on the cell surface, wherein the cell surface-expressed TCR is capable of more efficiently associating with a CD3 protein as compared to endogenous TCR. A TCR of this disclosure, when expressed on the surface of a T cell, may also have higher surface expression on the T cell as compared to endogenous TCR. In certain embodiments, a binding protein comprises a CAR, wherein the binding domain of the CAR comprises an antigen-specific TCR binding domain (see, e.g., Walseng et al., Scientific Reports 7:10713, 2017; the TCR CAR constructs and methods of which are incorporated by reference herein).

In some embodiments, an encoded 5T4-specific binding protein described herein may possess one or more amino acid substitutions, deletions, or additions relative to a naturally occurring binding protein (e.g., TCR). Conservative substitutions of amino acids are well known and may occur naturally or may be introduced when the binding protein or TCR is recombinantly produced. Amino acid substitutions, deletions, and additions may be introduced into a protein using mutagenesis methods known in the art (see, e.g., Sambrook et al., Molecular Cloning: A Laboratory Manual, 3d ed., Cold Spring Harbor Laboratory Press, N Y, 2001). Oligonucleotide-directed site-specific (or segment specific) mutagenesis procedures may be employed to provide an altered polynucleotide that has particular codons altered according to the substitution, deletion, or insertion desired. Alternatively, random or saturation mutagenesis techniques, such as alanine scanning mutagenesis, error prone polymerase chain reaction mutagenesis, and oligonucleotide-directed mutagenesis may be used to prepare immunogen polypeptide variants (see, e.g., Sambrook et al., supra).

A variety of criteria known to persons skilled in the art indicate whether an amino acid that is substituted at a particular position in a peptide or polypeptide is conservative (or similar). For example, a similar amino acid or a conservative amino acid substitution is one in which an amino acid residue is replaced with an amino acid residue having a similar side chain. Similar amino acids may be included in the following categories: amino acids with basic side chains (e.g., lysine, arginine, histidine); amino acids with acidic side chains (e.g., aspartic acid, glutamic acid); amino acids with uncharged polar side chains (e.g., glycine, asparagine, glutamine, serine, threonine, tyrosine, cysteine, histidine); amino acids with nonpolar side chains (e.g., alanine, valine, leucine, isoleucine, proline, phenylalanine, methionine, tryptophan); amino acids with beta-branched side chains (e.g., threonine, valine, isoleucine), and amino acids with aromatic side chains (e.g., tyrosine, phenylalanine, tryptophan). Proline, which is considered more difficult to classify, shares properties with amino acids that have aliphatic side chains (e.g., leucine, valine, isoleucine, and alanine). In certain circumstances, substitution of glutamine for glutamic acid or asparagine for aspartic acid may be considered a similar substitution in that glutamine and asparagine are amide derivatives of glutamic acid and aspartic acid, respectively. As understood in the art “similarity” between two polypeptides is determined by comparing the amino acid sequence and conserved amino acid substitutes thereto of the polypeptide to the sequence of a second polypeptide (e.g., using GENEWORKS, Align, the BLAST algorithm, or other algorithms described herein and practiced in the art).

In any of the embodiments described herein, an encoded binding protein can comprise a “signal peptide” (also known as a leader sequence, leader peptide, or transit peptide). Signal peptides target newly synthesized polypeptides to their appropriate location inside or outside the cell. A signal peptide may be removed from the polypeptide during or once localization or secretion is completed. Polypeptides that have a signal peptide are referred to herein as a “pre-protein” and polypeptides having their signal peptide removed are referred to herein as “mature” proteins or polypeptides. In certain embodiments, a binding protein of this disclosure comprises a mature Vβ domain, a mature Vα domain, or both. In some embodiments, a binding protein of this disclosure comprises a mature TCR β-chain, a mature TCR α-chain, or a mature TCR β-chain and a mature TCR α-chain.

Exemplary binding proteins and fusion proteins of this disclosure expressed by a cell may include a signal peptide (e.g., binding pre-proteins), and the cell may remove the signal peptide to generate a mature binding protein. In certain embodiments, a binding protein comprises two components, such as an a chain and a (3 chain, which can associate on the cell surface to form a functional binding protein. The two associated components may comprise mature proteins.

In any of the presently disclosed embodiments, a modified immune cell is capable of binding to a 5T4p17:HLA complex (e.g., expressed on the surface of a cell) with high avidity.

Host Cells

In certain embodiments, a polynucleotide encoding a binding protein specific for 5T4 is used to transfect/transduce a host cell (e.g., a T cell) for use in adoptive transfer therapy. Advances in TCR sequencing have been described (e.g., Robins et al., Blood 114:4099, 2009; Robins et al., Sci. Translat. Med. 2:47ra64, 2010; Robins et al., (September 10) J. Imm. Meth. Epub ahead of print, 2011; Warren et al., Genome Res. 21:790, 2011) and may be employed in the course of practicing embodiments according to the present disclosure. Similarly, methods for transfecting/transducing T cells with desired nucleic acids have been described (e.g., U.S. Patent Application Pub. No. US 2004/0087025) as have adoptive transfer procedures using T cells of desired antigen-specificity (e.g., Schmitt et al., Hum. Gen. 20:1240, 2009; Dossett et al., Mol. Ther. 17:742, 2009; Till et al., Blood 112:2261, 2008; Wang et al., Hum. Gene Ther. 18:712, 2007; Kuball et al., Blood 109:2331, 2007; US 2011/0243972; US 2011/0189141; Leen et al., Ann. Rev. Immunol. 25:243, 2007), such that adaptation of these methodologies to the presently disclosed embodiments is contemplated, based on the teachings herein, including those directed to high-affinity TCRs specific for 5T4 peptide antigens complexed with an HLA receptor.

Any suitable immune cell may be modified to include a heterologous polynucleotide of this disclosure, including, for example, a T cell, a NK cell, or a NK-T cell. In some embodiments, a modified immune cell comprises a CD4⁺ T cell, a CD8⁺ T cell, or both. Methods for transfecting/transducing T cells with desired nucleic acids have been described (e.g., U.S. Patent Application Pub. No. US 2004/0087025) as have adoptive transfer procedures using T cells of desired target-specificity (e.g., Schmitt et al., Hum. Gen. 20:1240, 2009; Dossett et al., Mol. Ther. 17:742, 2009; Till et al., Blood 112:2261, 2008; Wang et al., Hum. Gene Ther. 18:712, 2007; Kuball et al., Blood 109:2331, 2007; US 2011/0243972; US 2011/0189141; Leen et al., Ann. Rev. Immunol. 25:243, 2007).

Any appropriate method can be used to transfect or transduce the cells, for example, T cells, or to administer the nucleotide sequences or compositions of the present methods. Methods for delivering polynucleotides to host cells include, for example, use of cationic polymers, lipid-like molecules, and certain commercial products such as, for example, IN-VIVO-JET PEI. Other methods include ex vivo transduction, injection, electroporation, DEAE-dextran, sonication loading, liposome-mediated transfection, receptor-mediated transduction, microprojectile bombardment, transposon-mediated transfer, and the like. Still further methods of transfecting or transducing host cells employ vectors, described in further detail herein.

Modified immune cells as described herein may be functionally characterized using methodologies for assaying T cell activity, including determination of T cell binding, activation or induction and also including determination of T cell responses that are antigen-specific. Examples include determination of T cell proliferation, T cell cytokine release, antigen-specific T cell stimulation, MHC restricted T cell stimulation, CTL activity (e.g., by detecting ⁵¹Cr release from pre-loaded target cells), changes in T cell phenotypic marker expression, and other measures of T-cell functions. Procedures for performing these and similar assays may be found, for example, in Lefkovits (Immunology Methods Manual: The Comprehensive Sourcebook of Techniques, 1998). See, also, Current Protocols in Immunology; Weir, Handbook of Experimental Immunology, Blackwell Scientific, Boston, Mass. (1986); Mishell and Shigii (eds.) Selected Methods in Cellular Immunology, Freeman Publishing, San Francisco, Calif. (1979); Green and Reed, Science 281:1309 (1998) and references cited therein.

In certain examples, apparent affinity for a TCR is measured by assessing binding to various concentrations of MHC tetramers. “MHC-peptide tetramer staining” refers to an assay used to detect antigen-specific T cells, which features a tetramer of MHC molecules, each comprising an identical peptide having an amino acid sequence that is cognate (e.g., identical or related to) at least one antigen (e.g., 5T4), wherein the complex is capable of binding T cell receptors specific for the cognate antigen. Each of the MHC molecules may be tagged with a biotin molecule. Biotinylated MHC/peptides are tetramerized by the addition of streptavidin, which can be fluorescently labeled. The tetramer may be detected by flow cytometry via the fluorescent label. In certain embodiments, an MHC-peptide tetramer assay is used to detect or select enhanced affinity TCRs of the instant disclosure. In some examples, apparent K_{D} of a TCR is measured using 2-fold dilutions of labeled tetramers at a range of concentrations, followed by determination of binding curves by non-linear regression, apparent K_{D} being determined as the concentration of ligand that yielded half-maximal binding.

Levels of cytokines may be determined using methods described herein, such as ELISA, ELISPOT, intracellular cytokine staining, and flow cytometry and combinations thereof (e.g., intracellular cytokine staining and flow cytometry). Immune cell proliferation and clonal expansion resulting from an antigen-specific elicitation or stimulation of an immune response may be determined by isolating lymphocytes, such as circulating lymphocytes in samples of peripheral blood cells or cells from lymph nodes, stimulating the cells with antigen, and measuring cytokine production, cell proliferation and/or cell viability, such as by incorporation of tritiated thymidine or non-radioactive assays, such as MTT assays and the like. The effect of an immunogen described herein on the balance between a Th1 immune response and a Th2 immune response may be examined, for example, by determining levels of Th1 cytokines, such as IFN-γ, IL-12, IL-2, and TNF-0, and Type 2 cytokines, such as IL-4, IL-5, IL-9, IL-10, and IL-13.

In any of the foregoing embodiments, a modified immune cell that comprises a heterologous polynucleotide encoding a 5T4-specific binding protein can be a universal immune cell. A “universal immune cell” comprises an immune cell that has been modified to reduce or eliminate expression of one or more endogenous genes that encode a polypeptide product selected from PD-1, LAG-3, CTLA4, TIM3, TIGIT, an HLA molecule, a TCR molecule, or any combination thereof.

Without wishing to be bound by theory, certain endogenously expressed immune cell proteins may downregulate the immune activity of the modified immune cells (e.g., PD-1, LAG-3, CTLA4, TIGIT), or may interfere with the binding activity of a heterologously expressed binding protein of the present disclosure (e.g., an endogenous TCR that binds a non-5T4 antigen and interferes with the modified immune cell binding to a target cell that expresses a 5T4 antigen such as a 5T4p17 antigen (e.g., in a peptide:HLA complex.). Further, endogenous proteins (e.g., immune cell proteins, such as an HLA allele) expressed on a donor immune cell may be recognized as foreign by an allogeneic host, which may result in elimination or suppression of the modified donor immune cell by the allogeneic host.

Accordingly, decreasing or eliminating expression or activity of such endogenous genes or proteins can improve the activity, tolerance, or persistence of the modified immune cells in an autologous or allogeneic host setting, and allows universal administration of the cells (e.g., to any recipient regardless of HLA type). In certain embodiments, a universal immune cell is a donor cell (e.g., allogeneic) or an autologous cell. In certain embodiments, a modified immune cell (e.g., a universal immune cell) of this disclosure comprises a chromosomal gene knockout of one or more of a gene that encodes PD-1, LAG-3, CTLA4, TIM3, TIGIT, an HLA component (e.g., a gene that encodes an a1 macroglobulin, an a2 macroglobulin, an a3 macroglobulin, a (31 microglobulin, or a (32 microglobulin), or a TCR component (e.g., a gene that encodes a TCR variable region or a TCR constant region) (see, e.g., Torikai et al., Nature Sci. Rep. 6:21757 (2016); Torikai et al., Blood 119(24):5697 (2012); and Torikai et al., Blood 122(8):1341 (2013) the gene editing techniques, compositions, and adoptive cell therapies of which are herein incorporated by reference in their entirety).

As used herein, the term “chromosomal gene knockout” refers to a genetic alteration or introduced inhibitory agent in a host cell that prevents (e.g., reduces, delays, suppresses, or abrogates) production, by the host cell, of a functionally active endogenous polypeptide product. Alterations resulting in a chromosomal gene knockout can include, for example, introduced nonsense mutations (including the formation of premature stop codons), missense mutations, gene deletion, and strand breaks, as well as the heterologous expression of inhibitory nucleic acid molecules that inhibit endogenous gene expression in the host cell.

In certain embodiments, a chromosomal gene knock-out or gene knock-in is made by chromosomal editing of a host cell. Chromosomal editing can be performed using, for example, endonucleases. As used herein “endonuclease” refers to an enzyme capable of catalyzing cleavage of a phosphodiester bond within a polynucleotide chain. In certain embodiments, an endonuclease is capable of cleaving a targeted gene thereby inactivating or “knocking out” the targeted gene. An endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule may be used for a donor gene “knock-in”, for target gene “knock-out”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases, CRISPR-Cas nucleases, meganucleases, and megaTALs.

As used herein, a “zinc finger nuclease” (ZFN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90:2256-2260, 1993; Wolfe et al., J. Mol. Biol. 285:1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair. Alternatively, a DSB generated by a ZFN can result in knock out of target gene via repair by non-homologous end joining (NHEJ), which is an error-prone cellular repair pathway that results in the insertion or deletion of nucleotides at the cleavage site. In certain embodiments, a gene knockout comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a Fokl endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also known (see, e.g., U.S. Patent Publication No. US 2011/0301073, which atypical RVDs are incorporated by reference herein in their entirety). TALENs can be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair can introduce a transgene at the site of DSB providing homologous flanking sequences are present in the transgene. In certain embodiments, a gene knockout comprises an insertion, a deletion, a mutation or a combination thereof, and made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA:tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences can be introduced at the site of DSB via homology directed repair. The crRNA and tracrRNA can be engineered into a single guide RNA (sgRNA or gRNA) (see, e.g., Jinek et al., Science 337: 816-21, 2012). Further, the region of the guide RNA complementary to the target site can be altered or programed to target a desired sequence (Xie et al., PLOS One 9:e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference). In certain embodiments, a gene knockout comprises an insertion, a deletion, a mutation or a combination thereof, and made using a CRISPR/Cas nuclease system.

Exemplary gRNA sequences and methods of using the same to knock out endogenous genes that encode immune cell proteins include those described in Ren et al., Clin. Cancer Res. 23(9):2255-2266 (2017), the gRNAs, CAS9 DNAs, vectors, and gene knockout techniques of which are hereby incorporated by reference in their entirety.

As used herein, a “meganuclease,” also referred to as a “homing endonuclease,” refers to an endodeoxyribonuclease characterized by a large recognition site (double stranded DNA sequences of about 12 to about 40 base pairs). Meganucleases can be divided into five families based on sequence and structure motifs: LAGLIDADG (SEQ ID NO:159), GIY-YIG (SEQ ID NO:160), HNH, His-Cys box and PD-(D/E)XK (SEQ ID NO:161). Exemplary meganucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII, whose recognition sequences are known (see, e.g., U.S. Pat. Nos. 5,420,032 and 6,833,252; Belfort et al., Nucleic Acids Res. 25:3379-3388, 1997; Dujon et al., Gene 82:115-118, 1989; Perler et al., Nucleic Acids Res. 22:1125-1127, 1994; Jasin, Trends Genet. 12:224-228, 1996; Gimble et al., J. Mol. Biol. 263:163-180, 1996; Argast et al., J. Mol. Biol. 280:345-353, 1998).

In certain embodiments, naturally-occurring meganucleases may be used to promote site-specific genome modification of a target selected from PD-1, LAG3, TIM3, CTLA4, TIGIT, an HLA-encoding gene, or a TCR component-encoding gene. In other embodiments, an engineered meganuclease having a novel binding specificity for a target gene is used for site-specific genome modification (see, e.g., Porteus et al., Nat. Biotechnol. 23:967-73, 2005; Sussman et al., J. Mol. Biol. 342:31-41, 2004; Epinat et al., Nucleic Acids Res. 31:2952-62, 2003; Chevalier et al., Molec. Cell 10:895-905, 2002; Ashworth et al., Nature 441:656-659, 2006; Paques et al., Curr. Gene Ther. 7:49-66, 2007; U.S. Patent Publication Nos. US 2007/0117128; US 2006/0206949; US 2006/0153826; US 2006/0078552; and US 2004/0002092). In further embodiments, a chromosomal gene knockout is generated using a homing endonuclease that has been modified with modular DNA binding domains of TALENs to make a fusion protein known as a megaTAL. MegaTALs can be utilized to not only knock-out one or more target genes, but to also introduce (knock in) heterologous or exogenous polynucleotides when used in combination with an exogenous donor template encoding a polypeptide of interest.

In certain embodiments, a chromosomal gene knockout comprises an inhibitory nucleic acid molecule that is introduced into a host cell (e.g., an immune cell) comprising a heterologous polynucleotide encoding an antigen-specific receptor that specifically binds to a tumor associated antigen, wherein the inhibitory nucleic acid molecule encodes a target-specific inhibitor and wherein the encoded target-specific inhibitor inhibits endogenous gene expression (i.e., of PD-1, TIM3, LAGS, CTLA4, TIGIT, an HLA component, or a TCR component, or any combination thereof) in the host immune cell.

A chromosomal gene knockout can be confirmed directly by DNA sequencing of the host immune cell following use of the knockout procedure or agent. Chromosomal gene knockouts can also be inferred from the absence of gene expression (e.g., the absence of an mRNA or polypeptide product encoded by the gene) following the knockout.

In certain embodiments, a modified immune cell of the present disclosure is capable of specifically killing 50% or more of HLA-A2-expressing target cells in vitro, wherein the HLA-A2-expressing (e.g., HLA-A*0201-expressing) target cells are pulsed with a peptide or polypeptide comprising the amino acid sequence RLARLALVL (SEQ ID NO:64) at a concentration of about 1 nM to at least about 10 nM.

In certain embodiments, the modified immune cell is capable of producing a cytokine when contacted in vitro with HLA-A2-expressing (e.g., HLA-A*0201-expressing) target cells pulsed with a peptide or polypeptide comprising the amino acid sequence RLARLALVL (SEQ ID NO:64) at a concentration of about 1 nM to at least about 10 nM. In some embodiments, the cytokine comprises IFN-γ. In some embodiments, the cytokine comprises TNF-α.

In certain embodiments, the modified immune cell is capable of specifically killing a cancer cell in vitro, wherein the cancer cell expresses: (i) an HLA-A2 molecule; and (ii) a polypeptide comprising or consisting of the amino acid sequence RLARLALVL (SEQ ID NO:64). In some embodiments, the cancer cell is a renal cell carcinoma cell, a breast cancer cell, a colon cancer cell, or any combination thereof

Fusion Proteins

Also provided herein are fusion proteins that specifically bind to a 5T4₁₇₋₂₅ peptide antigen. In certain embodiments, a fusion protein comprises: (a) an extracellular component comprising a binding domain specific for a 5T4 antigen; (b) an intracellular component comprising an effector domain or a functional portion thereof; and (c) a transmembrane domain connecting the extracellular and intracellular components, wherein the fusion protein is capable of specifically binding to a 5T4 peptide (e.g., a 5T4 peptide:HLA complex, wherein the 5T4 peptide comprises or consists of (i) the amino acid sequence RLARLALVL (SEQ ID NO:64) or (ii) ALARLALVL (SEQ ID NO:168).

In certain embodiments, a 5T4-specific fusion protein includes a binding domain comprising: (a) a T cell receptor (TCR) α-chain variable (V_{α}) domain comprising a CDR3 amino acid sequence (CDR3α) having a net charge of about −0.01 to about −2.2 (i.e., including all values therebetween; e.g., including about −0.05, about −0.1, about −0.5, about −1.0, about −1.5, about −2.0, about −2.2, or the like). and a TCR β-chain variable (V_{β}) domain; or (b) a TCR V_{α} domain, and a TCR V_{β} domain comprising a CDR3 amino acid sequence (CDR3β) having a net charge of about −0.01 to about −2.2; or (c) a TCR V_{α} domain of (a), and a TCR V_{β} domain of (b).

In certain embodiments, (i) the CDR3α has a net charge of about −0.01 to about −2.2 and; (ii) the CDR3β has a net charge of about −0.01 to about −2.2.

In other embodiments, (i) the CDR3β has a net charge of about −0.05 to about −2.2; and (ii) the CDR3α has a net charge of about −0.05 to about −2.2.

In some embodiments, the CDR3α has a net charge of about −0.05 to about −2.2. In other embodiments, the CDR3α has a net charge of about −1.0 to about −2.2.

In some embodiments, the CDR3β has a net charge of about −0.05 to about −1.0. In other embodiments, the CDR3β has a net charge of about −1.0 to about −2.2.

In some embodiments, one of (i) the CDR3α or (ii) the CDR3β has a net charge of about −0.05; and the other of (i) the CDR3α or (ii) the CDR3β has a net charge of about −1.0. For example, in certain embodiments, the CDR3α has a net charge of about −0.05 and the CDR3β has a net charge of about −1.0. In other embodiments, the CDR3α has a net charge of about −1.0 and the CDR3β has a net charge of about −0.05.

In other embodiments, the CDR3α and the CDR3β each have a net charge of about −1.0. In further embodiments, the CDR3α and the CDR3β each have a net charge of about −1.05.

In still other embodiments, the CDR3α and the CDR3β each have a net charge of about −2.2. In further embodiments, the CDR3α and the CDR3β each have a net charge of about −2.05.

In any of the embodiments disclosed herein, the CDR3α and/or the CDR3β can have a net charge of an exemplary CDR3α or CDR3β as set forth in Table 1, or can have a net charge that is about the same as the net charge of an exemplary CDR3α or CDR3β (respectively) as set forth in Table 1. In some embodiments, an encoded binding protein of the present disclosure comprises a CDR3α and a CDR3β that have net charges that are about the same as the net charges of an exemplary clone as set forth in Table 1 (e.g., clone2-6B8; clone15-3F10; clone6-5G8; clone17-9B5; clone3-6C3; clone19-5C2; clone21-7A10).

As used herein, an “effector domain” or “immune effector domain” is an intracellular portion or domain of a fusion protein or receptor that can directly or indirectly promote an immune response in a cell when receiving an appropriate signal. In certain embodiments, an effector domain is from a immune cell protein or portion thereof or immune cell protein complex that receives a signal when bound (e.g., CD3), or when the immune cell protein or portion thereof or immune cell protein complex binds directly to a target molecule and triggers signal transduction from the effector domain in an immune cell.

An effector domain may directly promote a cellular response when it contains one or more signaling domains or motifs, such as an Intracellular Tyrosine-based Activation Motif (ITAM), such as those found in costimulatory molecules. Without wishing to be bound by theory, it is believed that ITAMs are important for T cell activation following ligand engagement by a T cell receptor or by a fusion protein comprising a T cell effector domain. In certain embodiments, the intracellular component or functional portion thereof comprises an ITAM. Exemplary immune effector domains include those from, CD3ε, CD3δ, CD3ζ, CD25, CD79A, CD79B, CARD11, DAP10, FcRα, FcRβ, FcRγ, Fyn, HVEM, ICOS, Lck, LAG3, LAT, LRP, NKG2D, NOTCH1, NOTCH2, NOTCH3, NOTCH4, Wnt, ROR2, Ryk, SLAMF1, Slp76, pTα, TCRα, TCRβ, TRIM, Zap70, PTCH2, or any combination thereof. In certain embodiments, an effector domain comprises a lymphocyte receptor signaling domain (e.g., CD3ζ or a functional portion or variant thereof).

In further embodiments, the intracellular component of the fusion protein comprises a costimulatory domain or a functional portion thereof selected from CD27, CD28, 4-1BB (CD137), OX40 (CD134), CD2, CD5, ICAM-1 (CD54), LFA-1 (CD11a/CD18), ICOS (CD278), GITR, CD30, CD40, BAFF-R, HVEM, LIGHT, MKG2C, SLAMF7, NKp80, CD160, B7-H3, a ligand that specifically binds with CD83, or a functional variant thereof, or any combination thereof. In certain embodiments, the intracellular component comprises a CD28 costimulatory domain or a functional portion or variant thereof (which may optionally include a LL→GG mutation at positions 186-187 of the native CD28 protein (see Nguyen et al., Blood 102:4320, 2003)), a 4-1BB costimulatory domain or a functional portion or variant thereof, or both.

In certain embodiments, an effector domain comprises a CD3ζ endodomain or a functional (e.g., signaling) portion thereof, or a functional variant thereof. In further embodiments, an effector domain comprises a CD27 endodomain or a functional (e.g., signaling) portion thereof, or a functional variant thereof. In further embodiments, an effector domain comprises a CD28 endodomain or a functional (e.g., signaling) portion thereof, or a functional variant thereof. In still further embodiments, an effector domain comprises a 4-1BB endodomain or a functional (e.g., signaling) portion thereof, or a functional variant thereof. In further embodiments, an effector domain comprises an OX40 endodomain or a functional (e.g., signaling) portion thereof, or a functional variant thereof. In further embodiments, an effector domain comprises a CD2 endodomain or a functional (e.g., signaling) portion thereof, or a functional variant thereof. In further embodiments, an effector domain comprises a CD5 endodomain or a functional (e.g., signaling) portion thereof, or a functional variant thereof. In further embodiments, an effector domain comprises an ICAM-1 endodomain or a functional (e.g., signaling) portion thereof, or a functional variant thereof. In further embodiments, an effector domain comprises a LFA-1 endodomain or a functional (e.g., signaling) portion thereof, or a functional variant thereof. In further embodiments, an effector domain comprises an ICOS endodomain or a functional (e.g., signaling) portion thereof, or a functional variant thereof.

An extracellular component and an intracellular component of the present disclosure are connected by a transmembrane domain. A “transmembrane domain,” as used herein, is a portion of a transmembrane protein that can insert into or span a cell membrane. Transmembrane domains have a three-dimensional structure that is thermodynamically stable in a cell membrane and generally range in length from about 15 amino acids to about 30 amino acids. The structure of a transmembrane domain may comprise an alpha helix, a beta barrel, a beta sheet, a beta helix, or any combination thereof. In certain embodiments, the transmembrane domain comprises or is derived from a known transmembrane protein (e.g., a CD4 transmembrane domain, a CD8 transmembrane domain, a CD27 transmembrane domain, a CD28 transmembrane domain, or any combination thereof).

In certain embodiments, the extracellular component of the fusion protein further comprises a linker disposed between the binding domain and the transmembrane domain. As used herein when referring to a component of a fusion protein that connects the binding and transmembrane domains, a “linker” may be an amino acid sequence having from about two amino acids to about 500 amino acids, which can provide flexibility and room for conformational movement between two regions, domains, motifs, fragments, or modules connected by the linker. For example, a linker of the present disclosure can position the binding domain away from the surface of a host cell expressing the fusion protein to enable proper contact between the host cell and a target cell, antigen binding, and activation (Patel et al., Gene Therapy 6: 412-419, 1999). Linker length may be varied to maximize antigen recognition based on the selected target molecule, selected binding epitope, or antigen binding domain seize and affinity (see, e.g., Guest et al., J. Immunother. 28:203-11, 2005; PCT Publication No. WO 2014/031687). Exemplary linkers include those having a glycine-serine amino acid chain having from one to about ten repeats of GlyxSery, wherein x and y are each independently an integer from 0 to 10, provided that x and y are not both 0 (e.g., (Gly₄Ser)₂ (SEQ ID NO:163), (Gly₃Ser)₂ (SEQ ID NO:164), Gly₂Ser, or a combination thereof, such as ((Gly₃Ser)₂Gly₂Ser) (SEQ ID NO:165).

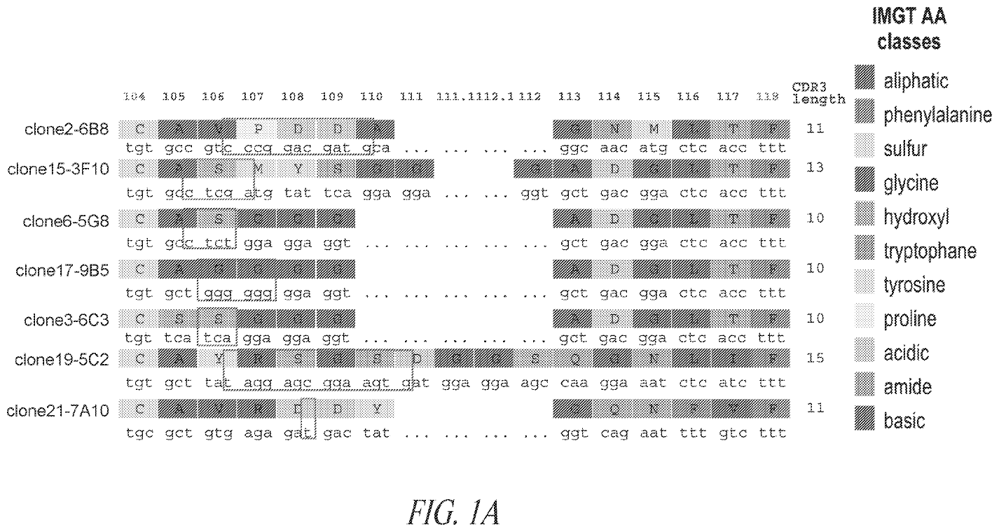

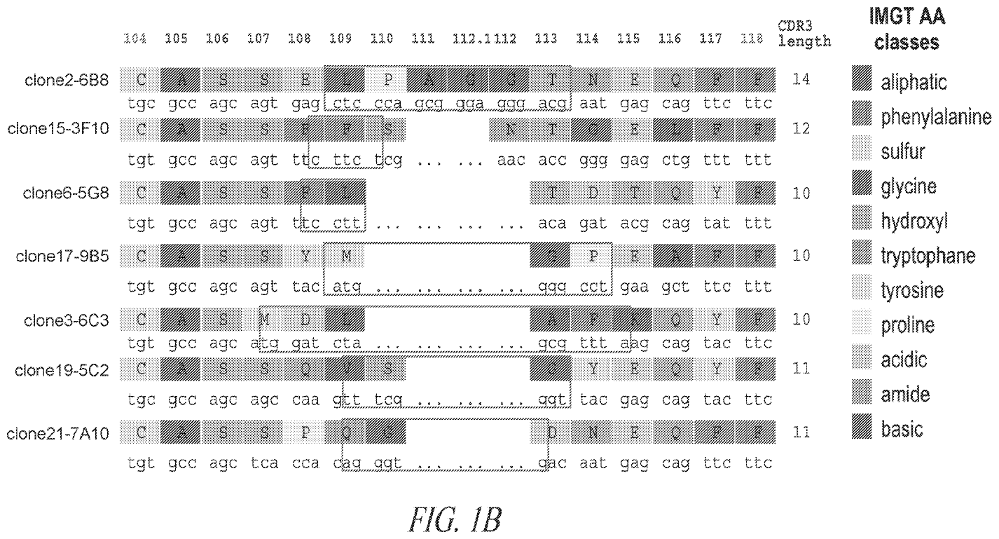

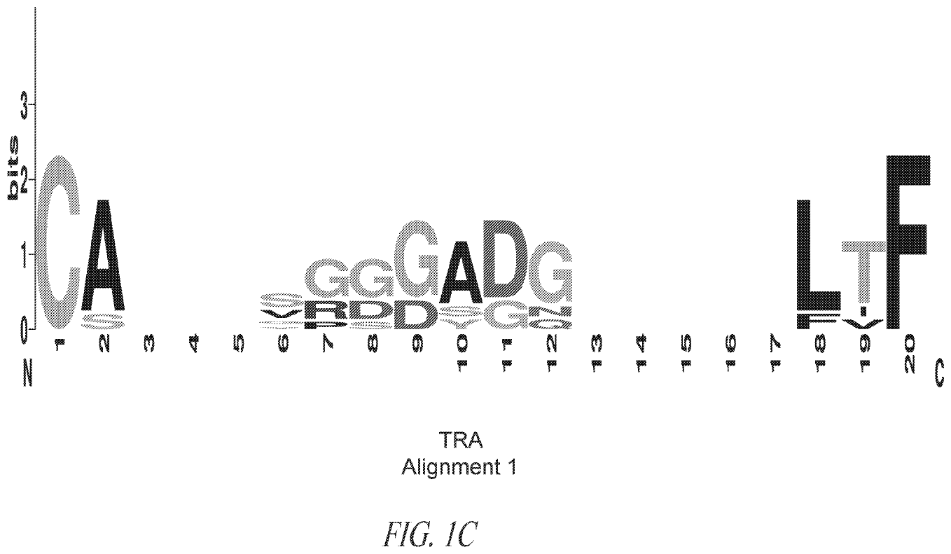

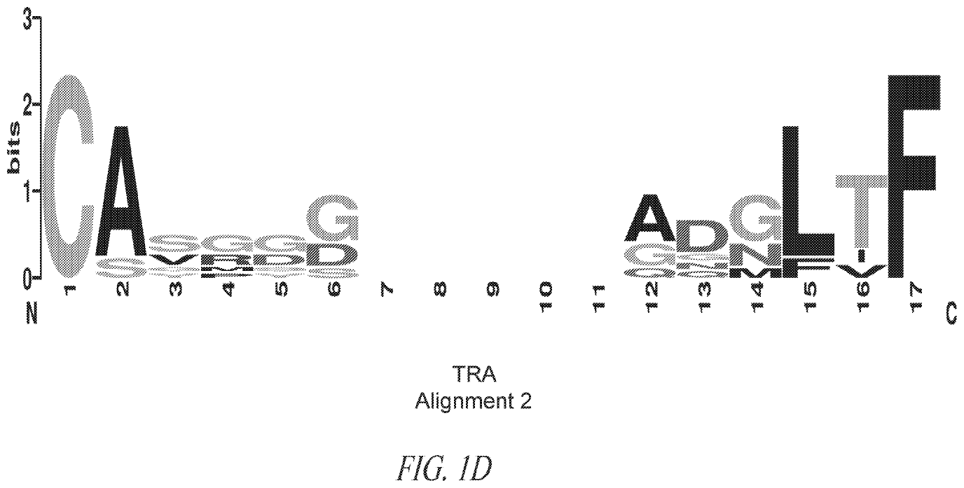

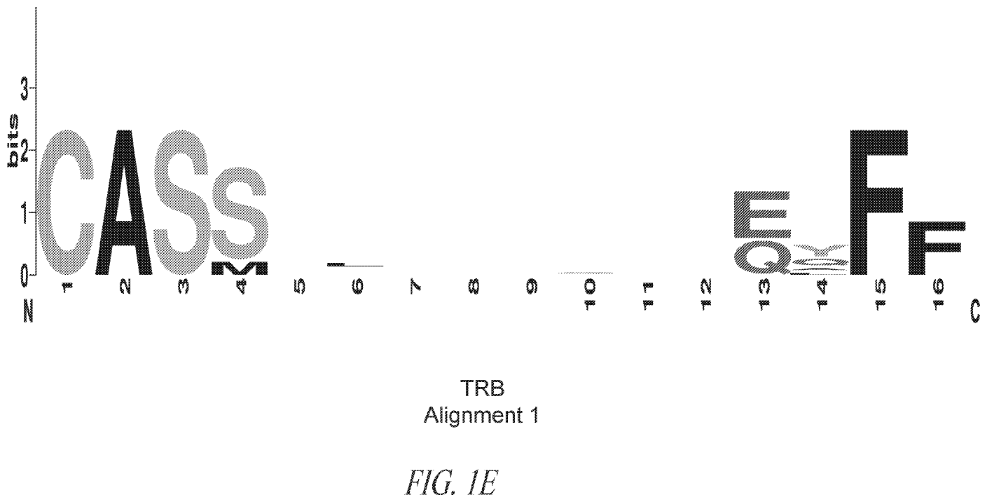

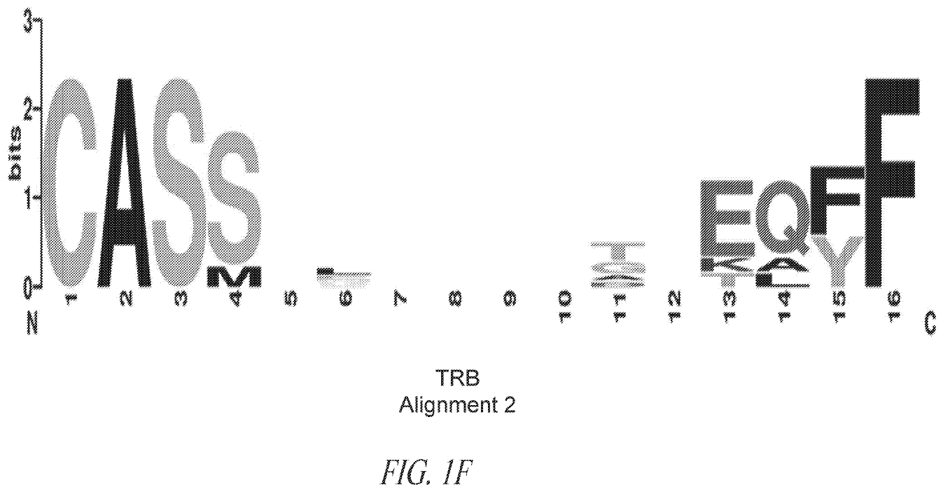

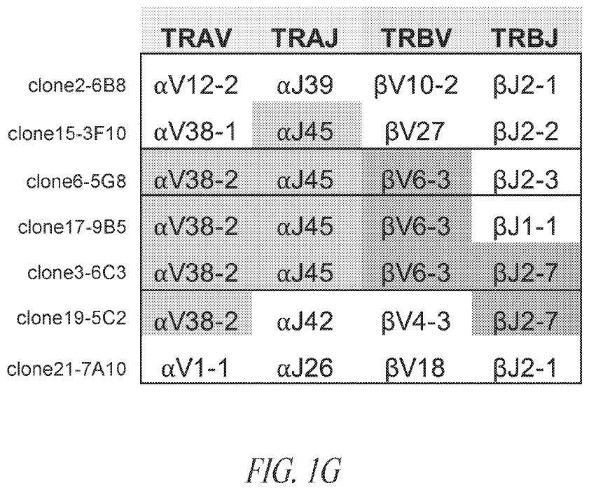

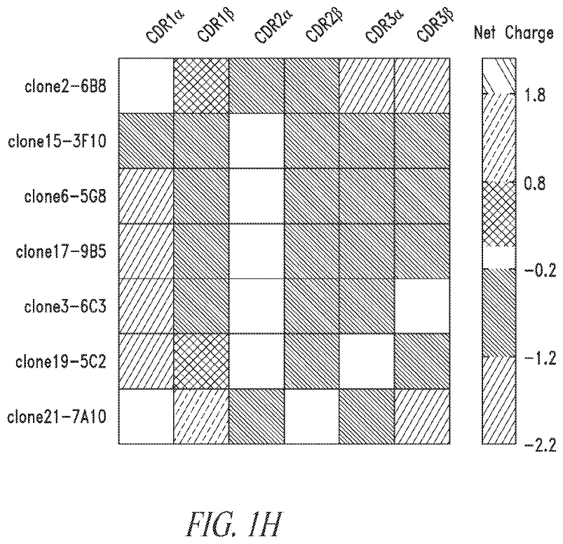

In some embodiments, the fusion protein comprises a binding domain encoded by a TRAV allele and a TRAJ allele as disclosed herein and exemplified in FIG. 1G.

In some embodiments, TRAV allele is αV38-2 and the TRAJ allele is αJ45.

In some embodiments, the TRBV allele is βV6-3.

In some embodiments, the TRBJ allele is βJ2-7.

In particular embodiments: (i) the TRAV allele is αV38-2; (ii) the TRAJ allele is αJ45; (iii) the TRBV allele is βV6-3; and (iv) the TRBJ allele is optionally βJ2-7.

In certain embodiments, a 5T4-specific binding protein of the present disclosure comprises a CDR3α having the consensus sequence of CX₁X₂GGGX₃X₄GX₅X₆F (SEQ ID NO:173), wherein: X₁ is A, V, L, I, G, S, or T; X₂ is A, V, G, L, I, S, T, P, or Y; X₃ is A, V, G, L, I, or S; X₄ is D or E; X₅ is L, I, V, or A; and X₆ is T or S. In further embodiments, X₁ is A or S; X₂ is S or G; X₃ is A; X₄ is D; X₅ is L; and/or X₆ is T.

In certain embodiments, a 5T4-specific fusion protein of the present disclosure comprises a binding domain comprising a CDR3β having the consensus sequence CX₇X₈X₉X₁₀X₁₁X₁₂X₁₃X₁₄X₁₅X₁₆X₁₇F (SEQ ID NO:174), wherein: X₇ is A, V, I, or L; X₈ is S or T; X₉ is S, T, or M; X₁₀ is F, D, E, Y, Q, or P; X₁₁ is L, I, V, A, M, F, or Q; X₁₂ is from 0 to 4 amino acids and optionally consists of: G, S, SN, or PAGG; X₁₃ is T, S, G, A, L, I, V, D, or E; X₁₄ is N, D, Y, F, P, or G; X₁₅ is E, T, or K; X₁₆ is Q, N, A, L, I, or V; and X₁₇ is F or Y. In further embodiments, X₇ is A; X₈ is 5; X₉ is 5; X₁₅ is E; and/or X₁₆ is Q. In particular embodiments, a 5T4-specific fusion protein of the present disclosure comprises a binding domain comprising (i) a CDR3α having the consensus sequence of CX₁X₂GGGX₃X₄GX₅X₆F (SEQ ID NO:173), wherein: X₁ is A or S; X₂ is S or G; X₃ is A; X₄ is D; X₅ is L; and/or X₆ is T; and (ii) a CDR3p having the consensus sequence CX₇X₈X₉X₁₀X₁₁X₁₂X₁₃X₁₄X₁₅X₁₆X₁₇F (SEQ ID NO:174) as disclosed herein, wherein: X₇ is A; X₈ is 5; X₉ is 5; X₁₅ is E; and/or X₁₆ is Q.

In any of the herein disclosed embodiments, the encoded binding protein is capable of binding to a peptide:HLA-A2 (also referred to herein as HLA-A*0201) complex in which the peptide comprises or consists of the amino acid sequence ALARLALVL (SEQ ID NO:168).

In certain embodiments, the fusion protein comprises (a) an extracellular component that includes a binding domain comprising (i) a T cell receptor (TCR) a chain variable (V_{α}) domain having a CDR3 amino acid sequence shown in any one of SEQ ID NOS:49-55, and a TCR β chain variable (V_{β}) domain, (ii) a V_{α} domain, and a V_{β} domain having a CDR3 amino acid sequence shown in any one of SEQ ID NOS:56-62, or (iii) a Vα domain of (i), and a Vβ domain of (ii); (b) an intracellular component comprising an effector domain or a functional portion thereof; and (c) a transmembrane domain connecting the extracellular and intracellular components, wherein the fusion protein is capable of specifically binding to a 5T4 peptide. For example, in some embodiments, a fusion protein is capable of specifically binding to a 5T4:HLA complex (e.g., a RLARLALVL (SEQ ID NO:64):HLA-A*201 complex expressed on a surface of a cell).

In some embodiments, the binding domain of the fusion protein is capable of specifically binding to a RLARLALVL (SEQ ID NO:64):human leukocyte antigen (HLA) complex with a K_{d} less than or equal to about 10⁻⁸M.

In some embodiments, the binding domain is capable of specifically binding to a 5T4 peptide:HLA complex on a cell surface independent of CD8 or in the absence of CD8.

In some embodiments, the binding domain specifically binds to a RLARLALVL (SEQ ID NO:64):HLA-A*201 complex and optionally specifically binds to a ALARLALVL (SEQ ID NO:168):HLA-A*201 complex.

In any of the embodiments disclosed herein, the V_{α} domain and the V_{β} domain of a fusion protein binding domain can each comprise a CDR1 amino acid sequence (CDR1α; CDR1β) and a CDR2 amino acid sequence (CDR2α; CDR2β), wherein: (i) the CDR1α has a net charge of about −2.0 to about 0 (e.g., about −2.0, about −1.5, about −1.0, about −0.5, or about 0); (ii) the CDR1β has a net charge of about −1.0 to about 1.5 (e.g., about −1.0, about −0.5, about 0, about 0.5, about 1.0. or about 1.5); (iii) the CDR2α has a net charge of about 0 to about −1.0 (e.g., about 0, about −0.5, or about −1.0); and/or (iv) the CDR2β has a net charge of about 0 to about −1.0.

In certain embodiments, the CDR1α has a net charge of about 0 to about −1.0. In other embodiments, the CDR1α has a net charge of about −1.0 to about −2.0.

In certain embodiments, the CDR1β has a net charge of about 0 to about 1.5. In further embodiments, the CDR1β has a net charge of about 0.01 to about 1.2. In some embodiments, the CDR1β has a net charge of about −1.0.

In certain embodiments, the CDR2α has a net charge of about 0 to about −1.0. In some embodiments, the CDR2α has a net charge of about 0. In some embodiments, the CDR2α has a net charge of about −1.0.

In certain embodiments, the CDR2β has a net charge of about 0 to about −1.0. In some embodiments, the CDR2β has a net charge of about 0. In some embodiments, the CDR2β has a net charge of about −1.0.

In any of the embodiments disclosed herein, the CDR1α, CDR1β, CDR2α, and/or CDR2β, can comprise a net charge of an exemplary CDR1α, CDR1β, CDR2α, and/or CDR2β as set forth in Table 2, or can have a net charge that is about the same as the net charge of an exemplary CDR1α, CDR1β, CDR2α, and/or CDR2β (respectively) as set forth in Table 2. In some embodiments, an encoded binding protein of the present disclosure comprises a CDR1α, CDR1β, CDR2α, and a CDR2β that have net charges that are about the same as the net charges of an exemplary clone as set forth in Table 2 (e.g., clone2-6B8; clone15-3F10; clone6-5G8; clone17-9B5; clone3-6C3; clone19-5C2; clone21-7A10).

In any of the embodiments disclosed herein, a 5T4-specific fusion protein can comprise CDRs (e.g., 1α, 1β, 2α, 2β, 3α, 3β) having net charges that are about the same as the net charges of CDRs of the exemplary clones set forth in Table 1 and Table 2.

In some embodiments, the binding domain comprises a V_{α} domain that is at least about 90% identical to the amino acid sequence shown in any one of SEQ ID NOS:1-7, and comprises a V_{β} domain that is at least about 90% identical to the amino acid sequence shown in any one of SEQ ID NOS:8-14, provided that (a) at least three or four of the CDRs have no change in sequence, wherein the CDRs that do have sequence changes have only up to two to four amino acid substitutions, insertions, deletions, or a combination thereof, and (b) the fusion protein is capable of specifically binding to the 5T4 peptide:HLA complex.

In some embodiments, the fusion protein specifically binding to the 5T4 peptide:HLA complex on a cell surface, wherein the specific binding is independent, or in the absence, of CD8.

In some embodiments, the V_{α} domain comprises or consists of the amino acid sequence shown in any one of SEQ ID NOS:1-7. In some embodiments, the V_{β} domain comprises or consists of the amino acid sequence shown in any one of SEQ ID NOS:8-14.

In any of the herein disclosed embodiments, the fusion protein can comprise a V_{α} domain comprising or consisting of the amino acid sequence shown in any one of SEQ ID NOS:1-7 and a V_{β} domain comprising or consisting of the amino acid sequence shown in any one of SEQ ID NOS:8-14.

In some embodiments, the fusion protein further comprises an α-chain constant (C_{α}) domain connected to the V_{α} domain and the C_{α} domain has at least 90% sequence identity to the amino acid sequence of SEQ ID NO:15.

In some embodiments, the extracellular component further comprises a β-chain constant (C_{β}) domain connected to the V_{β} domain, wherein the C_{β} domain has at least 90% sequence identity to the amino acid sequence shown in SEQ ID NO:16 or 17.

In certain embodiments, the extracellular component of the fusion protein comprises a V_{α} domain comprising or consisting of SEQ ID NO:1, a V_{β} domain comprising or consisting of SEQ ID NO:8, a C_{α} domain comprising or consisting of SEQ ID NO:15, and a C_{β} domain comprising or consisting of SEQ ID NO:16 or SEQ ID NO:17.

In certain embodiments, the extracellular component of the fusion protein comprises a V_{α} domain comprising or consisting of SEQ ID NO:2, a V_{β} domain comprising or consisting of SEQ ID NO:9, a C_{α} domain comprising or consisting of SEQ ID NO:15, and a C_{β} domain comprising or consisting of SEQ ID NO:16 or SEQ ID NO:17.

In certain embodiments, the extracellular component of the fusion protein comprises a V_{α} domain comprising or consisting of SEQ ID NO:3, a V_{β} domain comprising or consisting of SEQ ID NO:10, a C_{α} domain comprising or consisting of SEQ ID NO:15, and a C_{β} domain comprising or consisting of SEQ ID NO: 16 or SEQ ID NO:17.

In certain embodiments, the extracellular component of the fusion protein comprises a V_{α} domain comprising or consisting of SEQ ID NO:4, a V_{β} domain comprising or consisting of SEQ ID NO:11, a C_{α} domain comprising or consisting of SEQ ID NO:15, and a C_{β} domain comprising or consisting of SEQ ID NO:16 or SEQ ID NO:17.

In certain embodiments, the extracellular component of the fusion protein comprises a V_{α} domain comprising or consisting of SEQ ID NO:5, a V_{β} domain comprising or consisting of SEQ ID NO:12, a C_{α} domain comprising or consisting of SEQ ID NO:15, and a C_{β} domain comprising or consisting of SEQ ID NO:16 or SEQ ID NO:17.

In certain embodiments, the extracellular component of the fusion protein comprises a V_{α} domain comprising or consisting of SEQ ID NO:6, a V_{β} domain comprising or consisting of SEQ ID NO:13, a C_{α} domain comprising or consisting of SEQ ID NO:15, and a C_{β} domain comprising or consisting of SEQ ID NO:16 or SEQ ID NO:17.

In certain embodiments, the extracellular component of the fusion protein comprises a V_{α} domain comprising or consisting of SEQ ID NO:7, a V_{β} domain comprising or consisting of SEQ ID NO:14, a C_{α} domain comprising or consisting of SEQ ID NO:15, and a C_{β} domain comprising or consisting of SEQ ID NO:16 or SEQ ID NO:17.

In some embodiments, the fusion protein comprises an engineered T cell receptor (TCR), an antigen-binding fragment of a TCR, or a chimeric antigen receptor (CAR).

In some embodiments, the TCR, CAR, or antigen-binding fragment of a TCR is chimeric, humanized or human.

In some embodiments, the antigen-binding fragment of the TCR comprises a single chain TCR (scTCR). In some embodiments, the binding protein comprises a CAR. In some embodiments, the binding protein comprises a TCR.

A 5T4-specific binding protein or fusion protein of the present disclosure may, in some embodiments, be coupled to a cytotoxic or detectable agent. In certain embodiments, a fusion protein is expressed on the surface of a host cell (e.g., an immune cell such as a CD8⁺ or CD4⁺ T cell that comprises a polynucleotide encoding the fusion protein).

In another aspect, compositions are provided that comprise a 5T4-specific binding protein or fusion protein or host cell according to any one of the presently disclosed embodiments and a pharmaceutically acceptable carrier, diluent, or excipient. Methods useful for isolating and purifying recombinantly produced soluble TCR, by way of example, may include obtaining supernatants from suitable host cell/vector systems that secrete the recombinant soluble TCR into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant soluble TCR described herein include batch cell culture, which is monitored and controlled to maintain appropriate culture conditions. Purification of the soluble TCR may be performed according to methods described herein and known in the art and that comport with laws and guidelines of domestic and foreign regulatory agencies.

### Polynucleotides and Vectors

In another aspect, isolated or recombinant polynucleotides are provided herein, wherein a polynucleotide encodes a binding protein of the present disclosure (e.g., immunoglobulin superfamily binding protein, such as a TCR, scTCR, or CAR) specific for 5T4, and wherein the polynucleotide is codon optimized for expression in a host cell (e.g., an immune cell of the present disclosure). Polynucleotides that encode fusion proteins of this disclosure are also provided herein.

In some embodiments, a polynucleotide of the present disclosure comprises a polynucleotide having at least 70%, 75%, 80%, 85%, 90%, 91%, 92%, 93%, 94%, 95%, 96%, 97%, 98%, 99%, or 100% sequence identity with the polynucleotide sequence of any one of SEQ ID NOS:18-48, 65-70, and 112-146.

Also provided are vectors (e.g., expression vectors) that comprise a polynucleotide of this disclosure, wherein the polynucleotide is operatively associated or operably linked with an expression control sequence (e.g., a promoter). Construction of an expression vector to produce a binding protein or fusion protein specific for a 5T4 peptide of this disclosure can be made using restriction endonuclease digestion, ligation, transformation, plasmid purification, DNA sequencing, or a combination thereof, as described in, for example, Sambrook et al. (1989 and 2001 editions; Molecular Cloning: A Laboratory Manual, Cold Spring Harbor Laboratory Press, NY) and Ausubel et al. (Current Protocols in Molecular Biology, 2003). For efficient transcription and translation, a polynucleotide contained in an expression construct includes at least one appropriate expression control sequence (also called a regulatory sequence), such as a leader sequence and particularly a promoter operably (i.e., operatively) linked to the nucleotide sequence encoding the binding protein of this disclosure.

As one of skill in the art will recognize, a nucleic acid may be a single- or a double-stranded DNA, cDNA or RNA in any form, and may include a positive and a negative strand of the nucleic acid which complement each other, including anti-sense DNA, cDNA and RNA. Also included are siRNA, microRNA, RNA-DNA hybrids, ribozymes, and other various naturally occurring or synthetic forms of DNA or RNA.

Standard techniques may be used for recombinant (i.e., engineered) DNA, peptide and oligonucleotide synthesis, immunoassays and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well-known in the art and as described in various general and more specific references in microbiology, molecular biology, biochemistry, molecular genetics, cell biology, virology and immunology techniques that are cited and discussed throughout the present specification. See, e.g., Sambrook et al., Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3^{rd} Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and C C Blackwell, eds., 1986); Roitt, Essential Immunology, 6th Edition, (Blackwell Scientific Publications, Oxford, 1988); Embryonic Stem Cells: Methods and Protocols (Methods in Molecular Biology) (Kurstad Turksen, Ed., 2002); Embryonic Stem Cell Protocols: Volume I: Isolation and Characterization (Methods in Molecular Biology) (Kurstad Turksen, Ed., 2006); Embryonic Stem Cell Protocols: Volume II: Differentiation Models (Methods in Molecular Biology) (Kurstad Turksen, Ed., 2006); Human Embryonic Stem Cell Protocols (Methods in Molecular Biology) (Kursad Turksen Ed., 2006); Mesenchymal Stem Cells: Methods and Protocols (Methods in Molecular Biology) (Darwin J. Prockop, Donald G. Phinney, and Bruce A. Bunnell Eds., 2008); Hematopoietic Stem Cell Protocols (Methods in Molecular Medicine) (Christopher A. Klug, and Craig T. Jordan Eds., 2001); Hematopoietic Stem Cell Protocols (Methods in Molecular Biology) (Kevin D. Bunting Ed., 2008) Neural Stem Cells: Methods and Protocols (Methods in Molecular Biology) (Leslie P. Weiner Ed., 2008).

In certain embodiments, the instant disclosure provides an isolated polynucleotide encoding a binding protein, comprising a (i) polynucleotide encoding an α-chain variable (V_{α}) domain having a CDR3 amino acid sequence of any one of SEQ ID NOS:49-55, wherein the polynucleotide encoding the V_{α} domain has at least 70%, 75%, 76%, 77%, 78%, 79%, 80%, 85%, 90%, 91%, 92%, 93%, 94%, 95%, 96%, 97%, 98%, 99%, or 100% sequence identity with the polynucleotide sequence of any one of SEQ ID NOS:32-38; (ii) a polynucleotide encoding a β-chain variable (V_{β}) domain having a CDR3 amino acid sequence of any one of SEQ ID NOS:56-62, wherein the polynucleotide encoding the V_{β} domain has at least 70%, 75%, 76%, 77%, 78%, 79%, 80%, 85%, 90%, 91%, 92%, 93%, 94%, 95%, 96%, 97%, 98%, 99%, or 100% sequence identity with the polynucleotide sequence of any one of SEQ ID NOS:39-45, or (iii) a polynucleotide of (i) and a polynucleotide of (ii). In some embodiments, the encoded V_{α} domain has at least 80%, 85%, 90%, 91%, 92%, 93%, 94%, 95%, 96%, 97%, 98%, 99%, or 100% sequence identity to the amino acid sequence of any one of SEQ ID NOS:1-7, the encoded V_{β} domain has at least 80%, 85%, 90%, 91%, 92%, 93%, 94%, 95%, 96%, 97%, 98%, 99%, or 100% sequence identity to the amino acid sequence of any one of SEQ ID NOS:8-14, or any combination thereof. In further embodiments, the V_{α} domain-encoding polynucleotide further comprises a polynucleotide encoding an α-chain constant domain having at least 70%, 75%, 76%, 77%, 78%, 79%, 80%, 85%, 90%, 91%, 92%, 93%, 94%, 95%, 96%, 97%, 98%, 99%, or 100% sequence identity with the amino acid sequence of SEQ ID NO:15 and the polynucleotide encoding the α-chain constant domain has at least 70%, 75%, 76%, 77%, 78%, 79%, 80%, 85%, 90%, 91%, 92%, 93%, 94%, 95%, 96%, 97%, 98%, 99%, or 100% sequence identity with the polynucleotide sequence of SEQ ID NO:46, the V_{β} domain encoding polynucleotide further comprises a polynucleotide encoding a β-chain constant domain having at least 70%, 75%, 76%, 77%, 78%, 79%, 80%, 85%, 90%, 91%, 92%, 93%, 94%, 95%, 96%, 97%, 98%, 99%, or 100% sequence identity with the amino acid sequence of SEQ ID NO:16 or 17 and the polynucleotide encoding the β-chain constant domain has at least 70%, 75%, 76%, 77%, 78%, 79%, 80%, 85%, 90%, 91%, 92%, 93%, 94%, 95%, 96%, 97%, 98%, 99%, or 100% sequence identity with the polynucleotide sequence of SEQ ID NO:47 or 48, or any combination thereof.

In certain embodiments, any of the aforementioned isolated polynucleotides encoding a binding protein has a V_{α} domain-encoding polynucleotide that is contained in a polynucleotide encoding a TCR α-chain having at least 70%, 75%, 76%, 77%, 78%, 79%, 80%, 85%, 90%, 91%, 92%, 93%, 94%, 95%, 96%, 97%, 98%, 99%, or 100% sequence identity with the amino acid sequence of any one of SEQ ID NOS:77-83 and the polynucleotide encoding the TCR α-chain has at least 70%, 75%, 76%, 77%, 78%, 79%, 80%, 85%, 90%, 91%, 92%, 93%, 94%, 95%, 96%, 97%, 98%, 99%, or 100% sequence identity with the polynucleotide sequence of any one of SEQ ID NOS:112-118; and a V_{β} domain encoding polynucleotide is contained in a polynucleotide encoding a TCR β-chain having at least 70%, 75%, 76%, 77%, 78%, 79%, 80%, 85%, 90%, 91%, 92%, 93%, 94%, 95%, 96%, 97%, 98%, 99%, or 100% sequence identity with the amino acid sequence of any one of SEQ ID NOS:84-97 and the polynucleotide encoding the TCR β-chain has at least 70%, 75%, 76%, 77%, 78%, 79%, 80%, 85%, 90%, 91%, 92%, 93%, 94%, 95%, 96%, 97%, 98%, 99%, or 100% sequence identity with the polynucleotide sequence of any one of SEQ ID NOS:119-132.

In particular embodiments, any of the aforementioned isolated polynucleotide embodiments encoding a binding protein have structure from 5′-end to 3′-end of ([V_{β} domain encoding polynucleotide]-[self-cleaving peptide]-[V_{α} domain encoding polynucleotide]). In further embodiments, the encoded V_{β} domain, self-cleaving peptide, and V_{α} domain has at least 80%, 85%, 90%, 91%, 92%, 93%, 94%, 95%, 96%, 97%, 98%, 99%, or 100% sequence identity with the amino acid sequence of any one of SEQ ID NOS:98-111. In still further embodiments, the polynucleotide encoding the ([V_{β} domain]-[self-cleaving peptide]-[V_{α} domain]) has at least 70%, 75%, 76%, 77%, 78%, 79%, 80%, 85%, 90%, 91%, 92%, 93%, 94%, 95%, 96%, 97%, 98%, 99%, or 100% sequence identity with the polynucleotide sequence of any one of SEQ ID NOS:133-146.

In other embodiments, any of the aforementioned isolated polynucleotide embodiments encoding a binding protein have structure from 5′-end to 3′-end of ([V_{α} domain encoding polynucleotide]-[self-cleaving peptide]-[V_{β} domain encoding polynucleotide])

In certain embodiments, the instant disclosure provides an isolated polynucleotide encoding a binding protein, comprising a (i) polynucleotide encoding an α-chain variable (V_{α}) domain having a CDR3 amino acid sequence of any one of SEQ ID NOS:49-55, wherein the polynucleotide encoding the V_{α} domain is at least 75% or at least 79% or at least 80% identical to the polynucleotide sequence of any one of SEQ ID NOS:32-38; (ii) a polynucleotide encoding a β-chain variable (V_{β}) domain having a CDR3 amino acid sequence of any one of SEQ ID NOS:56-62, wherein the polynucleotide encoding the V_{β} domain is at least 80% identical to the polynucleotide sequence of any one of SEQ ID NOS:39-45, or (iii) a polynucleotide of (i) and a polynucleotide of (ii). In some embodiments, the encoded V_{α} domain has at least 90% sequence identity to the amino acid sequence of any one of SEQ ID NOS:1-7, the encoded V_{β} domain has at least 90% sequence identity to the amino acid sequence of any one of SEQ ID NOS:8-14, or any combination thereof. In further embodiments, the V_{α} domain-encoding polynucleotide comprises or consists of the polynucleotide sequence of any one of SEQ ID NOS:32-38, the V_{β} domain-encoding polynucleotide comprises or consists of the polynucleotide sequence of any one of SEQ ID NOS:39-45, or any combination thereof. In still further embodiments, the V_{α} domain encoding polynucleotide further comprises a polynucleotide encoding an α-chain constant domain having at least 90% sequence identity to the amino acid sequence of SEQ ID NO:15 and the polynucleotide encoding the α-chain constant domain is at least 80% identical to, comprises or consists of the polynucleotide sequence of SEQ ID NO:46, the V_{β} domain encoding polynucleotide further comprises a polynucleotide encoding a β-chain constant domain having at least 90% sequence identity to the amino acid sequence of SEQ ID NO:16 or 17 and the polynucleotide encoding the β-chain constant domain is at least 80% identical to, comprises or consists of the polynucleotide sequence of SEQ ID NO:47 or 48, or any combination thereof.

In certain embodiments, any of the aforementioned isolated polynucleotides encoding a binding protein has a V_{α} domain-encoding polynucleotide that is contained in a polynucleotide encoding a TCR α-chain having at least 90% sequence identity to the amino acid sequence of any one of SEQ ID NOS:77-83 and the polynucleotide encoding the TCR α-chain is at least 75% or at least 79% or at least 80% identical to the polynucleotide sequence of any one of SEQ ID NOS:112-118; and a V_{β} domain encoding polynucleotide is contained in a polynucleotide encoding a TCR β-chain having at least 90% sequence identity to the amino acid sequence of any one of SEQ ID NOS:84-97 and the polynucleotide encoding the TCR β-chain is at least 80% identical to the polynucleotide sequence of any one of SEQ ID NOS:119-132.

In particular embodiments, any of the aforementioned isolated polynucleotide embodiments encoding a binding protein have structure from 5′-end to 3′-end of ([V_{β} domain encoding polynucleotide]-[self-cleaving peptide]-[V_{α} domain encoding polynucleotide]). In further embodiments, the encoded V_{β} domain, self-cleaving peptide, and V_{α} domain is at least 90% identical to, comprises or consists of the amino acid sequence of any one of SEQ ID NOS:98-111. In still further embodiments, the polynucleotide encoding the ([V_{β} domain]-[self-cleaving peptide]-[V_{α} domain]) is at least 75% or at least 79% or at least 80% identical to, comprises or consists of the polynucleotide sequence of any one of SEQ ID NOS:133-146.

Certain embodiments include nucleic acid molecules of this disclosure contained in a vector. One of skill in the art can readily ascertain suitable vectors for use with certain embodiments disclosed herein. An exemplary vector may comprise a nucleic acid molecule capable of transporting another nucleic acid molecule to which it has been linked, or which is capable of replication in a host organism. Some examples of vectors include plasmids, viral vectors, cosmids, and others. Some vectors may be capable of autonomous replication in a host cell into which they are introduced (e.g. bacterial vectors having a bacterial origin of replication and episomal mammalian vectors), whereas other vectors may be integrated into the genome of a host cell or promote integration of the polynucleotide insert upon introduction into the host cell and thereby replicate along with the host genome (e.g., lentiviral vector)). Additionally, some vectors are capable of directing the expression of genes to which they are operatively linked (these vectors may be referred to as “expression vectors”). According to related embodiments, it is further understood that, if one or more agents (e.g., polynucleotides encoding binding proteins or fusion proteins specific for 5T4, as described herein) is co-administered to a subject, that each agent may reside in separate or the same vectors, and multiple vectors (each containing a different agent the same agent) may be introduced to a cell or cell population or administered to a subject.

In certain embodiments, polynucleotides encoding binding proteins or fusion proteins specific for 5T4 may be operatively linked to certain elements of a vector. For example, polynucleotide sequences that are needed to effect the expression and processing of coding sequences to which they are ligated may be operatively linked. Expression control sequences may include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest. In certain embodiments, polynucleotides encoding binding proteins of the instant disclosure are contained in an expression vector that is a viral vector, such as a lentiviral vector or a γ-retroviral vector.

In certain embodiments, the vector comprises a plasmid vector or a viral vector (e.g., a vector selected from lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, and spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses. “Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing TCR or CAR transgenes are known in the art and have been previous described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors recently developed for gene therapy uses can also be used with the compositions and methods of this disclosure. Such vectors include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as Sleeping Beauty or other transposon vectors). When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

In particular embodiments, the recombinant expression vector is capable of delivering a polynucleotide to an appropriate host cell, for example, a T cell or an antigen-presenting cell, i.e., a cell that displays a peptide/MHC complex on its cell surface (e.g., a dendritic cell) and lacks CD8. In certain embodiments, the host cell is a hematopoietic progenitor cell or a human immune system cell. For example, the immune system cell can be a CD4⁺ T cell, a CD8⁺ T cell, a CD4⁻ CD8⁻ double negative T cell, a γδ T cell, a natural killer cell, a dendritic cell, or any combination thereof. In certain embodiments, wherein a T cell is the host, the T cell can be naïve, a central memory T cell, an effector memory T cell, or any combination thereof. The recombinant expression vectors may therefore also include, for example, lymphoid tissue-specific transcriptional regulatory elements (TREs), such as a B lymphocyte, T lymphocyte, or dendritic cell specific TREs. Lymphoid tissue specific TREs are known in the art (see, e.g., Thompson et al., Mol. Cell. Biol. 12:1043, 1992); Todd et al., J. Exp. Med. 177:1663, 1993); Penix et al., Exp. Med. 178:1483, 1993).

In addition to vectors, certain embodiments relate to host cells that comprise the vectors that are presently disclosed. One of skill in the art readily understands that many suitable host cells are available in the art. A host cell may include any individual cell or cell culture which may receive a vector or the incorporation of nucleic acids and/or proteins, as well as any progeny cells. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

Also provided are compositions that comprise a modified immune cell as described herein and a pharmaceutically acceptable carrier, diluent, or excipient. Suitable excipients include water, saline, dextrose, glycerol, or the like and combinations thereof. In embodiments, compositions comprising modified immune cells, binding proteins, or fusion proteins as disclosed herein further comprise a suitable infusion media. Suitable infusion media can be any isotonic medium formulation, typically normal saline, Normosol R (Abbott) or Plasma-Lyte A (Baxter), 5% dextrose in water, Ringer's lactate can be utilized. An infusion medium can be supplemented with human serum albumin or other human serum components.

Unit doses comprising an effective amount of a modified immune cell, host cell, or composition are also contemplated.

### Methods of Treatment

In certain aspects, the instant disclosure is directed to methods for treating a hyperproliferative disorder or a condition characterized by 5T4 expression by administering to human subject in need thereof a modified immune cell, composition, or unit dose of the present disclosure. A condition associated with 5T4 expression includes any disorder or condition in which underactivity, over-activity or improper activity of a 5T4 cellular or molecular event is present, and may result from unusually high levels of 5T4 expression (with statistical significance) or inappropriate (i.e., not occurring in healthy cells of the given cell type) expression in afflicted cells (e.g., renal carcinoma cells), relative to normal cells. A subject having such a disorder or condition would benefit from treatment with a composition or method of the presently described embodiments. Some conditions associated with 5T4 expression thus may include acute as well as chronic disorders and diseases, such as those pathological conditions that predispose the subject to a particular disorder.

Some examples of conditions associated with 5T4 expression include hyperproliferative disorders. The presence of a hyperproliferative disorder or malignant condition in a subject refers to the presence of dysplastic cells, cancerous cells, transformed cells, or a combination thereof, in the subject, including, for example neoplastic, tumor, non-contact inhibited, oncogenically transformed cells, or the like (e.g., solid cancers such as renal, gastric, ovarian, and colorectal cancers; hematologic cancers including lymphomas and leukemias, such as from acute lymphoblastic leukemia (ALL), acute myeloid leukemia (AML), chronic myelogenous leukemia (CIVIL), chronic eosinophilic leukemia (CEL), chronic lymphocytic leukemia (CLL), Hodgkin's lymphoma, myelodysplastic syndrome (MDS), non-Hodgkin's lymphoma (NHL), mixed phenotype acute leukemia (MPAL), a central nervous system lymphoma, small lymphocytic lymphoma (SLL), CD37⁺ dendritic cell lymphoma, lymphoplasmacytic lymphoma, splenic marginal zone lymphoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, precursor B-lymphoblastic lymphoma, immunoblastic large cell lymphoma, intravascular large B-cell lymphoma, primary effusion lymphoma, Burkitt's lymphoma, or multiple myeloma (MM), which are known in the art and for which criteria for diagnosis and classification are established (e.g., Hanahan and Weinberg, Cell 144:646, 2011; Hanahan and Weinberg, Cell 100:57, 2000; Cavallo et al., Canc. Immunol. Immunother. 60:319, 2011; Kyrigideis et al., J. Carcinog. 9:3, 2010)). In particular, neoplastic conditions associated with 5T4 expression have been observed in the Ampulla of Vader, bladder, brain, (apendyoma and neuroblastoma), breast, cervix, colon, endometrium, kidney, prostate, lung, esophagus, ovary, pancreas, soft tissue, stomach, testis, thyroid, and trophoblast (Stern and Harrop, Cancer Immunol. Immunother. 66:415, 2016; Southall et al., Br. J. Cancer 61:89, 1990). In addition to activated or proliferating cells, the hyperproliferative disorder may also include an aberration or dysregulation of cell death processes, whether by necrosis or apoptosis. Such aberration of cell death processes may be associated with a variety of conditions, including cancer (including primary, secondary malignancies as well as metastasis), or other conditions.

According to certain embodiments, virtually any type of cancer that is characterized by 5T4 expression may be treated through the use of compositions and methods disclosed herein, including solid cancers (e.g., kidney cancer, renal cell carcinoma, gastric cancer, ovarian cancer, colorectal cancer, and other solid cancers). Furthermore, “cancer” may refer to any accelerated proliferation of cells, including solid tumors, ascites tumors, blood or lymph or other malignancies; connective tissue malignancies; metastatic disease; minimal residual disease following transplantation of organs or stem cells; multi-drug resistant cancers, primary or secondary malignancies, angiogenesis related to malignancy, or other forms of cancer.

In further embodiments, there are provided methods for treating a hyperproliferative disorder, such as a solid cancer is selected from biliary cancer, bladder cancer, bone and soft tissue carcinoma, brain tumor, breast cancer, cervical cancer, colon cancer, colorectal adenocarcinoma, colorectal cancer, desmoid tumor, embryonal cancer, endometrial cancer, esophageal cancer, gastric cancer, gastric adenocarcinoma, glioblastoma multiforme, gynecological tumor, head and neck squamous cell carcinoma, hepatic cancer, lung cancer (including non-small cell lung cancer (NSCLC)), mesothelioma, malignant melanoma, osteosarcoma, ovarian cancer, pancreatic cancer, pancreatic ductal adenocarcinoma, primary astrocytic tumor, primary thyroid cancer, prostate cancer, renal cancer, renal cell carcinoma, rhabdomyosarcoma, skin cancer, soft tissue sarcoma, testicular germ-cell tumor, urothelial cancer, uterine sarcoma, or uterine cancer.

In some embodiments, the hyperproliferative disorder is a hematological malignancy. In further embodiments, the hematological malignancy is selected from acute lymphoblastic leukemia (ALL), acute myeloid leukemia (AML), chronic myelogenous leukemia (CIVIL), chronic eosinophilic leukemia (CEL), chronic lymphocytic leukemia (CLL), Hodgkin's lymphoma, myelodysplastic syndrome (MDS), non-Hodgkin's lymphoma (NHL), mixed phenotype acute leukemia (MPAL), a central nervous system lymphoma, small lymphocytic lymphoma (SLL), CD37+ dendritic cell lymphoma, lymphoplasmacytic lymphoma, splenic marginal zone lymphoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, precursor B-lymphoblastic lymphoma, immunoblastic large cell lymphoma, intravascular large B-cell lymphoma, primary effusion lymphoma, Burkitt's lymphoma, or multiple myeloma (MM).

In certain embodiments of the methods, the binding protein, fusion protein, or modified immune cell is capable of promoting an antigen-specific T cell response against a 5T4 in a class I HLA-restricted manner. In some embodiments, the class I HLA-restricted response is transporter-associated with antigen processing (TAP) independent.

In further embodiments, the antigen-specific T cell response comprises at least one of a CD4⁺ helper T lymphocyte (Th) response and a CD8⁺ cytotoxic T lymphocyte (CTL) response. In some embodiments, the CTL response is directed against a cell having 5T4 expression.

Also contemplated within the presently disclosed embodiments are specific embodiments wherein only one of the above types of disease is included, or where specific conditions may be excluded regardless of whether or not they are characterized by 5T4 expression.

As understood by a person skilled in the medical art, the terms, “treat” and “treatment,” refer to medical management of a disease, disorder, or condition of a subject (i.e., patient, host, who may be a human or non-human animal) (see, e.g., Stedman's Medical Dictionary). In general, an appropriate dose and treatment regimen provide one or more of a modified immune cell, composition, or unit dose, and optionally an adjunctive or combination therapy (e.g., a cytokine, including endogenous, recombinant, or analogs, such as IL-2, aldesleukin, pegylated IL-2 (e.g., NKTR-214; Nektar Therapeutics), IL-7, IL-15, recombinant IL-15 plus receptor chain, IL-21, or any combination thereof), in an amount sufficient to provide therapeutic or prophylactic benefit. Therapeutic or prophylactic benefit resulting from therapeutic treatment or prophylactic or preventative methods include, for example an improved clinical outcome, wherein the object is to prevent or retard or otherwise reduce (e.g., decrease in a statistically significant manner relative to an untreated control) an undesired physiological change or disorder, or to prevent, retard or otherwise reduce the expansion or severity of such a disease or disorder. Beneficial or desired clinical results from treating a subject include abatement, lessening, or alleviation of symptoms that result from or are associated the disease or disorder to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; or overall survival.

“Treatment” can also mean prolonging survival when compared to expected survival if a subject were not receiving treatment. Subjects in need of the methods and compositions described herein include those who already have the disease or disorder, as well as subjects prone to have or at risk of developing the disease or disorder. Subjects in need of prophylactic treatment include subjects in whom the disease, condition, or disorder is to be prevented (i.e., decreasing the likelihood of occurrence or recurrence of the disease or disorder). The clinical benefit provided by the compositions (and preparations comprising the compositions) and methods described herein can be evaluated by design and execution of in vitro assays, preclinical studies, and clinical studies in subjects to whom administration of the compositions is intended to benefit, as described in the examples.

In another aspect, adoptive immunotherapy methods are provided for treating a condition characterized by 5T4 expression in cells of a subject having a hyperproliferative disorder (e.g., a solid cancer or hematological malignancy as disclosed herein), wherein a method comprises administering to the subject an effective amount of a modified immune cell, composition, or unit dose of the present disclosure. In some embodiments, the immune cell is cell is an allogeneic cell, a syngeneic cell, or an autologous cell. In certain embodiments, the immune cell is a human cell. In particular embodiments, the immune cell is a CD4⁺ T cell, a CD8⁺ T cell, a CD4⁻CD8⁻ T cell, a γδ T cell, a natural killer cell, or any combination thereof. In some embodiments, the T cell is a naïve T cell, a central memory T cell, an effector memory T cell, or any combination thereof. Modified immune cells expressing a binding protein or high affinity recombinant TCR specific for 5T4 as described herein may be administered to a subject in a pharmaceutically or physiologically acceptable or suitable excipient or carrier. Pharmaceutically acceptable excipients are biologically compatible vehicles, e.g., physiological saline, which are described in greater detail herein, that are suitable for administration to a human or other non-human mammalian subject. In any of the aforementioned embodiments, a binding protein or high affinity recombinant TCR of this disclosure is specific for human 5T4.

A therapeutically effective dose, in the context of adoptive cell therapy (e.g., an adoptive cell-based immunotherapy), is an amount of host cells (expressing a binding protein specific for human 5T4) used in adoptive transfer that is capable of producing a clinically desirable result (i.e., a sufficient amount to induce or enhance a specific T cell immune response against cells expressing or overexpressing 5T4 (e.g., a cytotoxic T cell response) in a statistically significant manner) in a treated human or non-human mammal. The dosage for any one patient depends upon many factors, including the patient's size, weight, body surface area, age, the particular therapy to be administered, sex, time and route of administration, general health, and other drugs being administered concurrently. Doses will vary, but a preferred dose for administration of a modified immune cell as described herein is about 10⁴ cells/m², about 5×10⁴ cells/m², about 10⁵ cells/m², about 5×10⁵ cells/m², about 10⁶ cells/m², about 5×10⁶ cells/m², about 10⁷ cells/m², about 5×10⁷ cells/m², about 10⁸ cells/m², about 5×10⁸ cells/m², about 10⁹ cells/m², about 5×10⁹ cells/m², about 10¹⁰ cells/m², about 5×10¹⁰ cells/m², or about 10¹¹ cells/m².

In certain embodiments, In certain embodiments, a unit dose comprises (i) a composition comprising at least about 30%, at least about 40%, at least about 50%, at least about 60%, at least about 70%, at least about 80%, at least about 85%, at least about 90%, or at least about 95% modified CD4⁺ T cells, combined with (ii) a composition comprising at least about 30%, at least about 40%, at least about 50%, at least about 60%, at least about 70%, at least about 80%, at least about 85%, at least about 90%, or at least about 95% modified CD8⁺ T cells, in about a 1:1 ratio, wherein the unit dose contains a reduced amount or substantially no naïve T cells (i.e., has less than about 50%, less than about 40%, less than about 30%, less than about 20%, less than about 10%, less than about 5%, or less then about 1% the population of naïve T cells present in a unit dose as compared to a patient sample having a comparable number of PBMCs).

In some embodiments, a unit dose comprises (i) a composition comprising at least about 50% modified CD4⁺ T cells, combined with (ii) a composition comprising at least about 50% modified CD8⁺ T cells, in about a 1:1 ratio, wherein the unit dose contains a reduced amount or substantially no naïve T cells. In further embodiments, a unit dose comprises (i) a composition comprising at least about 60% modified CD4⁺ T cells, combined with (ii) a composition comprising at least about 60% modified CD8⁺ T cells, in about a 1:1 ratio, wherein the unit dose contains a reduced amount or substantially no naïve T cells. In still further embodiments, a unit dose comprises (i) a composition comprising at least about 70% modified CD4⁺ T cells, combined with (ii) a composition comprising at least about 70% modified CD8⁺ T cells, in about a 1:1 ratio, wherein the unit dose contains a reduced amount or substantially no naïve T cells. In some embodiments, a unit dose comprises (i) a composition comprising at least about 80% modified CD4⁺ T cells, combined with (ii) a composition comprising at least about 80% modified CD8⁺ T cells, in about a 1:1 ratio, wherein the unit dose contains a reduced amount or substantially no naïve T cells. In some embodiments, a unit dose comprises (i) a composition comprising at least about 85% modified CD4⁺ T cells, combined with (ii) a composition comprising at least about 85% modified CD8⁺ T cells, in about a 1:1 ratio, wherein the unit dose contains a reduced amount or substantially no naïve T cells. In some embodiments, a unit dose comprises (i) a composition comprising at least about 90% modified CD4⁺ T cells, combined with (ii) a composition comprising at least about 90% modified CD8⁺ T cells, in about a 1:1 ratio, wherein the unit dose contains a reduced amount or substantially no naïve T cells.

In any of the embodiments described herein, a unit dose comprises equal, or approximately equal numbers of engineered CD45RA⁻ CD3⁺ CD8⁺ and engineered CD45RA⁻ CD3⁺ CD4⁺ T_{M} cells.

Certain methods of treatment or prevention contemplated herein include administering a modified immune cell (which may be autologous, allogeneic or syngeneic) comprising a desired nucleic acid molecule as described herein that is stably integrated into the chromosome of the cell. For example, such a cellular composition may be generated ex vivo using autologous, allogeneic or syngeneic immune system cells (e.g., T cells, antigen-presenting cells, natural killer cells) in order to administer a desired, 5T4-targeted T cell composition to a subject as an adoptive immunotherapy. In certain embodiments, a plurality of doses of a modified immune cell as described herein is administered to the subject, which may be administered at intervals between administrations of about two to about four weeks.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity).

An effective amount of a pharmaceutical composition refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutic amount” may be used in reference to treatment, whereas “prophylactically effective amount” may be used to describe administrating an effective amount to a subject that is susceptible or at risk of developing a disease or disease-state (e.g., recurrence or relapse) as a preventative course.

The pharmaceutical compositions described herein may be presented in unit-dose or multi-dose containers, such as sealed ampoules or vials. Such containers may be frozen to preserve the stability of the formulation until infusion into the patient. In certain embodiments, a unit dose comprises an engineered immune cell as described herein at a dose of about 10⁴ cells/m² to about 10¹¹ cells/m². The development of suitable dosing and treatment regimens for using the particular compositions described herein in a variety of treatment regimens, including e.g., parenteral or intravenous administration or formulation.

If the subject composition is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol or polyethylene glycols in mixtures with water. Aqueous solutions or suspensions may further comprise one or more buffering agents, such as sodium acetate, sodium citrate, sodium borate or sodium tartrate. Of course, any material used in preparing any dosage unit formulation should be pharmaceutically pure and substantially non-toxic in the amounts employed. In addition, the active compounds may be incorporated into sustained-release preparation and formulations. Dosage unit form, as used herein, refers to physically discrete units suited as unitary dosages for the subject to be treated; each unit may contain a predetermined quantity of engineered immune cells or active compound calculated to produce the desired effect in association with an appropriate pharmaceutical carrier.

In general, an appropriate dosage and treatment regimen provides the active molecules or cells in an amount sufficient to provide therapeutic or prophylactic benefit. Such a response can be monitored by establishing an improved clinical outcome (e.g., more frequent remissions, complete or partial, or longer disease-free survival) in treated subjects as compared to non-treated subjects. Increases in preexisting immune responses to a tumor protein generally correlate with an improved clinical outcome. Such immune responses may generally be evaluated using standard proliferation, cytotoxicity or cytokine assays, which are routine in the art and may be performed using samples obtained from a subject before and after treatment.

As used herein, administration of a composition or therapy refers to delivering the same to a subject, regardless of the route or mode of delivery. Administration may be effected continuously or intermittently, and parenterally. Administration may be for treating a subject already confirmed as having a recognized condition, disease or disease state, or for treating a subject susceptible to or at risk of developing such a condition, disease or disease state. Co-administration with an adjunctive or combination therapy may include simultaneous and/or sequential delivery of multiple agents in any order and on any dosing schedule (e.g., 5T4-specific recombinant (i.e., engineered) host cells with one or more cytokines (e.g., IL-2, IL-15, aldesleukin, pegylated IL-2 (e.g., NKTR-214), IL-7, IL-15, recombinant IL-15 plus receptor chain, IL-21, or any combination thereof); immunosuppressive therapy (e.g., calcineurin inhibitors, corticosteroids, microtubule inhibitors, low dose of a mycophenolic acid prodrug), or any combination thereof).

In further embodiments, a cytokine is administered sequentially, provided that the subject was administered the recombinant host cell at least three or four times before cytokine administration. In certain embodiments, the cytokine is administered subcutaneously. In still further embodiments, the subject being treated is further receiving immunosuppressive therapy, such as calcineurin inhibitors, corticosteroids, microtubule inhibitors, low dose of a mycophenolic acid prodrug, or any combination thereof. In yet further embodiments, the subject being treated has received a non-myeloablative or a myeloablative hematopoietic cell transplant, wherein the treatment may be administered at least two to at least three months after the non-myeloablative hematopoietic cell transplant. In additional embodiments, the subject had previously received lymphodepleting chemotherapy prior to receiving the modified immune cell. In certain embodiments, a lymphodepleting chemotherapy comprises a conditioning regimen comprising cyclophosphamide, fludarabine, anti-thymocyte globulin, or a combination thereof (e.g., Cy/Flu).

An effective amount of a therapeutic or pharmaceutical composition refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutic amount” may be used in reference to treatment, whereas “prophylactically effective amount” may be used to describe administrating an effective amount to a subject that is susceptible or at risk of developing a disease or disease-state (e.g., recurrence) as a preventative course.

The level of a CTL immune response may be determined by any one of numerous immunological methods described herein and routinely practiced in the art. The level of a CTL immune response may be determined prior to and following administration of any one of the herein described 5T4-specific binding proteins expressed by, for example, a T cell. Cytotoxicity assays for determining CTL activity may be performed using any one of several techniques and methods routinely practiced in the art (see, e.g., Henkart et al., “Cytotoxic T-Lymphocytes” in Fundamental Immunology, Paul (ed.) (2003 Lippincott Williams & Wilkins, Philadelphia, Pa.), pages 1127-50, and references cited therein).

Antigen-specific T cell responses are typically determined by comparisons of observed T cell responses according to any of the herein described T cell functional parameters (e.g., proliferation, cytokine release, CTL activity, altered cell surface marker phenotype, etc.) that may be made between T cells that are exposed to a cognate antigen in an appropriate context (e.g., the antigen used to prime or activate the T cells, when presented by immunocompatible antigen-presenting cells) and T cells from the same source population that are exposed instead to a structurally distinct or irrelevant control antigen. A response to the cognate antigen that is greater, with statistical significance, than the response to the control antigen signifies antigen-specificity.

A biological sample may be obtained from a subject for determining the presence and level of an immune response to a 5T4-derived peptide antigen as described herein. A “biological sample” as used herein may be a blood sample (from which serum or plasma may be prepared), biopsy specimen, body fluids (e.g., lung lavage, ascites, mucosal washings, synovial fluid), bone marrow, lymph nodes, tissue explant, organ culture, or any other tissue or cell preparation from the subject or a biological source. Biological samples may also be obtained from the subject prior to receiving any immunogenic composition, which biological sample is useful as a control for establishing baseline (i.e., pre-immunization) data.

Methods according to this disclosure may, in certain embodiments, further include administering one or more additional agents to treat the disease or disorder in a combination therapy. For example, in certain embodiments, a combination therapy comprises administering modified immune cell or fusion protein of the present disclosure with (concurrently, simultaneously, or sequentially) an immune checkpoint inhibitor. In certain embodiments, a combination therapy comprises administering a modified immune cell or fusion protein of the present disclosure with an agonist of a stimulatory immune checkpoint agent. In certain embodiments, a combination therapy comprises administering a modified immune cell, composition, or unit dose of the present disclosure with a secondary therapy, such as chemotherapeutic agent, a radiation therapy, a surgery, an antibody, or any combination thereof.

As used herein, the term “immune suppression agent” or “immunosuppression agent” refers to one or more cells, proteins, molecules, compounds or complexes providing inhibitory signals to assist in controlling or suppressing an immune response. For example, immune suppression agents include those molecules that partially or totally block immune stimulation; decrease, prevent or delay immune activation; or increase, activate, or up regulate immune suppression. Exemplary immunosuppression agents to target (e.g., with an immune checkpoint inhibitor) include PD-1, PD-L1, PD-L2, LAG3, CTLA4, B7-H3, B7-H4, CD244/2B4, HVEM, BTLA, CD160, TIM3, GALS, KIR, PVR1G (CD112R), PVRL2, adenosine, A2aR, immunosuppressive cytokines (e.g., IL-10, IL-4, IL-IRA, IL-35), IDO, arginase, VISTA, TIGIT, LAIR1, CEACAM-1, CEACAM-3, CEACAM-5, Treg cells, or any combination thereof.

An immune suppression agent inhibitor (also referred to as an immune checkpoint inhibitor) may be a compound, an antibody, an antibody fragment or fusion polypeptide (e.g., Fc fusion, such as CTLA4-Fc or LAG3-Fc), an antisense molecule, a ribozyme or RNAi molecule, or a low molecular weight organic molecule. In any of the embodiments disclosed herein, a method may comprise administering a modified immune cell, composition, or unit dose of the present disclosure with one or more inhibitor of any one of the following immune suppression components, singly or in any combination.

In certain embodiments, modified immune cell, composition, or unit dose of the present disclosure is used in combination with a PD-1 inhibitor, for example a PD-1-specific antibody or binding fragment thereof, such as pidilizumab, nivolumab (Opdivo, formerly MDX-1106), pembrolizumab (Keytruda, formerly MK-3475), MEDI0680 (formerly AMP-514), AMP-224, BMS-936558 or any combination thereof. In further embodiments, a 5T4-specific binding protein of the present disclosure (or an engineered host cell expressing the same) is used in combination with a PD-L1 specific antibody or binding fragment thereof, such as BMS-936559, durvalumab (MEDI4736), atezolizumab (RG7446, MPDL3280A), avelumab (MSB0010718C), or any combination thereof.

In certain embodiments, a modified immune cell, composition, or unit dose of the present disclosure is used in combination with a LAG3 inhibitor, such as LAG525, IMP321, IMP701, 9H12, BMS-986016, or any combination thereof.

In certain embodiments, a modified immune cell, composition, or unit dose of the present disclosure is used in combination with an inhibitor of CTLA4. In particular embodiments, a 5T4-specific binding protein of the present disclosure (or an engineered host cell expressing the same) is used in combination with a CTLA4 specific antibody or binding fragment thereof, such as ipilimumab, tremelimumab, CTLA4-Ig fusion proteins (e.g., abatacept, belatacept), or any combination thereof.

In certain embodiments, a modified immune cell, composition, or unit dose of the present disclosure is used in combination with a B7-H3 specific antibody or binding fragment thereof, such as enoblituzumab (MGA271), 376.96, or both. A B7-H4 antibody binding fragment may be a scFv or fusion protein thereof, as described in, for example, Dangaj et al., Cancer Res. 73:4820, 2013, as well as those described in U.S. Pat. No. 9,574,000 and PCT Patent Publication Nos. WO 2016/40724A1 and WO 2013/025779A1.

In certain embodiments, a modified immune cell, composition, or unit dose of the present disclosure is used in combination with an inhibitor of CD244.

In certain embodiments, a modified immune cell, composition, or unit dose of the present disclosure is used in combination with an inhibitor of BLTA, HVEM, CD160, or any combination thereof. Anti CD-160 antibodies are described in, for example, PCT Publication No. WO 2010/084158.

In certain embodiments, a modified immune cell, composition, or unit dose of the present disclosure is used in combination with an inhibitor of TIM3.

In certain embodiments, a modified immune cell, composition, or unit dose of the present disclosure is used in combination with an inhibitor of Gal9.

In certain embodiments, a modified immune cell, composition, or unit dose of the present disclosure is used in combination with an inhibitor of adenosine signaling, such as a decoy adenosine receptor.

In certain embodiments, a modified immune cell, composition, or unit dose of the present disclosure is used in combination with an inhibitor of A2aR.

In certain embodiments, a modified immune cell, composition, or unit dose of the present disclosure is used in combination with an inhibitor of KIR, such as lirilumab (BMS-986015).

In certain embodiments, a modified immune cell, composition, or unit dose of the present disclosure is used in combination with an inhibitor of an inhibitory cytokine (typically, a cytokine other than TGFβ) or Treg development or activity.

In certain embodiments, a modified immune cell, composition, or unit dose of the present disclosure is used in combination with an IDO inhibitor, such as levo-1-methyl tryptophan, epacadostat (INCB024360; Liu et al., Blood 115:3520-30, 2010), ebselen (Terentis et al., Biochem. 49:591-600, 2010), indoximod, NLG919 (Mautino et al., American Association for Cancer Research 104th Annual Meeting 2013; Apr. 6-10, 2013), 1-methyl-tryptophan (1-MT)-tira-pazamine, or any combination thereof.

In certain embodiments, a modified immune cell, composition, or unit dose of the present disclosure is used in combination with an arginase inhibitor, such as N(omega)-Nitro-L-arginine methyl ester (L-NAME), N-omega-hydroxy-nor-1-arginine (nor-NOHA), L-NOHA, 2(S)-amino-6-boronohexanoic acid (ABH), S-(2-boronoethyl)-L-cysteine (BEC), or any combination thereof.

In certain embodiments, a modified immune cell, composition, or unit dose of the present disclosure is used in combination with an inhibitor of VISTA, such as CA-170 (Curis, Lexington, Mass.).

In certain embodiments, a modified immune cell, composition, or unit dose of the present disclosure is used in combination with an inhibitor of TIGIT such as, for example, COM902 (Compugen, Toronto, Ontario Canada), an inhibitor of CD155, such as, for example, COM701 (Compugen), or both.

In certain embodiments, a modified immune cell, composition, or unit dose of the present disclosure is used in combination with an inhibitor of PVRIG, PVRL2, or both. Anti-PVRIG antibodies are described in, for example, PCT Publication No. WO 2016/134333. Anti-PVRL2 antibodies are described in, for example, PCT Publication No. WO 2017/021526.

In certain embodiments, a modified immune cell, composition, or unit dose of the present disclosure is used in combination with a LAIR1 inhibitor.

In certain embodiments, a modified immune cell, composition, or unit dose of the present disclosure is used in combination with an inhibitor of CEACAM-1, CEACAM-3, CEACAM-5, or any combination thereof.

In certain embodiments, a modified immune cell, composition, or unit dose of the present disclosure is used in combination with an agent that increases the activity (i.e., is an agonist) of a stimulatory immune checkpoint molecule. For example, modified immune cell, composition, or unit dose of the present disclosure can be used in combination with a CD137 (4-1BB) agonist (such as, for example, urelumab), a CD134 (OX-40) agonist (such as, for example, MEDI6469, MEDI6383, or MEDI0562), lenalidomide, pomalidomide, a CD27 agonist (such as, for example, CDX-1127), a CD28 agonist (such as, for example, TGN1412, CD80, or CD86), a CD40 agonist (such as, for example, CP-870,893, rhuCD40L, or SGN-40), a CD122 agonist (such as, for example, IL-2) an agonist of GITR (such as, for example, humanized monoclonal antibodies described in PCT Patent Publication No. WO 2016/054638), an agonist of ICOS (CD278) (such as, for example, GSK3359609, mAb 88.2, JTX-2011, Icos 145-1, Icos 314-8, or any combination thereof). In any of the embodiments disclosed herein, a method may comprise administering a modified immune cell, composition, or unit dose of the present disclosure with one or more agonist of a stimulatory immune checkpoint molecule, including any of the foregoing, singly or in any combination.

In certain embodiments, a combination therapy comprises a modified immune cell, composition, or unit dose of the present disclosure and a secondary therapy comprising one or more of: an antibody or antigen binding-fragment thereof that is specific for a cancer antigen expressed by the non-inflamed solid tumor, a radiation treatment, a surgery, a chemotherapeutic agent, a cytokine, RNAi, or any combination thereof.

In certain embodiments, a combination therapy method comprises administering a modified immune cell, composition, or unit dose of the present disclosure and further administering a radiation treatment or a surgery. Radiation therapy is well-known in the art and includes X-ray therapies, such as gamma-irradiation, and radiopharmaceutical therapies. Surgeries and surgical techniques appropriate to treating a given cancer or non-inflamed solid tumor in a subject are well-known to those of ordinary skill in the art.

In certain embodiments, a combination therapy method comprises administering a modified immune cell, composition, or unit dose of the present disclosure and further administering a chemotherapeutic agent. A chemotherapeutic agent includes, but is not limited to, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor. Illustrative chemotherapeutic agents include, without limitation, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates—busulfan, nitrosoureas (carmustine (BCNU) and analogs, streptozocin), trazenes—dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); chimeric antigen receptors; cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; chromatin disruptors; HDAC inhibitors (corinostate, romidepsin, chidamide, panobinostate, belinostat, calproic acid, mocteinostat, abexinostat, entinostat, SB939, resminostat, givinostat, quisinostat, HBI-8000, KEvetrin, CUIC-101, AR-42, CHR-2845. CHR-3996. 4SC-202, CG200745, ACy-1214, ME-344); glutaminase inhibitors (e.g., CB-839 (CaliThera Biosciences)); demethylating agents (e.g., 5-azacytidine, 5-azadeoxycytidine, and procaine), and combinations thereof, including combinations with therapeutic antibodies and other biological molecules.

Cytokines are increasingly used to manipulate host immune response towards anticancer activity. See, e.g., Floros & Tarhini, Semin. Oncol. 42(4):539-548, 2015. Cytokines useful for promoting immune anticancer or antitumor response include, for example, IFN-α, IL-2, IL-3, IL-4, IL-7 IL-10, IL-12, IL-13, IL-15, IL-16, IL-17, IL-18, IL-21, IL-24, aldesleukin, pegylated IL-2 (e.g., NKTR-214), recombinant IL-15 plus receptor chain, and GM-CSF, singly or in any combination with a modified immune cell, composition, or unit dose of the present disclosure.

Another cancer therapy approach involves reducing expression of oncogenes and other genes needed for growth, maintenance, proliferation, and immune evasion by cancer cells. RNA interference, and in particular the use of microRNAs (miRNAs) and small inhibitory RNAs (siRNAs) provides an approach for knocking down expression of cancer genes (see, e.g., Larsson et al., Cancer Treat. Rev. 16(55):128-135, 2017), which can be used in combination with a modified immune cell, composition, or unit dose of the present disclosure.

In any of the embodiments disclosed herein, a modified immune cell, composition, or unit dose may be administered with a second agent targeting 5T4 in a combination therapy. Accordingly, in certain embodiments, a modified immune cell, composition, or unit dose may be administered prior to, contemporaneous with, or following administration to the subject of one or more of TroVax®, mAb5T4 or an antigen-binding fragment derived therefrom, the “superantigen” ABR-214936, or an antibody-drug conjugate comprising an antibody that specifically binds to 5T4 conjugated to a drug such as, for example, the microtubule inhibitor monomethyl auristatin phenylalanine (MMAF) via a maleimidocaproyl (mc) linker (e.g. PF-06263507).

In any of the embodiments disclosed herein, any of the therapeutic agents (e.g, a modified immune cell, composition, unit dose, an inhibitor of an immune suppression component, an agonist of a stimulatory immune checkpoint molecule, an antitumor lymphocyte, a chemotherapeutic agent, a radiation therapy, a surgery, a cytokine, or an inhibitory RNA) may be administered once or more than once to the subject over the course of a treatment, and, in combinations, may be administered to the subject in any order or any combination. An appropriate dose, suitable duration, and frequency of administration of a therapeutic agent will be determined by such factors as a condition of the patient; size, type, spread, growth, and severity of the tumor or cancer; particular form of the active ingredient; and the method of administration.

### EXAMPLES

### Example 1

### Production and Characterization of High-Affinity 5T4 TCRs

5T4₁₇₋₂₅:HLA-A2 specific clones were isolated from patients with metastatic clear cell renal cell carcinoma (RCC) as described in Tykodi et al., J. Immunother. 35(7):523-533 (2012). Briefly, CD8⁺ T cells from HLA-A2⁺ healthy donors (n=4) or RCC patients (n=2) were stimulated in vitro with an HLA-A2-binding nonamer peptide 5T4₁₇₋₂₅ and screened by flow cytometry with specific tetramers. For the isolation and expansion of high-avidity 5T4₁₇₋₂₅ peptide-specific CD8⁺ effectors from peripheral blood mononuclear cells (PBMC) of HLA-A2⁺ normal donors or RCC patients, dendritic cells to serve as antigen-presenting cells for peptide were generated from peripheral blood monocytes by 2-day cultures as described (Dauer, 2003). CD8⁺ T cells were enriched from PBMC by negative selection using magnetic bead separation per the manufacturer's protocol (Miltenyi Biotec, Auburn, Calif.). CD8⁺ T cells were cultured in T25 flasks to contain approximately 1.0×10⁷ CD8⁺ T cells and 5×10⁶ dendritic cells in CTL media (Warren, 1998). Cultured T cells were contacted with 5T4 peptides at a concentration between 10 and 0.01 mg/mL at 10-fold decrements, or with no added peptide. After 24 hours, IL-7 (10 ng/mL) and IL-12 (10 ng/mL) (both from R&D Systems, Minneapolis, Minn.) were added to the cultures. Responder T cells were restimulated with peptide at 10- to 12-day intervals using monocyte antigen presenting cells enriched from PBMC by plastic adherence in AIM-V media (Invitrogen)/1% human serum for 1 hour at 37° C. One day after the second and third peptide stimulation, IL-2 (Prometheus, San Diego, Calif.) was added at 25 IU/mL. Cultured T cells were evaluated by flow cytometry 10-12 days after the second or third peptide stimulation by immunostaining with a fluorochrome-conjugated HLA-A2/5T4(17-25) specific tetramer (TET). CD8⁺/TET⁺ cells were flow sorted from T cell lines cultured with a limiting dose of stimulating 5T4(17-25) peptide (0.1 or 1.0 μg/mL), or from cultures stimulated with the highest dose of 5T4(17-25) peptide (10 μg/mL). Cloning of CD8⁺/TET⁺ cells was performed by limiting dilution in 96-well microtiter cultures. Wells contained 1×10⁵ irradiated (35 Gy) PBMC and 1×10⁴ irradiated (70 Gy) EBV-transformed lymphoblastoid cell line (LCL) feeder cells in 200 mL CTL culture media containing 30 ng/mL OKT3 (Centocor Ortho Biotech Inc., Raritan, N.J.) and 50 IU/mL IL-2. Responder T cells were added at 200-300 per 96-well plate. Positive wells were identified by microscopy after 10-12 days.

Approximately one quarter of the growing clones per each T cell culture was harvested in total and screened for lysis of T2 target cells loaded with serial 10× dilutions of specific peptide (1.0, 0.1, 0.01 nM) or T2 without specific peptide to identify a limiting concentration of detectable peptide. The following day, the remaining positive cloning wells were harvested (½ well volume) and tested for lytic activity versus T2 targets pulsed with the empirically defined limiting peptide concentration or no peptide. Clones with the highest lytic potency for peptide-loaded T2 targets were then expanded and maintained in culture as described (Brodie, 1999; Tykodi et. al. J Immunother. 35(7): 523 (2012); Dauer et al. J Immunol. 170(8):4069 (2003); Warren et al. Blood. 91:2197 (1998); Brodie et al. Nat Med. 5:34 (1999)). HEK293T cell line was obtained from ATCC (Rockville, Md.) and cultured in RPMI media with 10% fetal bovine serum (Gibco, Waltham, Wash.), 1% of L-glutamine (Gibco) and 1% penicillin and streptomycin (LCL media). Lentivirus packaging plasmids (pRSV-REV, pMD2-G and pMDLg/pRRE) and the TCR encoding-construct backbone (pRRLSIN) were provided by Dr. Phillip Greenberg (Fred Hutchinson Cancer Research Institute, Seattle, Wash.). 5T4 protein expression and HLA-A type of tumor lines were listed as follows (if not marked, tumor lines were from Dr. B. Van den Eynde, Ludwig Institute for Cancer Research, Brussels, Belgium): TREP (RCC), HLA-A2⁺/5T4⁺; SW480 (colorectal tumor line, from ATCC): HLA-A2⁺/5T4⁺, MDA-231 (Breast Carcinoma, from ATCC), HLA-A2⁺/5T4⁺; BT20 (Breast Carcinoma, from ATCC), HLA-A2-5T4⁺; DOBSKI (RCC), HLA-A2⁺/5T4⁺; A498 (RCC), HLA-A2⁺/5T4⁺; AMM (RCC, control of A498), HLA-A2⁺/5T4⁺; CAJE-RCC (RCC) HLA-A275T4⁺; CAJE-LCL (LCL, control of CAJE RCC), HLA-A2⁺/5T4⁻. Tumor lines were maintained in CTL media with the following composition: RPMI media with 10% human serum (Fred Hutchinson Cancer Research Institute, Seattle, Wash.), 1% of L-glutamine (Gibco), 1% penicillin and streptomycin (Gibco), and 1% sodium pyruvate (Invitrogen, Carlsbad, Calif.) (Tykodi et al. (2012)).

### TCR Sequencing

Genomic DNA was isolated using QIAamp blood mini kits (Qiagen, Hilden, Germany) from 19 CD8⁺ T cell clones specific for the 5T4ₚ₁₇ epitope presented by HLA-A2. High-throughput, T-cell receptor β chain (TRB) sequencing using the ImmnoSeq assay (Adaptive Biotechnologies, Seattle, Wash.) was performed on all samples at survey level resolution (see Sherwood et al. Sci. Transl. Med. 3(90):90ra61 (2011). The final library pool for each set of PBMC samples was subsequently sequenced using a v3 150 cycle on the Illumina MiSeq platform in the Genomics Core Facility at the Fred Hutchinson Cancer Research Center. Repertoire analyses were conducted using the LymphoSeq R package (created by D. G. Coffey) and implemented in the R statistical computing environment (http://bioconductor.org/packages/LymphoSeq).

Single-cell sequencing was conducted based on the protocol described in Han et al. Nature Biotech. (32):684-692 (2014). T cells from 5T4-specific clones were stained with monoclonal antibodies (mAbs) APC-Cy7-labeled anti-CD3 (clone SK7; BD Biosciences, San Jose, Calif.), FITC-labeled anti-CD8, (clone RPA-T8; BD Biosciences) and the DAPI viability marker, and CD8⁺CD3⁺ DAPI⁻ cells were sorted into one cell per well of a 96-well plate. Targeted CDR3 region mRNA transcripts (TRA and TRB) were then reverse-transcribed into cDNA library using one-step RT PCR kit (Qiagen, Hilden, Germany). A library was constructed using the 96-well barcodes and PCR protocol described in Han et al. (Nature Biotech. (2014), supra). The end PCR products were approximately 380 bp in length, with an Illumina® specific adaptor attached to both ends of the product. Library products were pooled together and purified using Agencourt AMPure XP® beads (Beckman Coulter, Brea, Calif.). Library concentration and purity were quantified using a Nanodrop® spectrophotometer. The library was sequenced (500 cycles) using the MiSeq platform (Illumina, San Diego, Calif.) with paired end v2 kit. The MiSeq library was de-multiplexed according to well barcodes using a Unix script, and CDR3 regions with associated V (D) J region information were extracted by applying MiXCR alignment package to the de-multiplexed reads (Bolotin et al., Nat. Methods 12(5):380-1 (2015). Net charges of CDR regions were computed by the R package “Peptides” (Rice et al., Trends Genet. 16(6):276-7 (2000)).

### CDR3 Sequences of 5T4-Specific TCRs

To examine TCR-β gene usage by 5T4 p17-specific clones, CDR3 regions from the TCR-β gene (TRB-CDR3) expressed by nineteen 5T4ₚ₁₇₋₂₅-specific CD8⁺ T cell clones were analyzed. Seven unique TRB-CDR3 sequences were identified, with only one unique sequence corresponding to each of the seven originating T cell lines.

The identification of only a single TRB-CDR3 sequence from each independent T cell line indicates that the precursor frequency for the originating T cell clone could be as low as one cell out of the initial input 10⁷ CD8⁺ T cells. DNA template was generated from flow-sorted CD8⁺ T cells purified from available donor samples that included the initiating leukapheresis product for T cell lines from donors three donors (two healthy, one patient with metastatic clear cell RCC) and two additional PBMC samples and a tumor-infiltrating lymphocyte sample also from the patient. Sequencing of TRB-CDR3 from these heterogenous T cell samples did not detect the TRB-CDR3 sequences associated with corresponding 5T4p17-specific CD8⁺ T cell clones isolated from each of these donors. These analyses suggest an upper boundary for precursor frequencies for these clones at less than 1 in 1 to 1.5×10⁵ CD8+ T cells.

To identify the TCR-α gene CDR3 region (TRA-CDR3) pairing with each of the unique TRB-CDR3, targeted single-cell RNA-Seq of the CDR3 regions of TRA and TRB genes was then performed on representative T cell clones for each unique TRB-CDR3 sequence. This revealed seven unique single TRA-CDR3 sequences that each paired with one of the seven TRB-CDR3s. Single cell RNA-Seq analysis of clone17-9B5 confirmed both the nonproductive and productive rearrangement of TRB within each cell.

Analysis of CDR3 sequences revealed a high degree of homology. For example, the TRA-CDR3 region of clone6-5G8 and clone17-9B5 differed by only one amino acid at position 106 (serine of clone6-5G8, glycine of clone17-9B5), while clone3-6C3 and clone6-5G8 differed only at position 105 (serine of clone3-6C3, alanine of clone6-5G8, FIG. 1B). Sequence logos are shown in FIGS. 1C-1F. Grouping the aligned amino acids by IMGT classification revealed common motifs among the several TCRs.

### V(D)J Gene Usage of 5T4-Specific TCRs

Based on the CDR3 sequences, which span the 3′ end of the V gene segment to the 5′ beginning of the J gene segment, V(D)J gene usage of the seven TRAB pairs was obtained using IMGT V-quest (Lefranc, Nucleic Acids Res. 3/(1):307-10 (2003); Brochet et al., Nucleic Acids Res. 36. (Web Server issue):W503-8. This analysis revealed common gene segment usage among subsets of the seven TCR sequences. For example, TRAV38-2 and TRAJ45 were both shared by 4 of 7 TCRs, and TRBV6-3 and TRBJ2-7 were shared by 3 and 2 of 7 TCRs, respectively. Three TCRs used TRAV38-2, TRAJ45, and TRBV6-3. Two TCRs used TRAV38-2 and TRBJ 2-7 (FIG. 1G).

### CDR Net Charges of 5T4-Specific TCRs

Net charges of the 5T4-specific TCR CDRs were calculated using the R package “Peptides” (Rice et al., Trends Genet. 16(6):276-277 (2000)) and are shown in Tables 1 and 2, and represented graphically in FIG. 1H. CDR3 regions were negatively charged. This supports a direct interaction of negatively charged CDR3s and the positively charged 5T4p17 epitope (RLARLALVL (SEQ ID NO:64), with two positively charged arginine residues).

In general, the amino acid sequences of CDR1 and CDR2 regions had negative (slightly below 0 to down to about −2.2) charges. TCRs have been proposed to use a conserved mechanism to bind to HLA-A2 despite lack of consensus sequence motifs in CDR1/CDR2 regions. For example, two unique positive-charged residues (R65 and K66) on the HLA-A2 a1-helix are thought to be key elements to interact with negatively charged residues (Asp and Glu) found in CDR1/CDR2 domains from TCRs specific for HLA-A2 (see Blevins et al., PNAS 113(9):E1276-85 (2016)). V(D)J gene segment usage for HLA-A2 specific tumor antigen recognition is thought to be biased (see Borrman et al., Proteins 85(5):908-919 (2017)). Although the most frequently used V gene segments in the 5T4ₚ₁₇-specific T cell clones (TRAV38 and TRBV6-3) are not among the most common HLA-A2 biased Vα and Vβ genes, the CDR1 amino acid sequences of these two V gene segments are negatively charged at −2 and −0.9, respectively, consistent with preferential binding with HLA-A2 (Table 2 and FIG. 1H).

### Example 2

### Engineered T Cells Expressing Recombinant High-Affinity 5T4 TCRs

### Cloning Full-Length TRA and TRB Sequences

Reference open-reading-frames for the V- and C-gene segments of TRA and TRB were obtained from the International Immunogenetics Information System (Scaveiner & LeFranc, Exp. Clin. Immunogenet. 17(2):83-96 (2000); Folch & LeFranc, Exp. Clin. Immunogenet. 17(1):42-54 (2000)). Vα and Vβ fragments with corresponding CDR3 sequences were assembled for each clone with the following overhang sequences attached to both ends to ensure Gibson assembly:

Vα 5′:
(SEQ ID NO: 147)
AGGAGACGTGGAAGAAAACCCCGGTCCC,
Vα 3′:
(SEQ ID NO: 148)
ACATCCAGAACCCCGACCCTGCAGTGTACCAGCTGCGGGAC,
Vβ 5′:
(SEQ ID NO: 149)
TCCCCGAGCTCAATAAAAGAGCCCACAACCCCTCACTCGGCGCGCCGGC
CACC,
Vβ 3′:
(SEQ ID NO: 150)
GTGTTCCCCCCAGAGGTGGCCGTGTTCGAG.

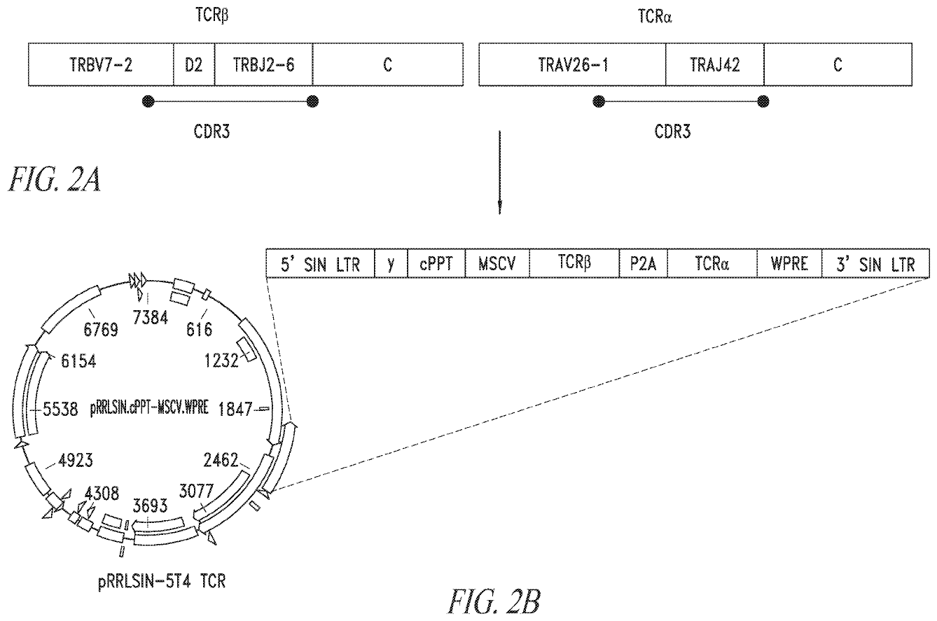

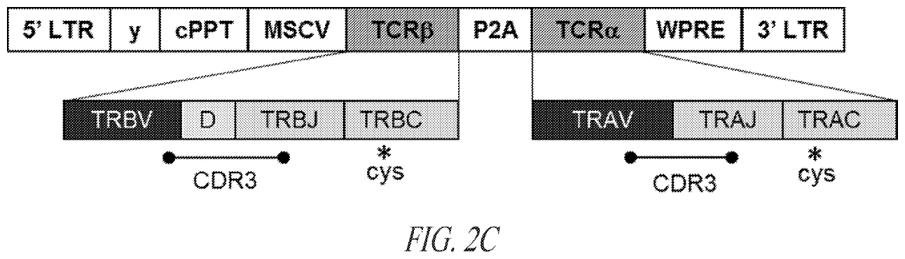

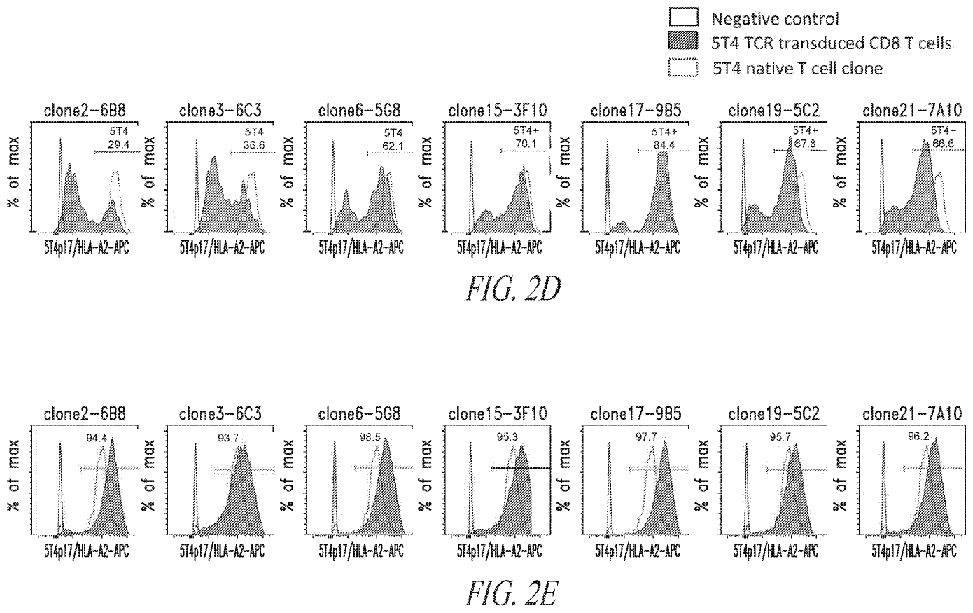

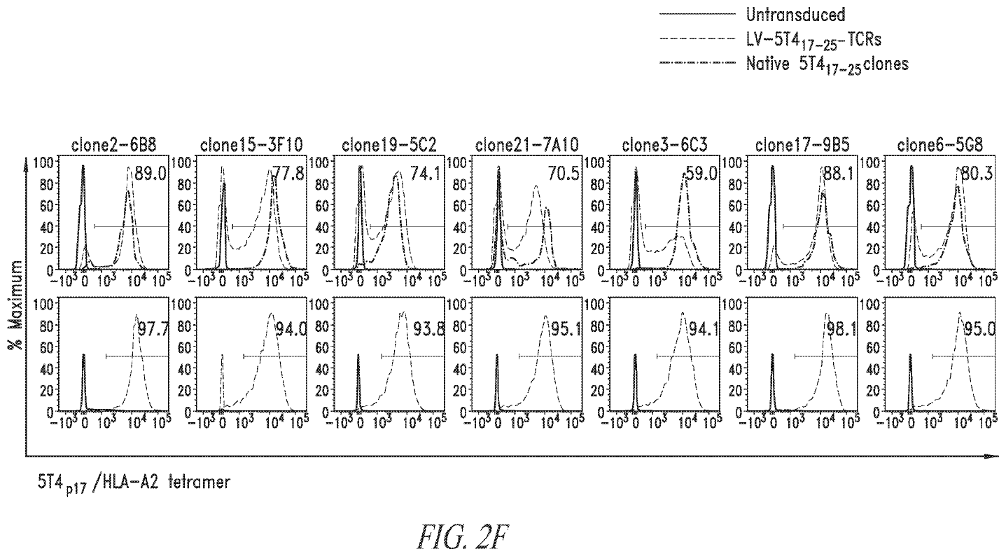

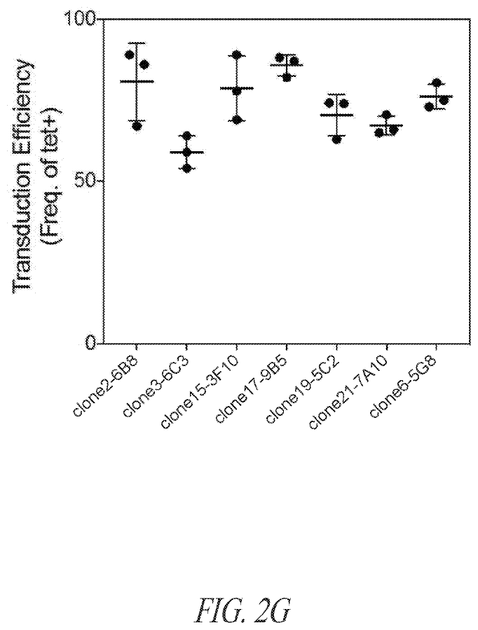

Fragments were synthesized and codon-optimized through GeneArt Strings DNA Fragments and Libraries service (Invitrogen, Carlsbad, Calif.) to improve expression. Constant regions were designed to incorporate cysteine residues at amino acid positions 48 (tyrosine to cysteine) and 57 (serine to cysteine) on TRAC and TRBC, respectively (as in Kuball et al., Blood 2007), to promote crosslinking between the α and β chains when expressed and to ensure that pairing of lentiviral-encoded TCR protein chains occurred preferentially between the 5T4₁₇₋₂₅-specific α and β chains. The stop codon of TRBC was deleted and a sequence encoding a self-cleaving P2A sequence (SEQ ID NO:67) was incorporated between TRB and TRA to ensure equal expression of the 5T4₁₇₋₂₅-specific TCR α and β chains on CD8⁺ T cells. The lentivirus packaging plasmids (pRSV-REV, pMD2-G and pMDLg/pRRE) and TCR encoding backbone plasmid (pRRLSIN) were a gift of Dr. Philip Greenberg (Seattle, Wash.). TCRs were then cloned into lentiviral vectors (pRRLSIN, with a murine stem cell virus (MSCV) promoter included upstream of the TRB and TRA coding regions to selectively drive robust expression of the 5T4₁₇₋₂₅-specific TCR in hematopoietic cells. A Woodchuck posttranscriptional regulatory element (WPRE) and a central polypurine tract (cPPT) element were also included at the 3′ and 5′ ends, respectively, of the TCR coding region to increase transduction efficiency and transgene expression levels (FIGS. 2A-2C). Full-length TCRs were assembled using a Gibson Assembly® ultra kit (SGI-DNA, La Jolla, Calif.). Cloning procedure followed the manufacturer's protocol. Constructs were then transformed into One Shot® TOP10 Chemically Competent E. coli (Invitrogen), and plasmid DNA was extracted using an endotoxin-free Maxi Prep DNA isolation kit (Qiagen). Schematics of exemplary TCR constructs and an expression vector containing the construct are illustrated in FIGS. 2B and 2C.

### Disruption of Endogenous TCR Expression and Inhibitory Loci

Disruption of endogenous TCR in destination cells can inhibit dual specificity and cross-reactivity of engineered T cells after activation, and can prevent mispairing of introduced TCR chains with the wild-type TCR chains, which could result in novel specificities. In addition, due to lack of competition with the endogenous receptor, disrupting endogenous TCR can augment functional avidity of exogenous TCR by increasing cell surface expression. To ensure that immune responses against the 5T4 antigen were specific and due to the activity of the cloned high-affinity 5T4-specific TCRs, endogenous TCR expression in destination CD8⁺ T cells was disrupted prior to transduction with the cloned TCRs. Briefly, a lentivirus expressing CRISPR/Cas9 and a guide RNA targeting the constant region of the endogenous TCRα was constructed. The following primers (Torikai et al., Blood (2016)) were used:

sgRNA Forward Oligo: TRAC_sgRNA_pLenti_F1
(CACCGGAGAATCAAAATCGGTGAAT; SEQ ID NO: 151);
sgRNA Reverse Oligo: TRAC_sgRNA_pLenti_R1
(AAACATTCACCGATTTTGATTCTCC; SEQ ID NO: 152).

TRAC-targeted sgRNA were then cloned into the pLentiCRISPRv2 construct.

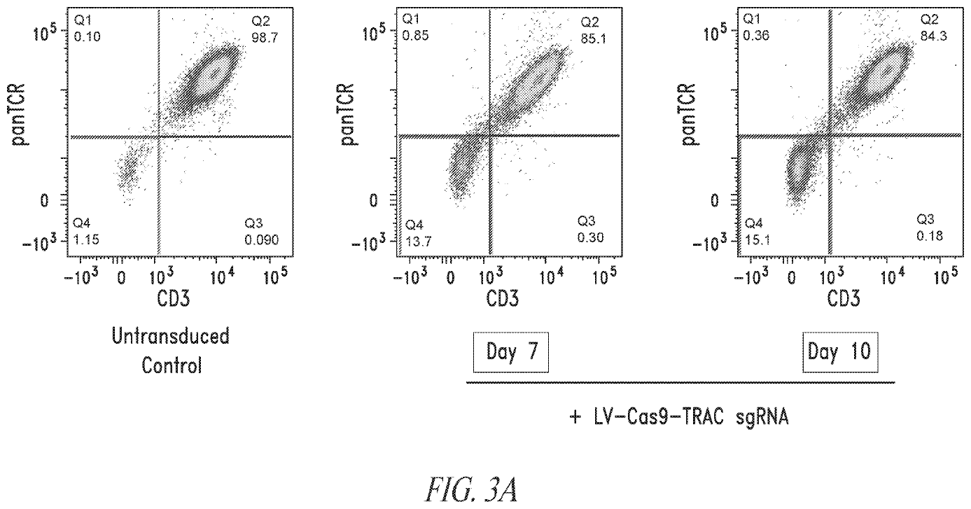

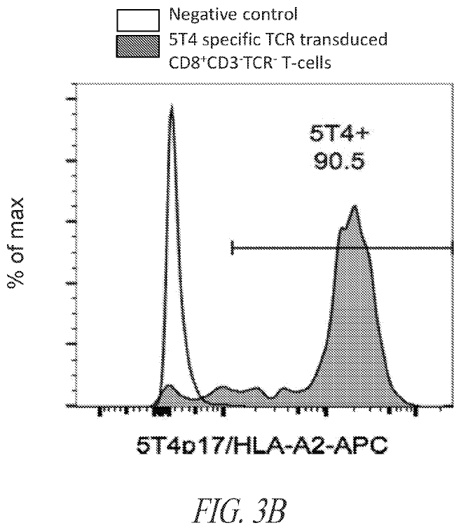

CD8⁺ T-cells were transduced with the lentivirus. Endogenous TCR knockout cells were sorted by panTCR-CD3⁻ gate of T-cell culture (FIG. 3A), and transduced with 5T4₁₇₋₂₅ specific TCR encoding lentivirus. 5T4ₚ₁₇/HLA-A2 tetramer-positive T cells were sorted and expanded (FIG. 3B).

Additional sgRNAs targeting inhibitory loci are generated and cloned into the pLentiCRISPRv2 vector.

For disrupting PD-1, the following sgRNA primers are used: sgRNA Forward Oligo: PD1_sgRNA_F1 (CACCGCAGTTGTGTGACACGGAAG; SEQ ID NO:153); sgRNA Reverse Oligo: PD1_sgRNA_R1 (AAACCTTCCGTGTCACACAACTGC; SEQ ID NO:154).

For disrupting CTLA4, the following sgRNA primers are used: sgRNA Forward Oligo: CTLA4_sgRNA_F1 (CACCGGCAAAGGTGAGTGAGACTTT; SEQ ID NO:155); sgRNA Reverse Oligo: CTLA4_sgRNA_R1 (AAACAAAGTCTCACTCACCTTTGCC; SEQ ID NO:156).

For disrupting LAG3, the following sgRNA primers are used: sgRNA Forward Oligo: LAG3_sgRNA_F1 (CACCGgtttctgcagccgctttggg; SEQ ID NO:157); sgRNA Reverse Oligo: LAG3_sgRNA_R2 (AAACcccaaagcggctgcagaaacC; SEQ ID NO:158).

### Lentiviral Packaging and T-Cell Transduction

The 5T4ₚ₁₇₋₂₅:HLA-A2 specific TCR encoding lentivirus vector was co-transfected with lentivirus packaging plasmids pRRSIN-TCR (1.5 μg), pRSV-REV (1 μg), pMD2-G (0.5 μg) and pMDLg/pRRE (1 μg) into Lenti-X HEK293T packaging cells (Clontech Laboratories, Mountain View, Calif.) were seeded at 60% confluency. using Effectene transfection reagent (Qiagen) following the manufacturer's protocol. Media was changed the following morning; 48 hours post-transfection, lentivirus-containing LCL supernatants were harvested each morning for two days and filtered through 0.45 μm syringe filters. Viral supernatants were then concentrated using Lenti-X viral concentrator (Clontech Laboratories), aliquoted and applied to T cells for transduction or stored at −80° C.

Healthy CD8⁺ T-cells were isolated from donor PBMCs using a human CD8⁺ T cell isolation kit (Miltenyi Biotec, Bergisch Gladbach, Germany) and activated with CD3/CD28 Dynabeads® human T-Cell expander (Thermo Fisher Scientific, Waltham, Mass.) for 4 hours. TCR-encoding lentivirus was applied with 6 μg/ml polybrene (Sigma-Aldrich, St. Louis, Mo.) to CD8⁺ T cells, and the cells were centrifuged at 1455×g for 90 minutes at 35° C. The next day, the same transduction procedure was repeated with virus and fresh media. T cell cultures were then maintained in human IL-2 (PeproTech, Rocky Hill, N.J.) containing CTL media for 7 days before assessing 5T4ₚ₁₇₋₂₅:HLA-A2 specific TCR expression by tetramer immunostaining and flow cytometry analysis.

### Synthetic Peptide and HLA Tetramer Reagents

Synthetic peptides corresponding to 5T4ₚ₁₇ RLARLALVL (SEQ ID NO:64), and alanine-substituted variant sequences ALARLALVL (R1A; SEQ ID NO:168), RLAALALVL (R4A; SEQ ID NO:169), RLARAALVL (L5A; SEQ ID NO:170), RLARLAAVL (L7A; SEQ ID NO:171), RLARLALAL (V8A; SEQ ID NO:172) at a purity >90% (Genscript, Piscataway, N.J.) were dissolved in 100% dimethyl sulfoxide (Invitrogen) and stored at 4° C. Peptide-class I MHC-A2 monomers were folded and purified (Immune Monitoring Lab, Fred Hutchinson Cancer Research Institute), and APC-conjugated tetramers were made.

### Flow Cytometry

On day 7 post-transduction, T cell cultures were stained with DAPI, a 1:20 dilution of APC-Cy7-labeled anti-human CD3 mAb (clone SK7; BD Biosciences, San Jose, Calif.), 1:20 dilution of FITC-labeled anti-human CD8 mAb (clone RPA-T8; BD Biosciences), and 10 μg/mL of 5T4ₚ₁₇₋₂₅/HLA-A2-APC tetramer (TET) and analyzed by by flow cytometry (BD FACSymphony™, BD Biosciences).

Viable 5T4ₚ₁₇₋₂₅/HLA-A2-APC⁺CD8⁺CD3⁺DAPI⁻ T cells were flow sorted (BD FACSAria™; BD Biosciences) and expanded by 30 ng/ml OKT-3 antibody (monoclonal anti-human CD3, Ortho Biotech Products, through UW Pharmacy, Seattle, Wash.), as well as irradiated EBV-transformed B cells (TM-LCL, 70 Gy) and irradiated PBMC (35 Gy) as feeder cells in 50 U/ml IL-2 and 5 ng/mL IL-15 (PeproTech)-containing CTL media for 12 days. 5T4₁₇₋₂₅-specific TCR expression was confirmed by sorting APC⁺CD8⁺CD3⁺DAPI⁻ T cells post-transduction. Results are shown in FIGS. 2D-2F. These data show that the engineered 5T4-specific TCRs are robustly expressed by transduced CD8⁺ T cells. Moreover, expression of the engineered TCRs by transduced cells was generally more robust than 5T4-TCR expression by native T cell source clones.

### Example 3

### Antigen-Specific Activity of Recombinant CD8⁺5 T4-TCR⁺ T Cells

### Target Cell Lines

Maintenance for T2, BB65 LCL, RCC lines A498, BB65, LB1828, DOBSKI and SST548, was performed as described in Tykodi et al. J. Immunother. 35(7):523-33 (2012). The breast cancer lines MDA-231 and BT-20 and the colorectal tumor line SW480 were obtained from ATCC (Manassas, Va.).

### Cytotoxicity and ELISA Assays

For some experiments, T2 and LCL targets were infected with wild-type MVA or MVA-5T4 (Oxford Biomedica, Oxford, UK) at a 10:1 multiplicity of infection in serum-free RPMI media at 37° C. for 1 hour. Media serum concentration was then adjusted to 10% FCS. Chromium-release cytotoxicity assays with LCL and tumor target lines were performed as described in Tykodi et al. Clin Cancer Res. 10(23):7799-811 (2004).

To confirm 5T4 expression on target cell surface after MVA-5T4 infection, cells were stained with a 5T4-specific mAb (clone 524744; R&D systems, Minneapolis, Minn.) at 1 ug/ml followed by a 1:50 dilution of a secondary PE-labeled anti-mouse IgG1 mAb, (clone A85-1; BD Biosciences) for analysis by flow cytometry. Supernatants were harvested after 16 hours of T cell-target cell co-culture and assayed for interferon-γ (IFN-γ) or tumor necrosis factor-α (TNF-α) content by enzyme-linked immunosorbent assays (ELISA) according to the manufacturer's protocol (BosterBio, Pleasanton, Calif.).

### HLA A2 Stabilization Assay

Aliquots of 2×10⁵ T2 cells were cultured in serum-free RPMI media supplemented with human β₂-microglobulin at 5 μg/mL plus the test peptide. After overnight incubation at 37° C., cells were stained with an APC-labeled HLA-A2-specific mAb (clone BB7.2, BD Biosciences) at dilution of 1:50 plus DAPI and analyzed by flow cytometry.

Target cells (T2 cells and different tumor cell-lines) were counted and transferred in a 24-well plate per condition in LCL media. 5T4₁₇₋₂₅ peptide was added in assigned wells, and cells were labeled with 0.1 mCi ⁵¹Cr and incubated at 37° C. overnight. The next day, target cells were washed twice with 10 mL LCL media and aliquoted into wells of a round bottom 96-well plate. Effector T cells expressing 5T4ₚ₁₇₋₂₅-specific TCRs were added at a 10 to 1 effector to target ratio (10:1 E:T) and mixed well. Detergent was used as positive control (complete lysis) and LCL media as a negative control (spontaneous release). Cultures were spun for 5 min at 1,200 rpm and incubated at 37° C. for 4 hours. Supernatants were harvested, transferred to a 96-well Lumi-plate and dried overnight. The next day, plates were read using TopCount software. Analysis of cytotoxicity was done by taking the average of triplicate wells and calculating the specific lysis and spontaneous lysis. Specific lysis=(sample−media)/(detergent−media). After 16 hours of co-culture with effector T cells expressing 5T4ₚ₁₇₋₂₅-specific TCRs and target cells, supernatants were harvested and assayed for interferon (IFN)-γ by enzyme-linked immunosorbent assay (ELISA) (Endogen, Rockford, Ill.).

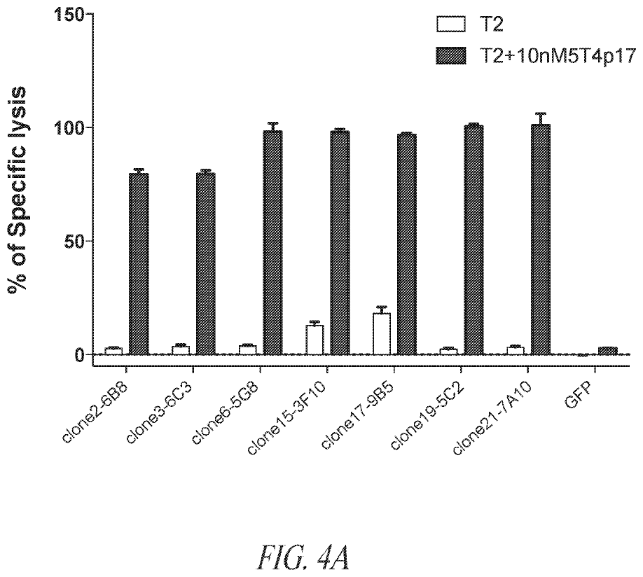

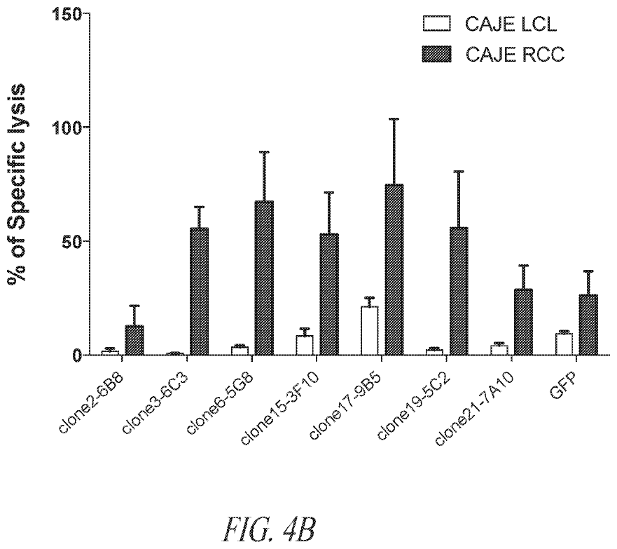

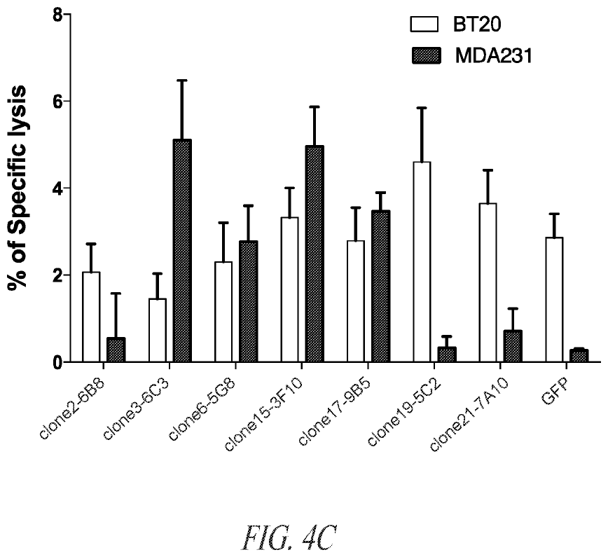

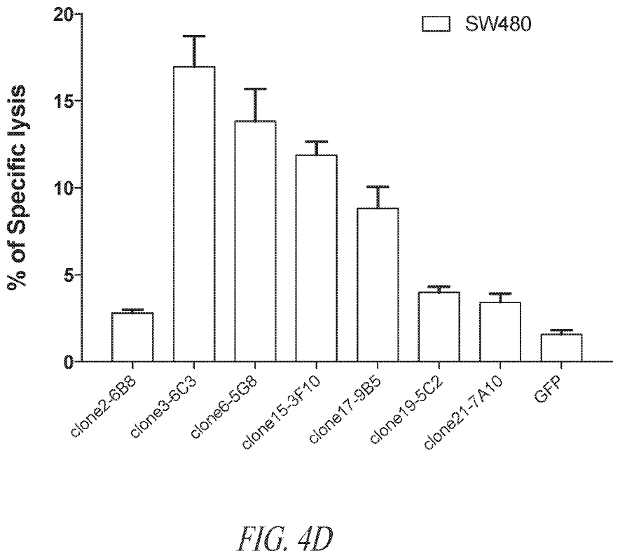

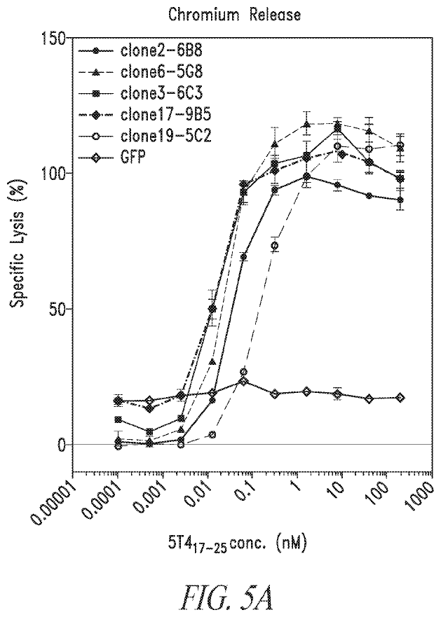

Results from a first set of cytotoxicity experiments are provided in FIGS. 4A-4D. In these experiments, lentivirus transduced CD8⁺ T cells expressing 5T4₁₇₋₂₅ TCRs from 4 donors were co-cultured with T2 cells (without or with the addition of 5T4₁₇₋₂₅ peptide at 10 nM) (4A), RCC and LCL CAJE target cells (4B), breast carcinoma tumor cells (4C), or colorectal tumor cells (4D) in 4-hour cytotoxicity assays with a 10:1 E:T. All target cells for cytotoxicity assays were 5T4+ and HLA-A2⁺ except for the colorectal cell line BT-20, which is HLA-A2⁻ (see FIG. 4C). These data show that 5T4⁺ HLA-A2-expressing tumor cell lines are targets for 5T4₁₇₋₂₅-specific TCR-expressing T cells.

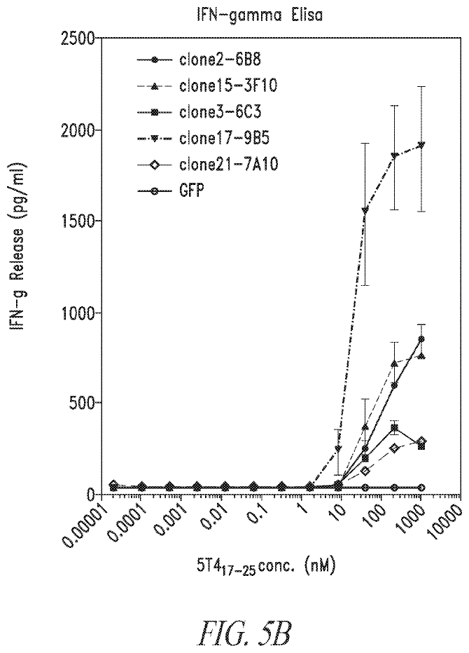

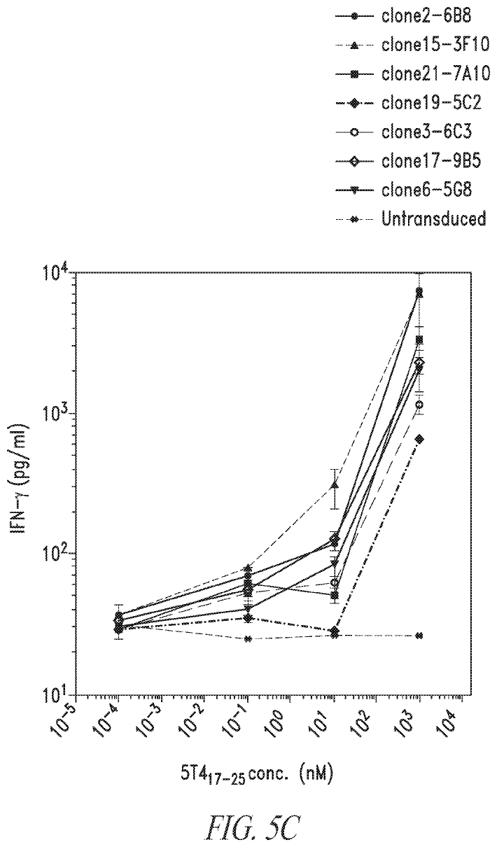

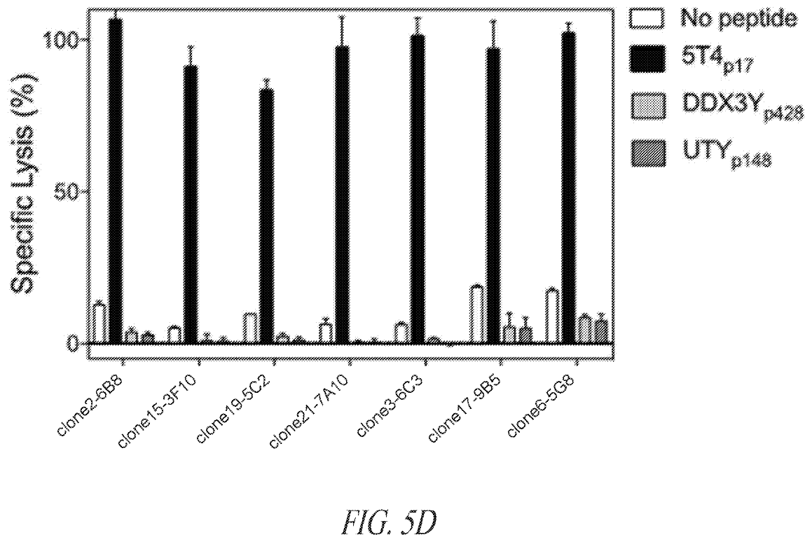

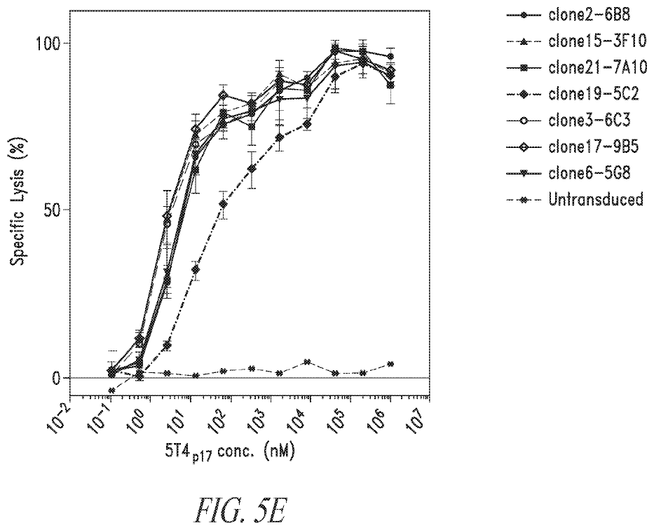

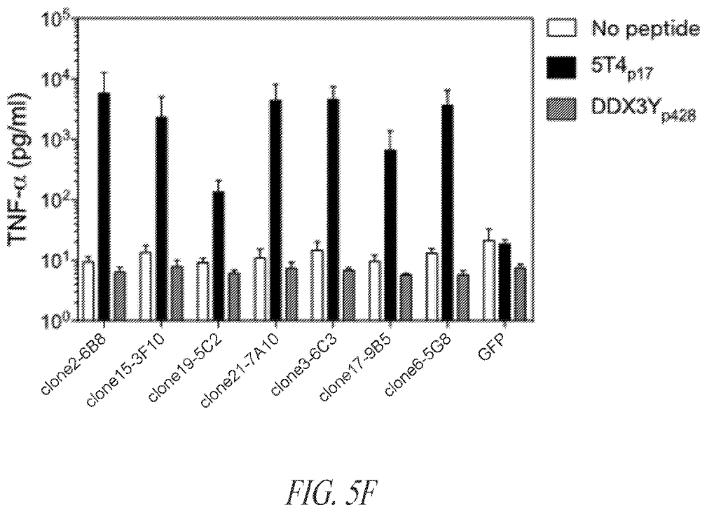

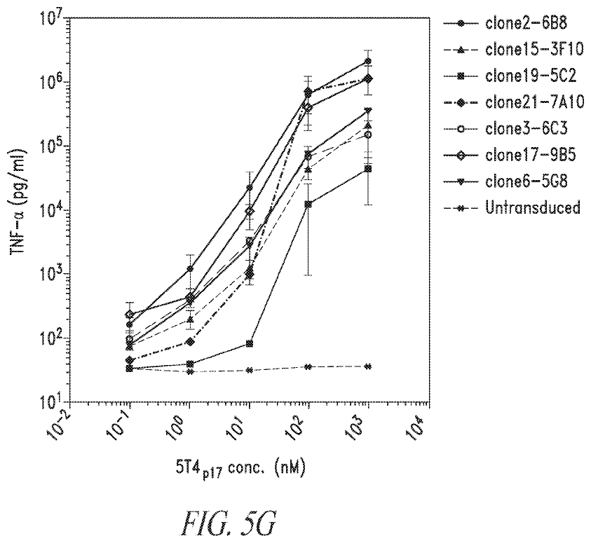

In a second set of experiments, transduced CD8⁺ T cells expressing 5T4₁₇₋₂₅-specific TCRs were co-cultured with T2 target cells pulsed with varying concentrations of the antigen-peptide (FIGS. 5A-5C, 5E, and 5G) or with control HLA-A2-specific peptide (DDX3Y₄₂₈₋₄₃₆ (FLLDILGAT; SEQ ID NO:166) and UTY₁₄₈₋₁₅₆ KAFQDVLYV; SEQ ID NO:167) (FIGS. 5D and 5F). As illustrated in FIG. 5A, ⁵¹Cr release after a 4-hour incubation showed concentration-dependent specific lysis, with all clones exhibiting similar cytotoxic activity and showing above 50% lysis at 1-10 nM peptide (GFP control). As shown in FIGS. 5B and 5C, IFN-γ release at 16 hours was also concentration-dependent, with measurable release at 1-10 nM peptide and above.

As shown in FIGS. 5D and 5E, TCR-transduced T cells exhibited potent cytolytic activity for T2 targets pulsed with 10 nM 5T4p17 peptide without cross-reactivity for control HLA-A2 binding peptide. Transduced T cells also selectively produced TNFα in response to 5T4p17 peptide (FIGS. 5F and 5G).

These data show that CD8⁺ T cell clones expressing heterologous high-affinity 5T4 TCRs recognize, selectively produce cytokines in response to, and kill peptide-pulsed T2 target cells.

### Example 4

### Specificity of 5T4-TCRs

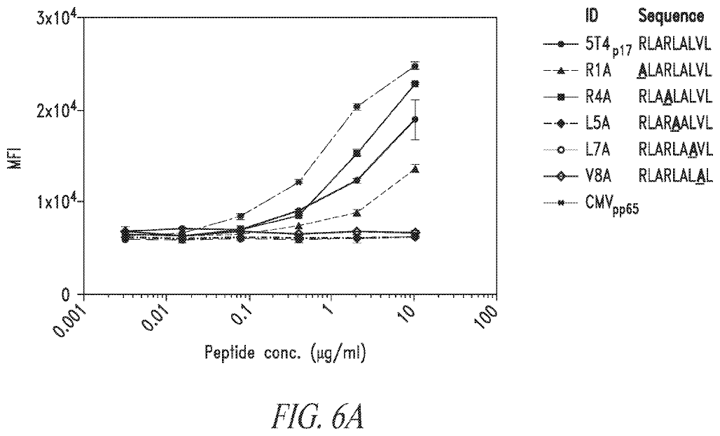

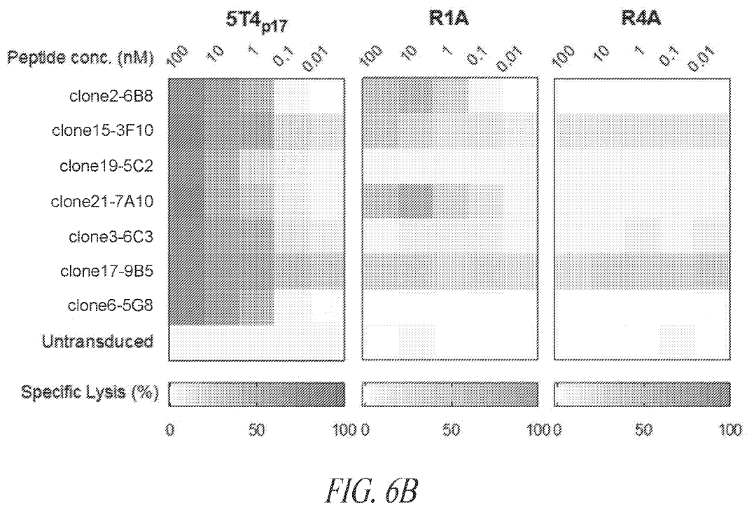

To further interrogate TCR-peptide:HLA binding specificity for 5T4ₚ₁₇-specific TCRs, five variant 5T4ₚ₁₇ peptides (“R1A,” “R4A,” “L5A,” “L7A,” “V8A”; FIG. 6A; SEQ ID NOs:168-172, respectively) were synthesized that contained individually substituted (non-alanine) residues each to the nonpolar, aliphatic alanine residue, except for the two conserved anchor residues essential for HLA-A2 binding (RLARLALVL (SEQ ID NO:64); lysine at position 2 and position 9; see Gfeller and Bassani-Sternberg, Front. Immunol. 9:1716 (2018)). HLA binding affinities of the five alanine-substituted peptides, as well as of the original 5T4ₚ₁₇, were measured by cell surface HLA-A2 stabilization on T2 cells and flow cytometry (FIG. 6A). The 5T4ₚ₁₇ (SEQ ID NO:64), R1A (SEQ ID NO:168), and R4A (SEQ ID NO:169) peptides stabilized HLA-A2 on the surface of T2 cells with a measurable increase of HLA-A2 staining. The alanine-substituted peptides L5A (SEQ ID NO:170), L7A (SEQ ID NO:171), and V8A (SEQ ID NO:172) showed no capacity to bind to HLA-A2, suggesting these side chains may contribute to the peptide-MHC binding interaction.

A cytotoxicity assay was performed with the seven 5T4ₚ₁₇-specific TCR expressing T cell lines and T2 targets pulsed with the 5T4ₚ₁₇, R1A, or R4A peptides. Despite stabilization of cell surface HLA-A2 by the R4A peptide comparable to 5T4ₚ₁₇, switching the positively charged arginine at position 4 to alanine disrupts completely T cell recognition for this sequence by all 7 of the 5T4ₚ₁₇-specific TCRs (FIG. 6B). The R1A peptide also stabilized cell surface HLA-A2 on T2 cells, and the arginine to alanine change at position 1 differentially preserved 5T4ₚ₁₇-specific TCR recognition. Three of the seven 5T4ₚ₁₇ specific TCRs (HD_A-2, HD_A-15, and HD_B-21) killed T2 targets pulsed with R1A at high peptide concentration (FIG. 6B).

### Example 5

### 5T4-TCR-Transduced Effector T Cells Lyse 5T4⁺:HLA-A2⁺ Tumor Targets

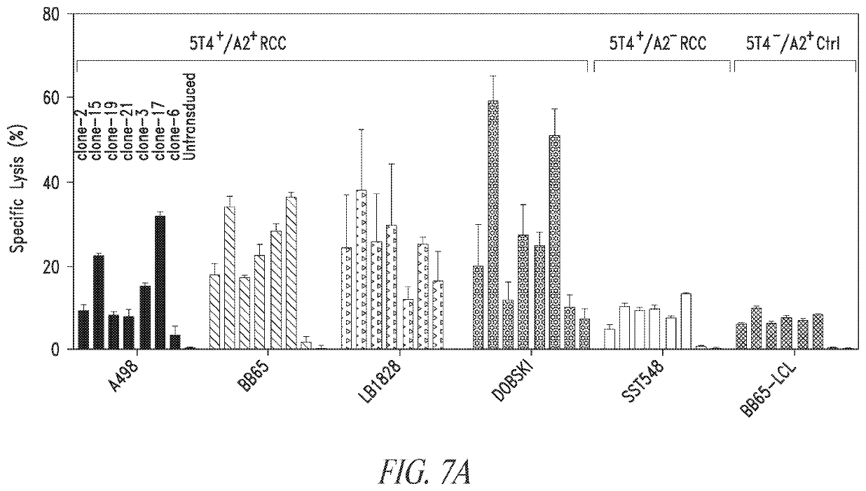

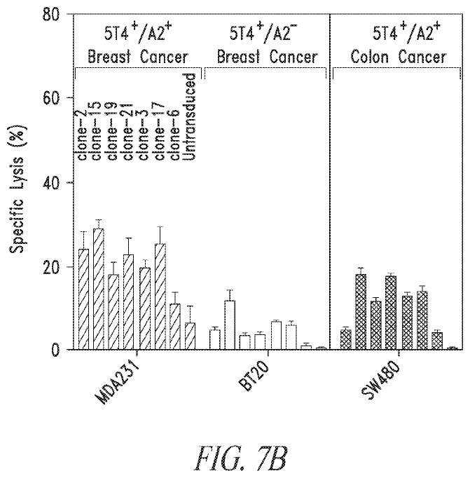

Cytotoxicity of 5T4ₚ₁₇-specific TCR-expressing T cells against 5T4⁺:HLA-A2⁺ RCC (A498, BB65, LB1828, DOBSKI), breast cancer (MDA231), and colon cancer (SW480) cell lines was tested versus HLA-mismatched (SST548, BT20) or 5T4-negative target cells (BB65-LCL). T cells expressing one of the seven 5T4ₚ₁₇-specific TCRs demonstrated specific lysis above background levels with control targets for at least one 5T4⁺:HLA-A2⁺ tumor line (FIGS. 7A and 7B). Among the seven 5T4ₚ₁₇-specific TCRs, T cells expressing clone15-3F10 and clone17-9B5 consistently had the highest lytic potency against all 5T4⁺:HLA-A2⁺ targets tested including RCC, breast cancer and colon cancer cell lines.

### Example 6

### 5T4-TCR-Transduced Effector T Cells Detect TAP1/2-Independent Processing of 5T4 Antigen in T2 Cells

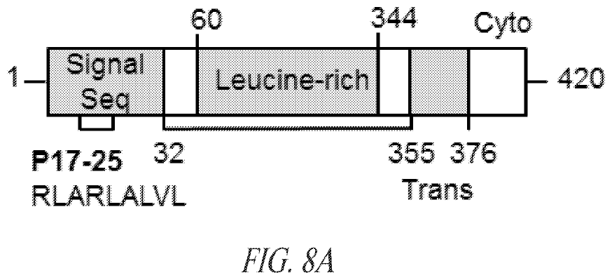

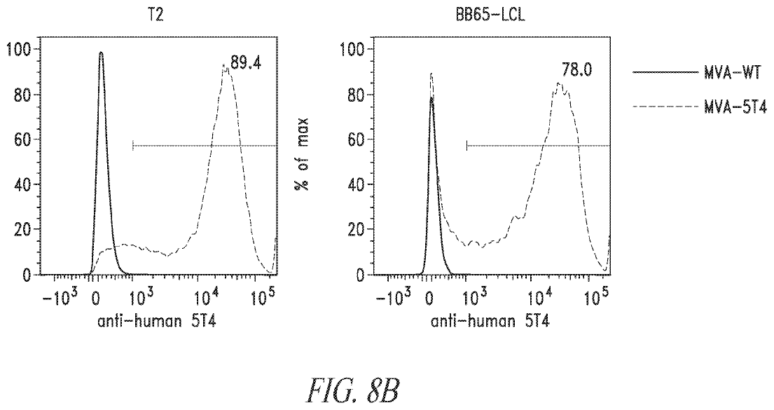

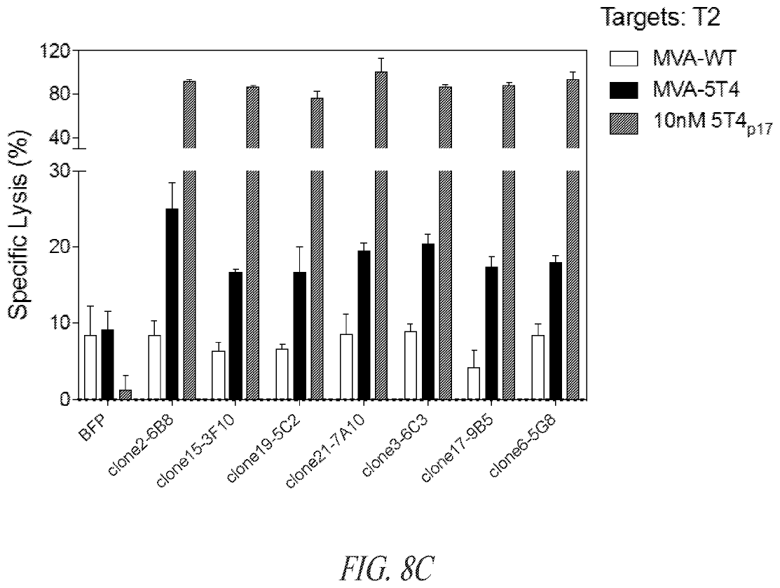

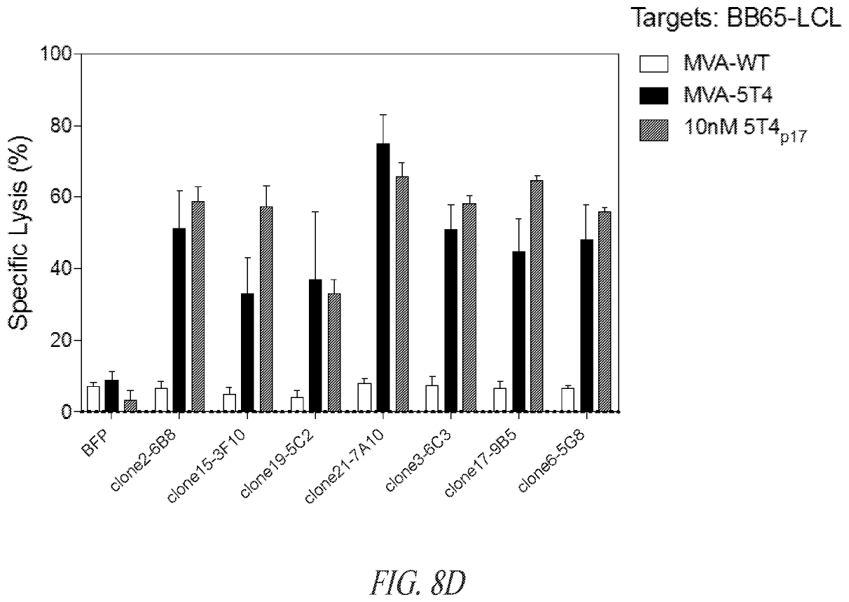

The amino acid sequence of 5T4p17 is located within the signal sequence domain of 5T4 (schematic shown in FIG. 8A). Some T cell epitopes encoded within protein leader domains have been shown to load MHC class I via a TAP-independent processing pathway as an alternative to classic antigen presentation pathway mediated by proteasome degradation and TAP transport processing steps (Blum et al., Annu. Rev. Immunol. 31:443-73 (2013)). TAP-independent processing may be advantageous for tumor-specific T cell immunity by facilitating T cell recognition of tumors with low expression of antigen-processing-associated genes and MHC class I proteins (Seliger et al., Oncotarget 7(41):67360-72 (2016)); Leone et al., J. Natl. Cancer Inst. 105(16):1172-87 (2013)). To assess TAP1/2-dependence for processing of the 5T4p17 epitope, 5T4p17-specific TCR-expressing T cells were tested for recognition of the antigen-processing mutant T2 cell line (HLA-A2⁺) expressing 5T4 versus a wild-type LCL target cell (BB65-LCL, HLA-A2+). T2 has bi-allelic deletions of chromosome 6 spanning the TAP1/2 genes (Salter et al., Immunogenetics 21β):235-46 (1985)). T2 cells and BB65-LCL were infected with a recombinant vaccinia virus encoding the full-length 5T4 gene (MVA-5T4) or wild-type virus (MVA-WT). Efficient target cell infection and high-level cell surface 5T4 expression was confirmed by immunostaining infected cells for 5T4 (FIG. 8B). 5T4-expressing T2 cells and BB65-LCL cells were then co-cultured with 5T4p17-specific TCR transduced CD8+ T cells. 5T4p17-specific T cells consistently recognized and lysed MVA-5T4 infected T2 cells above the background killing of MV-WT infected T2 targets (FIG. 8C), consistent with TAP1/2-independent processing and presentation of the 5T4p17 epitope in T2 cells. The specific lysis for 5T4-expressing T2 targets was in all cases less than for 5T4-expressing BB65-LCL (FIG. 8D). These data suggest that in the antigen-processing competent target cells, 5T4p17-processing and MHC class I presentation may result from both the classical processing pathway as well as by a TAP-independent mechanism. Furthermore, the robust specific lysis of 5T4ₚ₁₇-specific TCR transduced CD8⁺ T cells for MVA-5T4 infected LCL targets also suggests that subjects treated with 5T4ₚ₁₇-specific TCR engineered effectors T cells may be subsequently vaccinated with MVA-5T4 to re-stimulate the transduced effector T cells against the 5T4 target in vivo.

### Example 7

### Activity of Recombinant CD8⁺5 T4-TCR T Cells in a Xenograft Model

In vivo activity of the 5T4 TCR T cells is tested in a xenograft model of renal cell carcinoma. Briefly, immune-deficient NOD/SCID/IL2-Ry^{-/-} (“NSG”) mice are administered low-dose radiation (275 cGy) and inoculated in contralateral flanks with 1×10⁵ wild-type A498 RCC cells mixed with 1×10⁶ of CD8+ T cells transduced to express 5T4₁₇₋₂₅ specific TCRs or with a control clone KSN-7A7 specific for an antigen target not expressed on A498 (HLA-A3 antigen). Tumor cells and CD8⁺ T cells are mixed together in culture media, kept on ice, and 100 μl of the cell suspension is injected within 1 hour into mice. Alternatively, A498 tumor cells expressing firefly luciferase (see, e.g., Tykodi et al., J. Immunother. 35(7):523 (2012)) and CD8⁺ T cells are mixed together in culture media plus Matrigel (BD Biosciences) at a 1:1 (v/v) ratio in a final volume of 50 μl and implanted in the sub kidney capsule space of NSG mice. All mice receive 20,000 IU of IL-2 by intraperitoneal (i.p.) injection daily for 5 days following cell transfers.

Bioluminescent imaging is performed weekly for the A498-L tumors following i.p. injection of 150 mg/kg D-luciferin (Caliper Life Sciences, Hopkinton, Mass.) with an IVIS Spectrum imager and Living Image software (Caliper Life Sciences), and tumor progression is monitored. Mice are subsequently sacrificed and tumor sections are harvested for IHC staining (5T4 and HLA-A3).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 62/593,463, filed Dec. 1, 2017, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1.-165. (canceled)

166. A modified immune cell comprising a heterologous polynucleotide that encodes a binding protein, wherein the encoded binding protein comprises:
(a) a T cell receptor (TCR) α-chain variable (V_{α}) domain comprising a complementarity determining region (CDR) 1α having a net charge of about 0 to about −2.0, a CDR2α having a net charge of about 0 to about −1.0, and a CDR3α having a net charge of about −0.01 to about −2.2; and
(b) a TCR V_{β} domain comprising a CDR1β having a net charge of about −1.0 to about 1.5, a CDR2β having a net charge of about 0 to about −1.0, and a CDR3β having a net charge of about −0.01 to about −2.2,
wherein the encoded binding protein is capable of specifically binding to a 5T4 peptide:HLA-A*0201 complex, wherein the 5T4 peptide comprises or consists of (i) the amino acid sequence RLARLALVL (SEQ ID NO:64) or (ii) the amino acid sequence ALARLALVL (SEQ ID NO:168).

167. The modified immune cell of claim 166, wherein:
(a) the CDR1α, the CDR2α, the CDR3α, the CDR1β, the CDR2β, and the CDR3β each have a net charge that is about the same as the net charge of a corresponding CDR of Clone15-3F10, Clone17-9B5, Clone2-6B8, Clone6-5G8, Clone3-6C3, Clone19-5C2, or Clone21-7A10, as set forth in Table 1 and Table 2; or
(b) the CDR1α, the CDR2α, the CDR3α, the CDR1β, the CDR2β, and the CDR3β each have a net charge of a corresponding CDR of Clone15-3F10, Clone17-9B5, Clone2-6B8, Clone6-5G8, Clone3-6C3, Clone19-5C2, or Clone21-7A10, as set forth in Table 1 and Table 2.

168. The modified immune cell of claim 166, wherein the CDR1α has a net charge of about −1.0 or about −2.0, the CDR2α has a net charge of about 0, the CDR3α has a net charge of about −1.0, the CDR1β has a net charge of about −1.0, the CDR2β has a net charge of about −1.0, and the CDR3β has a net charge of about −1.0.

169. The modified immune cell of claim 166, wherein the encoded binding protein comprises the CDR3α amino acid sequence shown in any one of SEQ ID NOS.:52, 53, 49-51, 54, or 55, and the CDR3β amino acid sequence shown in any one of SEQ ID NOS.:59, 60, 56-58, 61, or 62.

170. The modified immune cell of claim 166, wherein:
(i) the encoded binding protein is capable of specifically binding to the RLARLALVL (SEQ ID NO:64):HLA-A*0201 complex with a K_{d} less than or equal to about 10⁻⁸M;
(ii) the encoded binding protein is capable of specifically binding to the 5T4 peptide:HLA-A*0201 complex on a cell surface independent of CD8 or in the absence of CD8; or
(iii) both of (i) and (ii).

171. The modified immune cell of claim 166, wherein the encoded binding protein comprises a pre-protein V_{α} domain that is at least about 90% identical to the amino acid sequence shown in any one of SEQ ID NOS.:4, 5, 1-3, 6, or 7, and comprises a pre-protein V_{β} domain that is at least about 90% identical to the amino acid sequence shown in any one of SEQ ID NOS.:11, 12, 8-10, 13, or 14,
provided that (a) at least three or four of the CDRs have no change in sequence, wherein the CDRs that do have sequence changes have only up to two to four amino acid substitutions, insertions, deletions, or a combination thereof, and (b) the encoded binding protein is capable of specifically binding to the 5T4 peptide:HLA complex on a cell surface.

172. The modified immune cell claim 166, wherein the encoded binding protein comprises (a) a pre-protein V_{α} domain that comprises or consists of the amino acid sequence shown in any one of SEQ ID NOS.:4, 5, 1-3, 6, or 7 and (b) a pre-protein V_{β} domain that comprises or consists of the amino acid sequence shown in any one of SEQ ID NOS.:11, 12, 8-10, 13, or 14.

173. The modified immune cell of claim 166, wherein the encoded binding protein further comprises an α-chain constant (C_{α}) domain having at least 90% sequence identity to the amino acid sequence shown in SEQ ID NO.:15, and a β-chain constant (C_{β}) domain having at least 90% sequence identity to the amino acid sequence shown in SEQ ID NO.:16 or 17.

174. The modified immune cell of claim 173, wherein the encoded binding protein comprises:
(i) a pre-protein V_{α} domain comprising or consisting of the amino acid sequence set forth in SEQ ID NO.:4, a pre-protein V_{β} domain comprising or consisting of the amino acid sequence set forth in SEQ ID NO.:11, a C_{α} domain comprising or consisting of the amino acid sequence set forth in SEQ ID NO.:15, and a C_{β} domain comprising or consisting of the amino acid sequence set forth in SEQ ID NO.:16 or SEQ ID NO.:17;
(ii) a pre-protein V_{α} domain comprising or consisting of the amino acid sequence set forth in SEQ ID NO.:5, a pre-protein V_{β} domain comprising or consisting of the amino acid sequence set forth in SEQ ID NO.:12, a C_{α} domain comprising or consisting of the amino acid sequence set forth in SEQ ID NO.:15, and a C_{β} domain comprising or consisting of the amino acid sequence set forth in SEQ ID NO.:16 or SEQ ID NO.:17;
(iii) a pre-protein V_{α} domain comprising or consisting of the amino acid sequence set forth in SEQ ID NO.:1, a pre-protein V_{β} domain comprising or consisting of the amino acid sequence set forth in SEQ ID NO.:8, a C_{α} domain comprising or consisting of the amino acid sequence set forth in SEQ ID NO.:15, and a C_{β} domain comprising or consisting of the amino acid sequence set forth in SEQ ID NO.:16 or SEQ ID NO.:17;
(iv) a pre-protein V_{α} domain comprising or consisting of the amino acid sequence set forth in SEQ ID NO.:2, a pre-protein V_{β} domain comprising or consisting of the amino acid sequence set forth in SEQ ID NO.:9, a C_{α} domain comprising or consisting of the amino acid sequence set forth in SEQ ID NO.:15, and a C_{β} domain comprising or consisting of the amino acid sequence set forth in SEQ ID NO:16 or SEQ ID NO.:17;
(v) a pre-protein V_{α} domain comprising or consisting of the amino acid sequence set forth in SEQ ID NO.:3, a pre-protein V_{β} domain comprising or consisting of the amino acid sequence set forth in SEQ ID NO.:10, a C_{α} domain comprising or consisting of the amino acid sequence set forth in SEQ ID NO.:15, and a C_{β} domain comprising or consisting of the amino acid sequence set forth in SEQ ID NO.:16 or SEQ ID NO.:17;
(vi) a pre-protein V_{α} domain comprising or consisting of the amino acid sequence set forth in SEQ ID NO.:6, a pre-protein V_{β} domain comprising or consisting of the amino acid sequence set forth in SEQ ID NO.:13, a C_{α} domain comprising or consisting of the amino acid sequence set forth in SEQ ID NO.:15, and a C_{β} domain comprising or consisting of the amino acid sequence set forth in SEQ ID NO.:16 or SEQ ID NO.:17; or
(vii) a pre-protein V_{α} domain comprising or consisting of the amino acid sequence set forth in SEQ ID NO.:7, a pre-protein V_{β} domain comprising or consisting of the amino acid sequence set forth in SEQ ID NO.:14, a C_{α} domain comprising or consisting of the amino acid sequence set forth in SEQ ID NO.:15, and a C_{β} domain comprising or consisting of the amino acid sequence set forth in SEQ ID NO.:16 or SEQ ID NO.:17.

175. The modified immune cell of claim 166, wherein the encoded binding protein comprises a T cell receptor (TCR), an antigen-binding fragment of a TCR, or is a chimeric antigen receptor (CAR).

176. The modified immune cell claim 166, wherein the modified immune cell comprises a chromosomal gene knockout of one or more of: a T Cell Receptor gene; a PD-1 gene; a LAG3 gene; a CTLA4 gene; a TIM3 gene; a HLA gene; or any combination thereof.

177. The modified immune cell of claim 166, wherein the immune cell comprises a T cell, a NK cell, or a NK-T cell.

178. The modified immune cell of claim 177, wherein the modified immune cell is capable of:
(I) specifically killing 50% or more of HLA-A2-expressing target cells in vitro, wherein the HLA-A2-expressing target cells are pulsed with a peptide or polypeptide comprising the amino acid sequence RLARLALVL (SEQ ID NO:64) at a concentration of about 1 nM to at least about 10 nM;
(ii) producing a cytokine when contacted in vitro with HLA-A2-expressing target cells pulsed with a peptide or polypeptide comprising or consisting of the amino acid sequence RLARLALVL (SEQ ID NO:64) at a concentration of about 1 nM to at least about 10 nM, wherein the cytokine comprises IFN-γ, TNF-α, or both;
(iii) specifically killing a cancer cell in vitro, wherein the cancer cell expresses:
(a) an HLA-A2 molecule; and
(b) a polypeptide comprising or consisting of the amino acid sequence RLARLALVL (SEQ ID NO:64); or
(iv) any combination of (i)-(iii).

179. The modified immune cell of claim 166, wherein the heterologous polynucleotide comprises a polynucleotide having at least 70%, 75%, 76%, 77%, 78%, 79%, 80%, 85%, 90%, 91%, 92%, 93%, 94%, 95%, 96%, 97%, 98%, 99%, or 100% sequence identity with the polynucleotide sequence of any one of SEQ ID NOS.:32-48, 65-70, and 112-146.

180. A fusion protein, comprising:
(a) an extracellular component that includes a binding domain, wherein the binding domain comprises:
(i) a T cell receptor (TCR) a chain variable (V_{α}) domain comprising the CDR3 amino acid sequence shown in any one of SEQ ID NOS:52, 53, 49-51, 54, or 55; and
(ii) a TCR V_{β} domain comprising the CDR3 amino acid sequence shown in any one of SEQ ID NOS:59, 60, 56-58, 61, or 62;
(b) an intracellular component comprising an effector domain or a functional portion thereof; and
(c) a transmembrane domain connecting the extracellular and intracellular components,
wherein the fusion protein is capable of specifically binding to a 5T4 peptide:HLA-A*0201 complex, wherein the 5T4 peptide comprises or consists of (i) the amino acid sequence RLARLALVL (SEQ ID NO:64) or (ii) the amino acid sequence ALARLALVL (SEQ ID NO:168).

181. A composition comprising a modified immune cell of claim 166 and a pharmaceutically acceptable carrier, diluent, or excipient.

182. An isolated polynucleotide encoding a binding protein, the polynucleotide comprising a polynucleotide encoding a T cell receptor (TCR) α-chain variable (V_{α}) domain comprising a CDR3 amino acid sequence of any one of SEQ ID NOS.:52, 53, 49-51, 54, or 55, wherein the polynucleotide encoding the V_{α} domain is at least 80% identical to the polynucleotide sequence of any one of SEQ ID NOS.: 35, 36, 32-34, 37, and 38; and a polynucleotide encoding a TCR β-chain variable (V_{β}) domain comprising a CDR3 amino acid sequence of any one of SEQ ID NOS.:59, 60, 56-58, 61, or 62, wherein the polynucleotide encoding the V_{β} domain is at least 80% identical to the polynucleotide sequence of any one of SEQ ID NOS.:42, 43, 39-41, 44, and 45,
wherein the encoded binding protein is capable of specifically binding to a RLARLALVL (SEQ ID NO:64):HLA-A*0201 complex.

183. An expression vector comprising an isolated polynucleotide according to claim 182 operably linked to an expression control sequence.

184. A method for treating a hyperproliferative disorder associated with 5T4 expression, the method comprising administering to human subject in need thereof a modified immune cell according to any one of claim 166.

185. An adoptive immunotherapy method for treating a condition characterized by 5T4 expression in cells of a subject having a hyperproliferative disorder, comprising administering to the subject an effective amount of a modified immune cell according to claim 166.
